SEQUENCE LISTING (117 sequences)

SEQ ID NO: 1 - protein, 242 aa, Artificial Sequence
  source: /note=“Description of Artificial Sequence Synthetic polypeptide”
  source: /note=“anti-CD19 ScFv”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 245 aa, Artificial Sequence
  source: /note=“Description of Artificial Sequence Synthetic polypeptide”
  source: /note=“FMC63”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 245 aa, Artificial Sequence
  source: /note=“Description of Artificial Sequence Synthetic polypeptide”
  source: /note=“RITUXIMAB”
SQIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKRSGGGGSGGGGSGGGGSGSQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYWGQGTTLTVSS

SEQ ID NO: 4 - protein, 271 aa, Artificial Sequence
  source: /note=“Description of Artificial Sequence Synthetic polypeptide”
  source: /note=“anti-CD20 ScFv”
DIQLTQSPAILSASPGEKVTMTCRASSSLSFMHWYQQKPGSSPKPWIYATSNLASGVPARFSGSGSGTSYSLTISTVEAEDAASYFCHQWSSNPLTFGAGTKLEISSGGGGSGGGGSGDVMGVDSGGGLVQPGGSRKLSCAAPGFTFSSFGMHWVRQAPEKGLEWVAYISSPSSTLHYADRVKGRFTISRDNPKNTLFLQMKLPSLCYGLLGPRDHVHRLLKHHHHHHLEYPYDVPDYATTDAAHPGRSVVPALLPLLAGTLLLLETATAP

SEQ ID NO: 5 - protein, 249 aa, Artificial Sequence
  source: /note=“Description of Artificial Sequence Synthetic polypeptide”
  source: /note=“anti-CD20 ScFv”
SQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSKISGGGGSGGGGSGGGGSGGSSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELK

SEQ ID NO: 6 - protein, 245 aa, Artificial Sequence; identical to SEQ ID NO: 3
  source: /note=“Description of Artificial Sequence Synthetic polypeptide”
  source: /note=“anti-CD20 ScFv”

SEQ ID NO: 7 - protein, 441 aa, Artificial Sequence
  source: /note=“Description of Artificial Sequence Synthetic polypeptide”
  source: /note=“anti-desmo 3 (domain 1-4)”
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFND

SEQ ID NO: 8 - protein, 16 aa, Artificial Sequence
  source: /note=“Description of Artificial Sequence Synthetic peptide”
  source: /note=“Linker”
AGSSSSGGSTTGGSTT

SEQ ID NO: 9 - protein, 16 aa, Artificial Sequence
  source: /note=“Description of Artificial Sequence Synthetic peptide”
  source: /note=“Linker”
GTTAASGSSGGSSSGA

SEQ ID NO: 10 - protein, 16 aa, Artificial Sequence
  source: /note=“Description of Artificial Sequence Synthetic peptide”
  source: /note=“Linker”
SSATATAGTGSSTGST

SEQ ID NO: 11 - protein, 16 aa, Artificial Sequence
  source: /note=“Description of Artificial Sequence Synthetic peptide”
  source: /note=“Linker”
TSGSTGTAASSTSTST

SEQ ID NO: 12 - DNA, 30 bp, Artificial Sequence
  source: /note=“Description of Artificial Sequence Synthetic oligonucleotide”
  source: /note=“Linker”
ggtggcggaggttctggaggtggaggttcc

SEQ ID NO: 13 - protein, 7 aa, Homo sapiens
  source: /note=“KIR2DS2 hinge domain”
KIRRDSS

SEQ ID NO: 14 - protein, 45 aa, Homo sapiens
  source: /note=“CD8 hinge domain”
TTTPAPRPPTPAPTIASQPLSLRPEACRPAAGGAVHTRGLDFACD

SEQ ID NO: 15 - DNA, 135 bp, Homo sapiens
  source: /note=“CD8 hinge domain”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 16 - protein, 230 aa, Homo sapiens
  source: /note=“IgG4 hinge domain”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 17 - DNA, 690 bp, Homo sapiens
  source: /note=“IgG4 hinge domain”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 18 - protein, 282 aa, Homo sapiens
  source: /note=“IgD hinge domain”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 19 - DNA, 847 bp, Homo sapiens
  source: /note=“IgD hinge domain”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 20 - protein, 39 aa, Homo sapiens
  source: /note=“CD28 hinge domain”
IEVMYPPPYLDNEKSNGTIIHVKGKHLCPSPLFPGPSKP

SEQ ID NO: 21 - DNA, 117 bp, Homo sapiens
  source: /note=“CD28 hinge domain”
attgaagttatgtatcctcctccttacctagacaatgagaagagcaatggaaccattatccatgtgaaagggaaacacctttgtccaagtcccctatttcccggaccttctaagccc

SEQ ID NO: 22 - protein, 30 aa, Homo sapiens
  source: /note=“TNFR2 TM domain”
FALPVGLIVGVTALGLLIIGVVNCVIMTQV

SEQ ID NO: 23 - DNA, 90 bp, Homo sapiens
  source: /note=“TNFR2 TM domain”
ttcgctcttccagttggactgattgtgggtgtgacagccttgggtctactaataataggagtggtgaactgtgtcatcatgacccaggtg

SEQ ID NO: 24 - protein, 24 aa, Homo sapiens
  source: /note=“CD8 TM”
IYIWAPLAGTCGVLLLSLVITLYC

SEQ ID NO: 25 - DNA, 72 bp, Homo sapiens
  source: /note=“CD8 TM”
atctacatctgggcgcccttggccgggacttgtggggtccttctcctgtcactggttatcaccctttactgc

SEQ ID NO: 26 - protein, 27 aa, Homo sapiens
  source: /note=“CD28 TM”
FWVLVVVGGVLACYSLLVTVAFIIFWV

SEQ ID NO: 27 - DNA, 81 bp, Homo sapiens
  source: /note=“CD28 TM”
ttttgggtgctggtggtggttggtggagtcctggcttgctatagcttgctagtaacagtggcctttattattttctgggtg

SEQ ID NO: 28 - protein, 112 aa, Homo sapiens
  source: /note=“CD3 zeta”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 112 aa, Homo sapiens
  source: /note=“CD3 zeta”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 113 aa, Homo sapiens
  source: /note=“CD3 zeta”
RVKFSRSADAPAYKQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 113 aa, Homo sapiens
  source: /note=“CD3 zeta”
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - DNA, 336 bp, Homo sapiens
  source: /note=“CD3 zeta”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - DNA, 336 bp, Homo sapiens
  source: /note=“CD3 zeta”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 174 aa, Homo sapiens
  source: /note=“TNFR2 intracellular”
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 35 - DNA, 522 bp, Homo sapiens
  source: /note=“TNFR2 intracellular”
aaaaagaagcccttgtgcctgcagagagaagccaaggtgcctcacttgcctgcagataaggccaggggtacacagggtcccgagcagcagcacctcctgatcacagcgccgagctccagcagcagctccctggagagctcggccagtgcgctagacagaagggcccccactcggaaccagccacaggcaccaggcgtggaggcgagtggggctggggaggcgagggcaagcaccgggagctcagattcttcccctggtggtcatggcacccaggtcaatgtcacctgtatcgtgaacgtctgtagcagctctgaccacagctcacagtgctcctcccaagcctcgtccacaatgggagacacagattccagcccctcggagtccccgaaggacgagcaagtacccttctccaaggaggaatgtgcctttcggtcacagctggagacgccagagaccctgctggggagcaccgaagagaagcccctgccccttggagtgcctgatgctgggatgaagcccagt

SEQ ID NO: 36 - protein, 42 aa, Homo sapiens
  source: /note=“4-1BB intracellular”
KRGRKKLLYIFKQPFMRPVQTTQEEDGCSCRFPEEEEGGCEL

SEQ ID NO: 37 - DNA, 126 bp, Homo sapiens
  source: /note=“4-1BB intracellular”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 38 - protein, 48 aa, Homo sapiens
  source: /note=“CD27 intracellular”
QRRKYRSNKGESPVEPAEPCRYSCPREEEGSTIPIQEDYRKPEPACSP

SEQ ID NO: 39 - DNA, 144 bp, Homo sapiens
  source: /note=“CD27 intracellular”
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 40 - protein, 41 aa, Homo sapiens
  source: /note=“CD28 intracellular”
RSKRSRLLHSDYMNMTPRRPGPTRKHYQPYAPPRDFAAYRS

SEQ ID NO: 41 - DNA, 123 bp, Homo sapiens
  source: /note=“CD28 intracellular”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 42 - protein, 21 aa, Artificial Sequence
  source: /note=“Description of Artificial Sequence Synthetic peptide”
  source: /note=“Leader sequence CD8”
MALPVTALLLPLALLLHAARP

SEQ ID NO: 43 - protein, 9 aa, Artificial Sequence
  source: /note=“Description of Artificial Sequence Synthetic peptide”
  source: /note=“tag”
NWSHPQFEK

SEQ ID NO: 44 - protein, 10 aa, Artificial Sequence
  source: /note=“Description of Artificial Sequence Synthetic peptide”
  source: /note=“tag”
SAWSHPQFEK

SEQ ID NO: 45 - protein, 22 aa, Artificial Sequence
  source: /note=“Description of Artificial Sequence Synthetic peptide”
  source: /note=“P2A tag”
GSGATNFSLLKQAGDVEENPGP

SEQ ID NO: 46 - protein, 239 aa, Artificial Sequence
  source: /note=“Description of Artificial Sequence Synthetic polypeptide”
  source: /note=“GFP tag”
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 47 - protein, 12 aa, Artificial Sequence
  source: /note=“Description of Artificial Sequence Synthetic peptide”
  source: /note=“Strepavidin tag”
SAWSHPQFEKSG

SEQ ID NO: 48 - protein, 365 aa, Artificial Sequence
  source: /note=“Description of Artificial Sequence Synthetic polypeptide”
  source: /note=“CD8 H-TNFR2TM-TNFR2-3z”
TTTPAPRPPTPAPTIASQPLSLRPEACRPAAGGAVHTRGLDFACDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 49 - protein, 347 aa, Artificial Sequence
  source: /note=“Description of Artificial Sequence Synthetic polypeptide”
  source: /note=“CD8 H-TNFR2 TM-TNFR2del18-3z”
TTTPAPRPPTPAPTIASQPLSLRPEACRPAAGGAVHTRGLDFACDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 50 - protein, 306 aa, Artificial Sequence
  source: /note=“Description of Artificial Sequence Synthetic polypeptide”
  source: /note=“CD8 H-TNFR2 TM-TNFR2del59-3z”
TTTPAPRPPTPAPTIASQPLSLRPEACRPAAGGAVHTRGLDFACDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 51 - protein, 261 aa, Artificial Sequence
  source: /note=“Description of Artificial Sequence Synthetic polypeptide”
  source: /note=“CD8 H-TNFR2 TM-TNFR2del104-3z”
TTTPAPRPPTPAPTIASQPLSLRPEACRPAAGGAVHTRGLDFACDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 52 - protein, 189 aa, Artificial Sequence
  source: /note=“Description of Artificial Sequence Synthetic polypeptide”
  source: /note=“CD8 H-TNFR2 TM-3z”
TTTPAPRPPTPAPTIASQPLSLRPEACRPAAGGAVHTRGLDFACDFALPVGLIVGVTALGLLIIGVVNCVIMTQVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 53 - protein, 361 aa, Artificial Sequence
  source: /note=“Description of Artificial Sequence Synthetic polypeptide”
  source: /note=“CD8 H-3z-TNFR2 TM”
TTTPAPRPPTPAPTIASQPLSLRPEACRPAAGGAVHTRGLDFACDIYIWAPLAGTCGVLLLSLVITLYCTRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSAS

SEQ ID NO: 54 - protein, 359 aa, Artificial Sequence
  source: /note=“Description of Artificial Sequence Synthetic polypeptide”
  source: /note=“CD8 H-CD8 TM- TNFR2-3z”
TTTPAPRPPTPAPTIASQPLSLRPEACRPAAGGAVHTRGLDFACDIYIWAPLAGTCGVLLLSLVITLYCKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 55 - protein, 363 aa, Artificial Sequence
  source: /note=“Description of Artificial Sequence Synthetic polypeptide”
  source: /note=“CD8 H-FUSED 1 TM- TNFR2-3z”
TTTPAPRPPTPAPTIASQPLSLRPEACRPAAGGAVHTRGLDFACDIYIWAPLAGTCGVLLLSLVITCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 56 - protein, 365 aa, Artificial Sequence
  source: /note=“Description of Artificial Sequence Synthetic polypeptide”
  source: /note=“CD8 H-FUSED 2TM- TNFR2-3z”
TTTPAPRPPTPAPTIASQPLSLRPEACRPAAGGAVHTRGLDFACDIYIWAPLAGTCGVLLLSLVITVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 57 - protein, 363 aa, Artificial Sequence
  source: /note=“Description of Artificial Sequence Synthetic polypeptide”
  source: /note=“CD8 H-FUSED 3 TM- TNFR2-3z”
TTTPAPRPPTPAPTIASQPLSLRPEACRPAAGGAVHTRGLDFACDIYIWAPLAGTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 58 - protein, 9 aa, Artificial Sequence
  source: /note=“Description of Artificial Sequence Synthetic peptide”
  source: /note=“LAGLIDADG motif”
LAGLIDADG

SEQ ID NO: 59 - protein, 21 aa, Artificial Sequence
  source: /note=“Description of Artificial Sequence Synthetic peptide”
  source: /note=“Fusion 1 - CD8 SEQ”
IYIWAPLAGTCGVLLLSLVIT

SEQ ID NO: 60 - protein, 21 aa, Artificial Sequence
  source: /note=“Description of Artificial Sequence Synthetic peptide”
  source: /note=“Fusion 2 - CD8 SEQ”
IYIWAPLAGTCGVLLLSLVIT

SEQ ID NO: 61 - protein, 9 aa, Artificial Sequence
  source: /note=“Description of Artificial Sequence Synthetic peptide”
  source: /note=“Fusion 3 - CD8 SEQ”
IYIWAPLAG

SEQ ID NO: 62 - protein, 7 aa, Artificial Sequence
  source: /note=“Description of Artificial Sequence Synthetic peptide”
  source: /note=“Fusion 1 - TNFR2 SEQ”
CVIMTQV

SEQ ID NO: 63 - protein, 9 aa, Artificial Sequence
  source: /note=“Description of Artificial Sequence Synthetic peptide”
  source: /note=“Fusion 2 - TNFR2 SEQ”
VNCVIMTQV

SEQ ID NO: 64 - protein, 19 aa, Artificial Sequence
  source: /note=“Description of Artificial Sequence Synthetic peptide”
  source: /note=“Fusion 3 - TNFR2 SEQ”
TALGLLIIGVVNCVIMTQV

SEQ ID NO: 65 - protein, 249 aa, Artificial Sequence
  source: /note=“Description of Artificial Sequence Synthetic polypeptide”
  source: /note=“scFv anti-IL-23R”
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLGGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 66 - protein, 249 aa, Artificial Sequence
  source: /note=“Description of Artificial Sequence Synthetic polypeptide”
  source: /note=“scFv anti-IL-23R”
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGGGTKLTVLGGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 67 - protein, 249 aa, Artificial Sequence
  source: /note=“Description of Artificial Sequence Synthetic polypeptide”
  source: /note=“scFv anti IL-23R”
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGTGTKVTVLGGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 68 - protein, 251 aa, Artificial Sequence
  source: /note=“Description of Artificial Sequence Synthetic polypeptide”
  source: /note=“anti-HLA-A2 scFv”
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSVDSSGGGGSGGGGSGGGGSTSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 69 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTTVTVSSVDSSGGGGSGGGGSGGGGSTSDVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHVPRTFGGGTKVEIK

SEQ ID NO: 70 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 71 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTTVTVSSVDSSGGGGSGGGGSGGGGSTSDIVMTQTPLSSPVTLGQPASISCRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 72 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTTVTVSSVDSSGGGGSGGGGSGGGGSTSDVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPRTFGGGTKVEIK

SEQ ID NO: 73 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTTVTVSSVDSSGGGGSGGGGSGGGGSTSDIVMTQTPLSSPVTLGQPASISFRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 74 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITADTSASTAYMLLSSLRSEDTAVYYCAREGTYYAMDYWGQGTTVTVSSVDSSGGGGSGGGGSGGGGSTSDVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHVPRTFGGGTKVEIK

SEQ ID NO: 75 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITADTSASTAYMLLSSLRSEDTAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 76 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGIWIYPGDGSTQYNEKFKGVTMTRDTSTSTVYMELSSLRSEDTAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 77 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGIWIYPGDGSTQYNEKFKGVTMTRDTSTSTVYMELSSLRSEDTAVYYCAREGTYYAMDYWGQGTTVTVSSVDSSGGGGSGGGGSGGGGSTSDIVMTQTPLSSPVTLGQPASISCRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 78 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGIWIYPGDGSTQYNEKFKGVTMTRDTSTSTVYMELSSLRSEDTAVYYCAREGTYYAMDYWGQGTTVTVSSVDSSGGGGSGGGGSGGGGSTSDVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPRTFGGGTKVEIK

SEQ ID NO: 79 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGIWIYPGDGSTQYNEKFKGVTMTRDTSTSTVYMELSSLRSEDTAVYYCAREGTYYAMDYWGQGTTVTVSSVDSSGGGGSGGGGSGGGGSTSDIVMTQTPLSSPVTLGQPASISFRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 80 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGWIYPGDGSTQYNEKFKGRVTMTRDTSISTAYMELSRLRSDDTAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 81 - protein, 251 aa, Artificial Sequence; identical to SEQ ID NO: 80
  source: /note=“Description of Artificial Sequence Synthetic polypeptide”
  source: /note=“anti-HLA-A2 scFv”

SEQ ID NO: 82 - protein, 251 aa, Artificial Sequence; identical to SEQ ID NO: 80
  source: /note=“Description of Artificial Sequence Synthetic polypeptide”
  source: /note=“anti-HLA-A2 scFv”

SEQ ID NO: 83 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQGLEWMGWIYPGDGSTQYNEKFKGRVTMTRDTSISTAYMELSRLRSDDTAVYYCAREGTYYAMDYWGQGTTVTVSSVDSSGGGGSGGGGSGGGGSTSDIVMTQTPLSSPVTLGQPASISFRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 84 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDMAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 85 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDMAVYYCAREGTYYAMDYWGQGTTVTVSSVDSSGGGGSGGGGSGGGGSTSDIVMTQTPLSSPVTLGQPASISCRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 86 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDMAVYYCAREGTYYAMDYWGQGTTVTVSSVDSSGGGGSGGGGSGGGGSTSDVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPRTFGGGTKVEIK

SEQ ID NO: 87 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTQYNEKFKGRVTITRDTSASTAYMELSSLRSEDMAVYYCAREGTYYAMDYWGQGTTVTVSSVDSSGGGGSGGGGSGGGGSTSDIVMTQTPLSSPVTLGQPASISFRSSQSIVHSNGNTYLEWYQQRPGQPPRLLIYKVSNRFSGVPDRFSGSGAGTDFTLKISRVEAEDVGVYYCFQGSHVPRTFGGGTKVEIK

SEQ ID NO: 88 - protein, 251 aa, Artificial Sequence
  source: /note=“Description of Artificial Sequence Synthetic polypeptide”
  source: /note=“anti-HLA-A2 scFv”
QVQLVQSGAEVKKPGASVKVSCKASGYTFTSYHIQWVRQAPGQRLEWMGWIYPGDGSTKYSQKFQGRVTITRDTSASTAYMELSSLRSEDTAVYYCAREGTYYAMDYWGQGTTVTVSSVDSSGGGGSGGGGSGGGGSTSDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 89 - protein, 251 aa, Artificial Sequence
  source: /note=“Description of Artificial Sequence Synthetic polypeptide”
  source: /note=“anti-HLA-A2 scFv”
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYDSRGSYYYMDVWGKGTTVTVSSGSASAPKLEEGEFSEARVQSVLTQPPSTSGTPGQRVTISCSGSSSNIGGNAVNWYQHFPGTAPTLLIYSNNQRPSGVPERFSGSKSGTSASLTVSGLQAEDEADYYCTAWDDSLRGYLFGTGTKVTVL

SEQ ID NO: 90 - protein, 249 aa, Artificial Sequence
  source: /note=“Description of Artificial Sequence Synthetic polypeptide”
  source: /note=“anti-HLA-A2 scFv”
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDREELLALFGGMDVWGQGTTVTVSSGSASAPKLEEGEFSEARVQPVLTQPSSVSVAPGQTARITCGGNNIGSKSVHWYQQKPGQAPVLVVYDDSDRPSGIPERFSGSNSGNTATLTISRVEARDEADYYCHVWDAKTNHQVFGGGTRLTVQ

SEQ ID NO: 91 - protein, 251 aa, Artificial Sequence
  source: /note=“Description of Artificial Sequence Synthetic polypeptide”
  source: /note=“anti-HLA-A2 scFv”
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPQSRWLQSGDAFDIWGQGTMVTVSSGSASAPKLEEGEFSEARVQPVLTQPRSVSGSPGQSVTISCTGTSSDVGGYNRVSWYQQTPGTAPKLMIYEVSNRPSGVSNRFSGSKSGNTASLTISGLQAEDEADYYCSSYTSSSTVVFGGGTKLTVL

SEQ ID NO: 92 - protein, 246 aa, Artificial Sequence
  source: /note=“Description of Artificial Sequence Synthetic polypeptide”
  source: /note=“anti-HLA-A2 scFv”
QVQLQQSGAEVKKPGASVKVSCKASGYTFTSYAMHWVRQAPGQRLEWMGRINAGNGNTKYSQKFQGRVTITRDTSASTAYMELSSLRSEDTAVYYCARDLTGTLLFDYWGQGTLVTVSSGSASAPKLEEGEFSEARVQSVLTQPPSASGTPGQRVTISCSGSSSNIGSNGVKWYQQLPGTAPKLVIYRDYQRPSGVPDRFSGSKSGTSASLAISGLQSEDEAKYYCAAWDDSLNVVFGGGTQLTVL

SEQ ID NO: 93 - protein, 251 aa, Artificial Sequence
  source: /note=“Description of Artificial Sequence Synthetic polypeptide”
  source: /note=“anti-HLA-A2 scFv”
QVQLVQSGAEVKKPGSSVKVSCKASGGTFSSYAISWVRQAPGQGLEWMGGIIPIFGTANYAQKFQGRATITADESTSTAYMELSSLRSEDTAVYYCARRAERWLHLSGAFDIWGQGTMVTVSSGSASAPKLEEGEFSEARVQPVLTQSSSASGTPGQRVAISCSGSSSNVGSNTVNWYQQSPGTAPKLLISSNHQRPSGVPDRFSGSKFGTSASLAISGLQSEDEADYYCGAWDDSLNGYVFGSGTKVTVL

SEQ ID NO: 94 - protein, 244 aa, Artificial Sequence
  source: /note=“Description of Artificial Sequence Synthetic polypeptide”
  source: /note=“anti-HLA-A2 scFv”
QVQLVQSGGGLVQPGGSLRLSCAASGFTFSSYAMSWVRQAPGKGLEWVSAISGSGGSTYYADSVKGRFTMSRDNAKNSLYLQMNSLRVEDSAVYYCATGHYGDYVWGQGALVTVSSGSASAPKLEEGEFSEARVQAGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 95 - protein, 246 aa, Artificial Sequence
  source: /note=“Description of Artificial Sequence Synthetic polypeptide”
  source: /note=“anti-HLA-A2 scFv”
QVQLQQSGAEVKKPGASVKVSCKASGYTFTSYAMHWVRQAPGQRLEWMGWINAGNGNTKYSQKFQGRVTITRDTSASTAYMELSSLRSEDTAVYYCARVSSGGAFDIWGQGTVVTVSSGSASAPKLEEGEFSEARVQSALTQPASVSGSPGQSITISCTGTGSDVGGYKYVSWYQHHPGKAPRLIIYDVNYWPSGVSHRFSGSKSGNTASLTISGLQSEDEADYYCSSYRTGDTWVFGGGTKLTVL

SEQ ID NO: 96 - protein, 248 aa, Artificial Sequence
  source: /note=“Description of Artificial Sequence Synthetic polypeptide”
  source: /note=“anti-HLA-A2 scFv”
QVQLVQSGAEVKKPGSSVKVSCKASGGTFSSYGFSWVRQAPGQGLEWMGEIIPMFGTANYAQKLQGRVTITAETSTSTVYMELSSLRSEDTATYYCARVPRSSSGYNYGMDVWGQGTTVTVSSGSASAPKLEEGEFSEARVDIQMTQSPSSLSASVGDRVTITCRASQGISNYLAWYQQKPGKVPKLLIYAASTLQSGVPSRFSGSGSGTDFTLTISSLQPEDVATYYCQKYNSAPRTFGQGTKVEIK

SEQ ID NO: 97 - protein, 250 aa, Artificial Sequence
  source: /note=“Description of Artificial Sequence Synthetic polypeptide”
  source: /note=“anti-HLA-A2 scFv”
QVQLQQSGPGLLKPSQTLSLTCAVSGDSVSTNSGAWSWIRQSPSRGLEWLGRTYYRSKWSTDYALSLQSRVTIKSDRSKNQFSLQLDSVTPEDTAIYYCARENWNSGGFDYWGQGTLVTVPSGSASAPKLEEGEFSEARVQPVLTQSSSASGSPGQSVTISCTGTSSDVGGYNYVSWYQQHPGKAPKLMIYEVSKRPSGVPDRFSGSKSGSTASLTVSGLQAEDEAEYYCSSYAGSNNYVFGTGTKVTVL

SEQ ID NO: 98 - protein, 249 aa, Artificial Sequence
  source: /note=“Description of Artificial Sequence Synthetic polypeptide”
  source: /note=“anti-HLA-A2 scFv”
EVQLVESGAEVKKPGSSVKVSCKASGGTFSSYAISWVRQAPGQGLEWMGGIIPIFGTANYAQKFQGRVTITADKSTSTAYMELSSLRSEDTAVYYCARAASRWEPGDAFDIWGQGTMVTVSSGSASAPKLEEGEFSEARVQPVLTQSSSVSVAPGKTARVTCGGDNIGGKSVHWYQQRAGQAPVLVISHDTDRPSGIPERFSGSKSGTSASLAISGLRSEDEADYYCAVWDASLGGSWLFGGGTKLTVL

SEQ ID NO: 99 - protein, 252 aa, Artificial Sequence
  source: /note=“Description of Artificial Sequence Synthetic polypeptide”
  source: /note=“anti-HLA-A2 scFv”
QVQLVQSGAEVKKPGASVKVSCKTSGYTFTSYDISWVRQAPGQGLEWMGWISAYNGNTNYAQKLQGRVTMTTDTSTSTAYMELRRLRSDDTAVYYCARGGRWLRSASSFDYWGQGTLVTVSSGSASAPKLEEGEFSEARVQAGLTQPPSVSGAPGQRVTISCTGSSSNIGAAYDVHWYQQLPGAAPKLLIFGDSNRPSGVPDRFSGSKSDTSASLAITGLQAEDEADYYCQSFDSSLSGSRVFGGGTKLTVL

SEQ ID NO: 100 - protein, 251 aa, Artificial Sequence
  source: /note=“Description of Artificial Sequence Synthetic polypeptide”
  source: /note=“anti-HLA-A2 scFv”
EVQLVESGGGLVKPGGSLRLSCAASGFTFSDHYMSWVRQAPGKGLEWVSYITSGGSSIYYADSVKGRFTISRDNAKNSLYLQMNSLRAEDTAVYYCARGLDSSAYQGRAFDIWGQGTMVTVSSGSASAPKLEEGEFSEARVLPVLTQPPSASGTPGQRVTISCSGSSSNIGSNPVHWYQQLPGTAPKLLVYRNNQRPSGVPDRFSGSKSGTSASLAISGLRSEDEADYYCAAWDVSLSGVVFGGGTKLTVL

SEQ ID NO: 101 - protein, 246 aa, Artificial Sequence
  source: /note=“Description of Artificial Sequence Synthetic polypeptide”
  source: /note=“anti-HLA-A2 scFv”
QVQLVQSGPEVKKPGASVKVSCKASGYTFTSYHIQWVRQAPGQGLEWIGWIYPGDGSTQYNEKFKGRVTITADKSTSTAYMELSSLTSEDTAVYYCAREGTYYAMDYWGQGTSVTVSSGGGGSGGGGSGGGGSDVVMTQTPLSLPVTLGEPASISCRSSQSIVHSNGNTYLEWYLQKPGQSPRLLIYKVSNRFSGVPDRFSGSGSGTDFTLKISRVEAEDLGVYYCFQGSHVPRTFGGGTKLEIKR

SEQ ID NO: 102 - protein, 246 aa, Artificial Sequence
  source: /note=“Description of Artificial Sequence Synthetic polypeptide”
  source: /note=“anti-HLA-A2 scFv”
QVQLVQSGPEVKKPGASVKVSCKASGYTFTSYHIQWVRQAPGQGLEWIGWIYPGDGSTQYNEKFKGRVTITADKSTSTAYMELSSLTSEDTAVYYCAREGTYYAMDYWGQGTSVTVSSGGGGSGGGGSGGGGSDVVMTQSPSSLSVTLGDRVSISCRSSQSIVHSNGNTYLEWYQQKPGQSPRLLIYKVSNRFSGVPDRFSGSGSGTDFTLTISRVEPEDLGVYYCFQGSHVPRTFGGGTKLEIKR

SEQ ID NO: 103 - protein, 246 aa, Artificial Sequence
  source: /note=“Description of Artificial Sequence Synthetic polypeptide”
  source: /note=“anti-HLA-A2 scFv”
QVQLVQSGPEVKKPGASVKVSCKASGYTFTSYHIQWVRQAPGQGLEWIGWIYPGDGSTQYNEKFKGRVTITADKSTSTAYMELSSLTSEDTAVYYCAREGTYYAMDYWGQGTSVTVSSGGGGSGGGGSGGGGSDIVMTQSPATLSVSPGERATISCRSSQSIVHSNGNTYLEWYQQKPGQAPRLLIYKVSNRFSGIPDRFSGSGSGTDFTLTISRLEPEDFAVYYCFQGSHVPRTFGGGTKLEIKR

SEQ ID NO: 104 - protein, 247 aa, Artificial Sequence
  source: /note=“Description of Artificial Sequence Synthetic polypeptide”
  source: /note=“anti-HLA-A2 scFv”
SQVQLVQSGPEVKKPGASVKVSCKASGYTFTSYHIQWVRQAPGQGLEWIGWIYPGDGSTQYNEKFKGRVTITADKSTSTAYMELSSLTSEDTAVYYCAREGTYYAMDYWGQGTSVTVSSGGGGSGGGGSGGGGSDVVMTQSPSSLSVTLGDRVSISCRSSQSIVHSNGNTYLEWYQQKPGQSPRLLIYKVSNRFSGVPDRFSGSGSGTDFTLTISRVEPEDLGVYYCFQGSHVPRTFGGGTKLEIKR

SEQ ID NO: 105 - protein, 247 aa, Artificial Sequence; identical to SEQ ID NO: 104
  source: /note=“Description of Artificial Sequence Synthetic polypeptide”
  source: /note=“anti-HLA-A2 scFv”

SEQ ID NO: 106 - protein, 247 aa, Artificial Sequence
  source: /note=“Description of Artificial Sequence Synthetic polypeptide”
  source: /note=“anti-HLA-A2 scFv”
SQVQLVQSGPEVKKPGASVKVSCKASGYTFTSYHIQWVRQAPGQGLEWIGWIYPGDGSTQYNEKFKGRVTITADKSTSTAYMELSSLTSEDTAVYYCAREGTYYAMDYWGQGTSVTVSSGGGGSGGGGSGGGGSDIVMTQSPATLSVSPGERATISCRSSQSIVHSNGNTYLEWYQQKPGQAPRLLIYKVSNRFSGIPDRFSGSGSGTDFTLTISRLEPEDFAVYYCFQGSHVPRTFGGGTKLEIKR

SEQ ID NO: 107 - protein, 245 aa, Artificial Sequence
  source: /note=“Description of Artificial Sequence Synthetic polypeptide”
  source: /note=“anti-HLA-A2 scFv”
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIKR

SEQ ID NO: 108 - DNA, 2853 bp, Artificial Sequence
  source: /note=“Description of Artificial Sequence Synthetic polynucleotide”
  source: /note=“CD20-CAR (TNFR1TM-TNFR1-CD3z)-P2A-GFP”
atggctctgcctgtgacagctctgctgctgcctctggctctgcttctgcatgccgccagaccttctcaggtgcagctggttcagtctggcgccgagcttgtgaaacctggcgcctctgtgaagatgagctgcaaggccagcggctacaccttcaccagctacaacatgcactgggtcaagcagacccctggacagggactcgagtggatcggagccatctatcccggcaatggcgacacctcctacaaccagaagttcaagggcaaagccacactgaccgccgacaagagcagcagcacagcctacatgcagctgagcagcctgaccagcgaagatagcgccgtgtactactgcgctagagcccagctgaggcccaactactggtacttcgatgtgtggggagccggcaccaccgtgaccgtgtcaaaaatttctggcggcggaggatctggcggaggtggaagcggaggcggaggaagcggtggatcttctgacatcgtgctgtctcagagccccgccatcctttcagctagccctggcgagaaagtgaccatgacctgtagagccagcagcagcgtgtcctatatgcactggtatcagcagaagcccggcagcagccctaagccttggatctacgccacaagcaatctggccagcggagtgcctgccagattttctggctctggcagcggcacaagctactccctgacaatcagcagagtggaagccgaggatgccgccacctactactgtcagcagtggatcagcaaccctcctacctttggcgctggcaccaagctggaactgaagtctgcatggtcccatcctcagttcgagaagtctggaatgcatacaacaacccctgctcctcggcctcctacaccagctcctacaattgccagccagccactgtctctgaggcccgaagcttgtagacctgctgctggcggagccgtgcatacaagaggactggatttcgcctgcgacgtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgccttgggctgagcgaccacgagatagatcggctggagctacagaacgggcgctgcctgcgcgaggcgcaatacagtatgctggcgacctggaggcggcgcacgccacggcgcgaggccacgctggagctgctgggtcgcgtgctccgcgacatggacctgctgggctgcctggaggacatcgaggaggcgctttgcggccccgccgccctcccgcccgcgcccagtcttctcagaacgcgtagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagagctagcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 109 - protein, 950 aa, Artificial Sequence
  source: /note=“Description of Artificial Sequence Synthetic polypeptide”
  source: /note=“CD20-CAR (TNFR1TM-TNFR1-CD3z)-P2A-GFP”
MALPVTALLLPLALLLHAARPSQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSKISGGGGSGGGGSGGGGSGGSSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELKSAWSHPQFEKSGMHTTTPAPRPPTPAPTIASQPLSLRPEACRPAAGGAVHTRGLDFACDVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLRTRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 110 - protein, 214 aa, Artificial Sequence
  source: /note=“Description of Artificial Sequence Synthetic peptide”
  source: /note=“CD8 H-TNFR2 TM-TNFR2del151-3z”
TTTPAPRPPTPAPTIASQPLSLRPEACRPAAGGAVHTRGLDFACDFALPVGLIVGVTALGLLIIGVVNCVIMTQVKKKPLCLQREAKVPHLPADKARGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAS

SEQ ID NO: 111 - protein, 15 aa, Artificial Sequence
  source: /note=“Description of Artifical Sequence Synthetic peptide”
  source: ./note=“Linker”
GGGGSGGGGSGGGGS

SEQ ID NO: 112 - protein, 4 aa, Artificial Sequence
  source: /note=“Description of Artifical Sequence Synthetic peptide”
  source: ./note=“Linker”
GGGS

SEQ ID NO: 113 - protein, 16 aa, Artificial Sequence
  source: /note=“Description of Artifical Sequence Synthetic peptide”
  source: ./note=“Linker”
GGGSGGGSGGGSGGGS

SEQ ID NO: 114 - protein, 5 aa, Artificial Sequence
  source: /note=“Description of Artifical Sequence Synthetic peptide”
  source: ./note=“Linker”
GGGGS

SEQ ID NO: 115 - protein, 10 aa, Artificial Sequence
  source: /note=“Description of Artifical Sequence Synthetic peptide”
  source: ./note=“Linker”
GGGGSGGGGS

SEQ ID NO: 116 - protein, 15 aa, Artificial Sequence
  source: /note=“Description of Artifical Sequence Synthetic peptide”
  source: ./note=“Linker”
GGGGSGGGGSGGGGS

SEQ ID NO: 117 - protein, 20 aa, Artificial Sequence
  source: /note=“Description of Artifical Sequence Synthetic peptide”
  source: ./note=“Linker”
GGGGSGGGGSGGGGSGGGGS

(19) United States
(12) Patent Application Publication
(10) Pub. No.: US 2021/0292389 A1
(43) Pub. Date: Sep. 23, 2021

(54) NEW CAR CONSTRUCTS COMPRISING TNFR2 DOMAINS

(71) Applicant: SANGAMO THERAPEUTICS FRANCE, Valbonne Sophia Antipolis (FR)

(72) Inventors: Tobias Abel, Antibes (FR); David Fenard, Antibes (FR); Julie Gertner-Dardenne, Grasse (FR); François Meyer, Porrentruy (CH)

(73) Assignee: SANGAMO THERAPEUTICS FRANCE, Valbonne Sophia Antipolis (FR)

(21) Appl. No.: 17/267,410

(22) PCT Filed: Aug. 9, 2019
(86) PCT No.: PCT/IB2019/000907

§ 371 Date: Feb. 9, 2021

Related U.S. Application Data
(60) Provisional application No. 62/717,234, filed on Aug. 10, 2018.

(51) Int. Cl. C07K 14/705; C07K 14/725; C07K 16/28; A61K 35/17; A61P 37/06

(52) U.S. Cl. CPC C07K 14/70578; C07K 14/70517; C07K 14/70521; C07K 14/7051; C07K 16/2866; A61K 2039/5156; C07K 16/2887; C07K 16/2833; A61K 35/17; A61P 37/06; C07K 16/2803

(57) ABSTRACT

The present invention relates to a chimeric antigen receptor (CAR) comprising a human TNFR2 transmembrane domain (TM) or a fragment or variant thereof and/or a human TNFR2 costimulatory intracellular signaling domain or a fragment or variant thereof, and to an immune cell expressing said CAR. The present invention further relates to therapeutic methods comprising the administration of an immune cell expressing a chimeric antigen receptor (CAR) comprising a human TNFR2 transmembrane domain (TM) or a fragment or variant thereof and/or a human TNFR2 costimulatory intracellular signaling domain or a fragment or variant thereof.

### CROSS-REFERENCE TO RELATED APPLICATIONS

This application claims priority from U.S. Provisional Patent Application 62/717,234, filed Aug. 10, 2018. The disclosure of that application is incorporated by reference herein in its entirety.

### SEQUENCE LISTING

The instant application contains a Sequence Listing which has been submitted electronically in ASCII format and is hereby incorporated by reference in its entirety. The electronic copy of the Sequence Listing, created on Aug. 9, 2019, is named 025297_WO003_SL.txt and is 195,578 bytes in size.

### FIELD OF THE INVENTION

The present invention relates to the field of immunotherapy. In particular, the present invention relates to a chimeric antigen receptor (CAR) comprising a TNFR2 transmembrane domain (TM) or a fragment or variant thereof and/or a TNFR2 intracellular domain or a fragment or variant thereof. The present invention also relates to a cell population expressing said CAR and to the use thereof for treating diseases or disorders.

### BACKGROUND OF THE INVENTION

Chimeric antigen receptor (CAR) technology has recently revolutionized cancer therapy, especially in the context of B cell lymphoma and leukemia. While CAR engineered pro-inflammatory T cells are extensively studied and suggested to provide efficacy in the treatment of hematologic malignancies in early phase clinical trials, CAR engineered regulatory T cells (Tregs) are less evaluated.

Human Tregs play a key role in maintenance of immune homeostasis and thus may be used as therapeutic means in diverse clinical conditions. They also have potent immunosuppressive properties that can be harnessed to confer antigen-specific immunomodulation in a therapeutic setting. For these reasons, Treg cell therapy has been developed with the aim of treating, for example, chronic inflammatory diseases, autoimmune diseases, allergic diseases, and organ transplantation conditions such as graft rejection or graft-versus-host disease (GvHD).

In the art, the transduction of Treg cells with a CAR construct has been suggested, for example, in PCT Patent Publication WO 2008/095141.

Various molecular formats of CAR have been developed, differing in their extracellular, transmembrane, and cytoplasmic domains. In the T effector cell field, the intracellular module of the chimeric antigen receptor (CAR) typically consists of CD28, ICOS or 4-1BB domains in tandem with CD3 zeta. However, the use of these prototypical modules to design CAR Treg cells often leads to uncontrolled constitutive signaling that results in uncontrolled constitutive activation. This tonic signaling (corresponding to an antigen-independent background of activation) can lead to premature exhaustion of CAR Treg cells, thereby limiting their therapeutic use.

There is thus a need for new CAR constructs with lower tonic signaling when expressed in immune cells, particularly Treg cells.

### SUMMARY OF THE INVENTION

The present invention provides new CAR constructs comprising TNFR2 transmembrane domains or fragments or variants thereof and/or TNFR2 intracellular domains or fragments or variants thereof. As demonstrated herein, engineered T cells and engineered Treg cells expressing said CAR constructs present a strong decrease of tonic signaling as compared to said cells expressing a conventional CAR. Following CAR engagement, the engineered Treg cells showed highly efficient suppressive activity on T effector cell proliferation, thereby demonstrating the advantage of these Treg cells for cell therapy.

In some embodiments, the human Treg cells exhibit one or more of the following characteristics: a) compared to Tregs expressing a CAR having a human CD8 transmembrane domain and a 4-1BB costimulatory intracellular signaling domain, the present Treg cells express the CAR on the cell surface at a lower level and yet exhibit comparable levels of CAR-specific activation; b) the present Treg cells retain their Treg phenotype (e.g., high levels of expression of FoxP3, Helios, and CD62L, and low levels of expression of CD127) after more than one week (e.g., nine days of culture); and c) the present Treg cells are able to control GvHD in vivo (e.g., in a mouse GvHD model).

In some embodiments, the present invention provides a CAR comprising an extracellular binding domain, a transmembrane domain, and an intracellular domain, wherein

the transmembrane domain comprises a human tumor necrosis factor receptor 2 (TNFR2) transmembrane domain or a fragment or variant thereof, or

the intracellular domain comprises a human TNFR2 costimulatory intracellular signaling domain or a fragment or variant thereof, or

both (i) and (ii).

In certain embodiments, a CAR described herein further comprises an extracellular hinge domain, e.g., a hinge region of human CD8 or CD28. In particular embodiments, the hinge domain comprises the sequence of SEQ ID NO: 14 or a sequence having at least about 70% identity with SEQ ID NO: 14.

In certain embodiments, the intracellular domain of a CAR described herein comprises an immune cell primary intracellular signaling domain, e.g., a T cell primary intracellular signaling domain of human CD3. In particular embodiments, the intracellular domain comprises a primary intracellular signaling domain of human CD3 zeta, optionally comprising the sequence of SEQ ID NO: 28, 29, 30 or 31 or a sequence having at least about 70% identity with SEQ ID NO: 28, 29, 30 or 31.

In certain embodiments, the CAR comprises:

an extracellular binding domain,

an extracellular hinge domain comprising a hinge region of human CD8 or CD28,

a transmembrane domain comprising a human TNFR2 transmembrane domain or a fragment or variant thereof, and

an intracellular domain comprising a primary intracellular signaling domain of human CD3 zeta.

In certain embodiments, the CAR comprises:

an extracellular binding domain,

an extracellular hinge domain comprising a hinge region of human CD8 or CD28, a transmembrane domain, and

an intracellular domain comprising a human TNFR2 costimulatory intracellular signaling domain or a fragment or variant thereof, and a primary intracellular signaling domain of human CD3 zeta.

In certain embodiments, the CAR comprises:

an extracellular binding domain,

an extracellular hinge domain comprising a hinge region of human CD8 or CD28,

a transmembrane domain comprising a human TNFR2 transmembrane domain or a fragment or variant thereof, and

an intracellular domain comprising a human TNFR2 costimulatory intracellular signaling domain or a fragment or variant thereof, and a primary intracellular signaling domain of human CD3 zeta.

In certain embodiments, the transmembrane domain of a CAR described herein comprises at least eight contiguous amino acids of SEQ ID NO: 22 or of a sequence having at least about 70% identity with SEQ ID NO: 22. In certain embodiments, the transmembrane domain comprises at least eight contiguous amino acid residues of SEQ ID NO: 22 in combination with amino acid residues from a transmembrane domain of a protein that is not TNFR2. In certain embodiments, the transmembrane domain comprises the amino acid sequence of VNCVIMTQV (SEQ ID NO: 63).

In certain embodiments, the intracellular domain of a CAR described herein comprises at least 30 contiguous amino acid residues of SEQ ID NO: 34 or of a sequence having at least about 70% identity with SEQ ID NO: 34. In certain embodiments, the intracellular domain comprises at least 30 contiguous amino acid residues of SEQ ID NO: 34 in combination with amino acid residues from a costimulatory intracellular signaling domain of a protein that is not TNFR2. In certain embodiments, the intracellular signaling domain comprises residues 1-70, 1-115, or 1-156 of SEQ ID NO: 34.

In certain embodiments, the CAR comprises:

an extracellular binding domain,

an extracellular hinge domain comprising a CD8 hinge region of SEQ ID NO: 14,

a transmembrane domain comprising a TNFR2 transmembrane domain of SEQ ID NO: 22, and

an intracellular domain comprising:
a primary human CD3 zeta intracellular signaling domain of SEQ ID NO: 28, 29, 30, or 31, and
a TNFR2 costimulatory intracellular signaling domain of SEQ ID NO: 34.

In certain embodiments, the extracellular binding domain of a CAR described herein is an antibody or an antigen-binding fragment thereof. In particular embodiments, the extracellular binding domain is a single chain variable fragment (scFv). The extracellular binding domain may specifically bind, e.g.,

an autoantigen, wherein the autoantigen is optionally IL-23 receptor (IL-23R);

a B cell antigen, optionally selected from CD19 and CD20; or

an allogeneic HLA class I or class II molecule, wherein the class I molecule is optionally HLA-A2.

The present invention also provides a nucleic acid sequence encoding a CAR described herein, as well as a vector comprising the nucleic acid sequence and a host cell comprising the nucleic acid sequence or the vector.

The present invention also provides a population of immune cells expressing a CAR described herein. In some embodiments, the immune cells are selected from the group consisting of T cells, natural killer (NK) cells, αβ T cells, γδ T cells, double negative (DN) cells, regulatory immune cells, regulatory T (Treg) cells, effector immune cells, effector T cells, B cells, and myeloid-derived cells, and any combination thereof, wherein the immune cells are optionally human cells. In particular embodiments, the population comprises Treg cells, wherein the Treg cells are optionally human cells.

In certain embodiments, the immune cell population comprises human Treg cells expressing a CAR comprising:

an extracellular binding domain,

a hinge domain comprising a hinge region of human CD8,

a human TNFR2 transmembrane domain, and

an intracellular domain comprising a human TNFR2 costimulatory intracellular signaling domain and a primary intracellular signaling domain of human CD3 zeta.

The present invention also provides a pharmaceutical composition comprising an immune cell, a host cell, or an immune cell population expressing a CAR described herein, and a pharmaceutically acceptable excipient. Also provided is a method for treating a disease or disorder in a human subject in need thereof, comprising administering to the subject the pharmaceutical composition.

The present invention also provides a chimeric antigen receptor or immune cell population described herein, for use in the treatment of a disease or disorder in a human subject in need thereof.

The present invention also provides the use of a chimeric antigen receptor or immune cell population described herein for the manufacture of a medicament for the treatment of a disease or disorder in a human subject in need thereof.

In some embodiments, the disease or disorder is selected from the group consisting of an inflammatory disease, an autoimmune disease, an allergic disease, an organ transplantation condition, a cancer, and an infectious disease.

In some embodiments, the human subject is in need of immunosuppression and the CAR is expressed in Treg cells in the human subject.

In some embodiments, the disease or disorder is an inflammatory disease, an autoimmune disease, an allergic disease, or an organ transplantation condition (e.g., graft rejection or graft-versus-host disease).

The invention also provides a chimeric antigen receptor or immune cell population described herein for use as a medicament.

### BRIEF DESCRIPTION OF THE DRAWINGS

FIG. 1 depicts a schematic view of CD19-CAR, CD20-CAR, and IL-23R-CAR constructs. The CARs comprise a human CD8 leader sequence (CD8), optionally a hemagglutinin tag (HA), an scFv sequence (anti-CD19, anti-CD20, or anti-IL-23R), optionally a streptavidin tag (Tag), a hinge domain (linker), a transmembrane domain (TNFR2 or CD8), a costimulatory intracellular signaling domain (4-IBB or TNFR2) and CD3 zeta (CD3ζ). The CAR construct is in frame with a P2A-GFP coding sequence.

FIG. 2 depicts flow cytometry dot plots showing transduction efficiency and CAR expression at the cell surface of Tregs. Transduction efficiency was assessed by GFP expression and CAR expression was assessed by HA expression for CD19-CAR (CD8TM/4-1BB or TNFR2) or protein L staining for CD20-CAR (CD8TM/4-1BB or TNFR2). MFI: Mean Fluorescence Intensity.

FIG. 3 shows the Western blot analysis of CAR expression in human Tregs transduced with a CD20-CAR (CD8TM/4-1BB or TNFR2) or untransduced (“Blank”). Staining with CD3 zeta specific antibody revealed the endogenous CD3 zeta at 16 kD, the CD20-CAR (CD8TM/4-1BB) at ˜62 kD, and the CD20-CAR (TNFR2) at 82 kD (Panel A, upper left). The membrane was then washed and reprobed with β-actin antibody as a loading control (Panel A, lower left). Band intensity was quantified using Image J and results are shown from two different donors in Panel B.

FIG. 4 depicts histograms showing that TNFR2-derived CARs maintain CAR-specific activation. At Day 9, transduced FoxP3 Tregs were seeded alone or in the presence of anti-CD3/anti-CD28 coated beads, or in the presence of freshly thawed autologous B cells. After 24 h, CD19-CARs (upper left), CD20-CARs (upper right), and IL-23R-CARs (lower) were stained for CD4 and CD69 cell surface expression. Error bars represent mean±SEM. CTRL: Treg cells not transduced with a CAR.

FIG. 5 is a graph showing that TNFR2-derived CARs exhibit efficient CAR-mediated suppressive activity. Contact-dependent suppression mediated by CD19-CAR Tregs (Panel A), CD20-CAR Tregs (Panel B), or IL-23R-CAR Tregs (Panel C) in the absence of any activation (dotted lines) or after B cell-induced CAR activation (solid lines) was evaluated by measuring the proliferation of conventional T cells (Tconv) using flow cytometry. Circle lines represent CD8TM/4-1BB CAR constructs and square lines represent TNFR2 CAR constructs. Error bars represent mean±SEM.

FIG. 6 depicts a schematic view of CD19-CAR constructs of the invention. The CAR comprises a human CD8 leader sequence (CD8), an scFv sequence (anti-CD19), a streptavidin tag (Tag), a hinge domain (linker), a transmembrane domain (CD8, TNFR2 or fused CD8/TNFR2), a costimulatory intracellular signaling domain (4-IBB, TNFR2 or TNFR2 fragments) and CD3 zeta (CD3z). The CAR construct is in frame with a P2A-GFP coding sequence.

FIG. 7 is a pair of graphs showing that TNFR2-C-terminal deletion constructs exhibit different surface expression levels and are functional in CD3z signaling in Jurkat-NFAT cells. Jurkat-NFAT cells were transduced with the indicated constructs. After one week in culture, CAR surface expression was determined by protein staining (Panel A), and cells were activated by CD19-expressing Daudi cells in a 1:1 ratio. 24 hours later, NFAT-depended luciferase secretion was determined using a Glowmax luminometer (Panel B).

FIG. 8 is a set of dot plots showing transduction efficiency and CAR expression at the cell surface (top and bottom left) and a graph showing the viability of the transduced CAR-Treg cells (bottom right). Transduction efficiency at Day 8(%) was assessed using GFP expression levels, and CAR density (MFI) was assessed using protein-L labeling. Cell viability was evaluated using the propidium iodide exclusion method. Error bars represent mean±SD.

FIG. 9 is a graph showing ligand-independent tonic signaling and activation capacity of anti-CD20 CARs. At Day 9, transduced FoxP3 Tregs were seeded alone (“None”), in the presence of anti-CD3/anti-CD28 coated beads, or in the presence of freshly thawed autologous B cells (“B cells”). After 24 hours, cells were stained for CD4 and CD69 cell surface expression. Error bars represent mean±SD. For the “None” condition, a statistical analysis has been performed using the GFP condition as a control (*p<0.05, **p<0.01 and ***p<0.001, Paired T-test).

FIG. 10 depicts a set of graphs showing that TNFR2-derived CD20 CARs exhibit efficient CAR-mediated suppressive activity, but not 4-1BB and TNFR1-derived CD20 CARs. Contact-dependent suppression mediated by CAR Treg cells in the absence of any activation (“None”) or after B cell-induced CAR activation (“B cells”) was evaluated by measuring the proliferation of conventional T cells (Tconv).

FIG. 11 is a set of graphs showing potency of CAR-mediated suppressive activity. Contact-dependent suppression (%) after B-cell induced CAR activation using TNFR2-derived (top), or TNFR1-derived (bottom) CARs was represented as a function of the number of CAR-Treg cells in the assay. This representation allows the calculation of the number of CAR-Tregs necessary to trigger 50% suppression.

FIG. 12 depicts a schematic view of HLA-A2-CAR constructs used in Example 5. The CARs comprise a human CD8 leader sequence (CD8), an anti-HLA-A2 scFv sequence, a hinge domain (linker), a transmembrane domain (TNFR2 or CD8 TM), a cosignaling domain (CD28 or TNFR2 or TNFR2+4-1BB) and CD3 zeta (CD3Z). These CAR constructs are in frame with a P2A-GFP coding sequence.

FIG. 13 depicts flow cytometry dot plots showing transduction efficiency and CAR expression at the cell surface of Tregs. Transduction efficiency was assessed by GFP expression and CAR expression at the cell surface was assessed by Dextramer® expression.

FIG. 14 depicts flow cytometry dot plots showing the presence of Treg phenotypic markers on HLA*A2 CAR-Tregs.

FIG. 15 is a set of graphs showing body weight variation (top left), GvHD score (top right), and percent survival free disease (bottom) over time for NSG mice injected with HLA*A2-CAR-Tregs comprising TNFR2, CD28, or TNFR2+4-1BB domains.

### DETAILED DESCRIPTION OF THE INVENTION

### Definitions

In the present invention, the following terms have the following meanings:

The terms “a” and “an” refer to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “activation” as used herein, refers to the state of a T cell (e.g., a regulatory T cell) that has been sufficiently stimulated to induce a detectable cellular response. Activation can also be associated with detectable effector function(s) such as cytokine production or suppressive activity. The term “activated” regulatory T cells refers to, among other things, regulatory T cells that are capable of suppressing an immune response.

The term “affibody” is well known in the art and refers to affinity proteins based on a 58 amino acid residue protein domain, derived from one of the IgG binding domains of staphylococcal protein A.

The term “allogeneic” refers to any material derived from a different individual of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “antibody” or “immunoglobulin” (Ig), as used herein, refers to a protein or polypeptide sequence derived from an immunoglobulin molecule that specifically binds with an antigen. Antibodies can be polyclonal or monoclonal, multiple or single chain, or intact immunoglobulins, and may be derived from natural sources or from recombinant sources. The term “antibody” also includes multispecific antibodies (e.g., bispecific antibodies) and antibody fragments, so long as they exhibit the desired biological activity. Antibodies can be multimers of immunoglobulin molecules, such as tetramers of immunoglobulin molecules.

The basic four-chain antibody unit is a heterotetrameric glycoprotein composed of two identical light (L) chains and two identical heavy (H) chains. The L chain from any vertebrate species can be assigned to one of two clearly distinct types, called kappa (κ) and lambda (λ), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in CH sequence and function, e.g., humans express the following subclasses: IgG1, IgG2, IgG3, IgG4, IgA1, and IgA2. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. An IgM antibody consists of five of the basic heterotetramer units along with an additional polypeptide called a J chain, and therefore, contains ten antigen binding sites, while secreted IgA antibodies can polymerize to form polyvalent assemblages comprising 2-5 of the basic 4-chain units along with J chain. In the case of IgGs, the 4-chain unit is generally about 150,000 Daltons. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The term “monoclonal antibody” as used herein refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, a monoclonal antibody may be prepared by the hybridoma methodology first described by Kohler et al., Nature 256:495 (1975), or may be made using recombinant DNA methods in bacterial, eukaryotic animal or plant cells (see, e.g., U.S. Pat. No. 4,816,567). A “monoclonal antibody” may also be isolated from phage antibody libraries using the techniques described in Clackson et al., Nature 352:624-628 (1991) and Marks et al., J. Mol. Biol. 222:581-597 (1991), for example. The monoclonal antibodies described herein include “chimeric” antibodies, which comprise one or more regions from one antibody (e.g., non-human variable domains) and one or more regions from one or more other antibodies (e.g., human constant regions).

The term “antibody fragment” refers to at least one portion of an intact antibody, e.g., the antigen binding region or variable region of the intact antibody, that retains the ability to specifically interact with (e.g., by binding, steric hindrance, stabilizing/destabilizing, and/or spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136 (2005)). Antigen binding fragments can also be grafted into scaffolds based on polypeptides such as a fibronectin type III (see, e.g., U.S. Pat. No. 6,703,199, which describes fibronectin polypeptide minibodies). Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of crosslinking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

An “intact antibody” is one that comprises an antigen-binding site as well as a CL and at least heavy chain constant domains CH1, CH2 and CH3. The constant domains may be native sequence constant domains (e.g., human native sequence constant domains) or amino acid sequence variants thereof. A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

As used herein, a “functional fragment or analog of an antibody” is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, FcεRI.

The term “antibody heavy chain” refers to the larger of the two types of polypeptide chains present in antibody molecules in their naturally occurring conformations, and which normally determines the class to which the antibody belongs.

The term “antibody light chain” refers to the smaller of the two types of polypeptide chains present in antibody molecules in their naturally occurring conformations. Kappa (κ) and lambda (λ) light chains refer to the two major antibody light chain isotypes.

“Anticalins” are well known in the art and refer to an antibody mimetic technology, wherein the binding specificity is derived from lipocalins. Anticalins may also be formatted as dual targeting proteins, called Duocalins.

The term “antigen” or “Ag” refers to a molecule that provokes an immune response. This immune response may involve antibody production, the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response therefore encodes an “antigen” as that term is used herein. Furthermore, one skilled in the art will understand that an antigen does not necessarily need to be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen does not necessarily need to be encoded by a “gene” at all. It is readily apparent that an antigen can be synthesized or can be derived from a biological sample, or might be a macromolecule besides a polypeptide. Such a biological sample can include, but is not limited to, e.g., a tissue sample, a cell or a fluid with other biological components.

The term “antigen presenting cell” or “APC” refers to an immune system cell such as an accessory cell (e.g., a B cell, a dendritic cell, and the like) that displays a foreign antigen complexed with major histocompatibility complexes (MHCs) on its surface. T cells may recognize these complexes using their T cell receptors (TCRs). APCs process antigens and present them to T cells.

The term “autologous” refers to any material derived from the same individual to whom it is later to be re-introduced.

“Avimers” are well known in the art and refer to an antibody mimetic technology.

The term “chimeric receptor” or “chimeric antigen receptor” or “CR” or “CAR” refers to one polypeptide or to a set of polypeptides, typically two in the simplest embodiments, which when in an immune cell, provides the cell with specificity for a target ligand and with intracellular signal generation. In some embodiments, the set of polypeptides are contiguous with each other. In some embodiments, the chimeric receptor is a chimeric fusion protein comprising the set of polypeptides. In some embodiments, the set of polypeptides includes a dimerization switch that, upon the presence of a dimerization molecule, can couple the polypeptides to one another, e.g., can couple a ligand binding domain to an intracellular signaling domain. In some embodiments, the chimeric receptor comprises an optional leader sequence at the amino-terminus (N-ter) of the chimeric receptor fusion protein. In some embodiments, the chimeric receptor comprises a leader sequence at the N-terminus of the extracellular ligand binding domain, wherein the leader sequence is optionally cleaved from the ligand binding domain during cellular processing and localization of the chimeric receptor to the cellular membrane.

The term “conservative sequence modifications” refers to amino acid modifications that do not significantly affect or alter the biological function of the protein containing the amino acid sequence. Such conservative modifications include amino acid substitutions, additions and deletions. Modifications can be introduced into a protein by standard techniques known in the art, such as site-directed mutagenesis and PCR-mediated mutagenesis. “Conservative amino acid substitutions” are ones in which the amino acid residue is replaced with an amino acid residue that has similar properties, such that one skilled in the art of peptide chemistry would expect the secondary structure and hydropathic nature of the polypeptide to be substantially unchanged. Amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Other families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a chimeric receptor of the invention can be replaced with other amino acid residues from the same side chain family and the altered chimeric receptor can be tested using the functional assays described herein.

The term “constitutive promoter” refers to a nucleotide sequence that, when operably linked with a polynucleotide that encodes or specifies a gene product, causes the gene product to be produced in a cell under most or all physiological conditions of the cell.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. A costimulatory signaling domain can be the intracellular portion of a costimulatory molecule. A costimulatory molecule can be represented in the following protein families: TNF receptor proteins, immunoglobulin-like proteins, cytokine receptors, integrins, signaling lymphocytic activation molecules (SLAM proteins), and activating NK cell receptors.

A “cytotoxic cell” includes any cell capable of mediating a cytotoxicity response.

The term “derived from,” as used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3 zeta molecule, the intracellular signaling domain retains sufficient CD3 zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3 zeta sequence and delete unwanted sequence, or impose mutations, to arrive at the intracellular signaling domain.

The term “diabodies” refers to small antibody fragments prepared by constructing sFv fragments with short linkers (about 5-10 residues) between the VH and VL domains such that inter-chain but not intra-chain pairing of the V domains is achieved, resulting in a bivalent fragment, i.e., a fragment having two antigen binding sites. Bispecific diabodies are heterodimers of two “crossover” sFv fragments in which the VH and VL domains of the two antibodies are present on different polypeptide chains. Diabodies are described more fully in, for example, EP 0404097; WO 93/11161; and Holliger et al., Proc. Natl. Acad. Sci. USA, 90:6444-6448 (1993).

A “domain antibody” is well known in the art and refers to the smallest functional binding unit of an antibody, corresponding to the variable region of either the heavy or light chain of an antibody.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some version contain an intron(s).

The term “endogenous” refers to any material naturally from or naturally produced inside an organism, cell, tissue or system.

The term “engineered” or “modified” refers to a cell that has been transfected, transformed or transduced.

The term “exogenous” refers to any material introduced to or produced outside an organism, cell, tissue or system.

The term “expression” refers to the transcription and/or translation of a particular nucleotide sequence driven by a promoter.

The term “expression vector” refers to a vector comprising a recombinant polynucleotide comprising an expression control sequence operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes), transposons (e.g., sleeping beauty) and viruses (e.g., lentiviruses, retroviruses, adenoviruses, and adeno-associated viruses) that incorporate the recombinant polynucleotide.

The term polynucleotide “fragment” as used herein, is a polynucleotide that typically differs from a polynucleotide specifically disclosed herein in one or more deletions. Such fragments may be naturally occurring or may be synthetically generated, for example, by modifying one or more of the polynucleotide sequences of the invention and evaluating one or more biological activities of the encoded fragment as described herein and/or by using any of a number of techniques well known in the art. Accordingly, the term polypeptide “fragment” as used herein, is a polypeptide that typically differs from a polypeptide specifically disclosed herein in one or more deletions. Such fragments may be naturally occurring or may be synthetically generated, for example, by modifying one or more of the polypeptide sequences of the invention and evaluating one or more biological activities of the polypeptide as described herein and/or by using any of a number of techniques well known in the art. Modifications may be made in the structure of the polynucleotides and polypeptides of the present invention and still result in a functional molecule that encodes or is a fragment polypeptide with desirable characteristics and without appreciable loss of biological utility or activity. In some embodiments, polypeptide fragments differ from a native sequence by deletion of less than 50, 40, 30, 20, 10, 9, 8, 7, 6, 5, 4, 3, 2, or 1 amino acid. Fragments may also (or alternatively) be modified by, for example, the deletion of amino acids that have minimal influence on the immunogenicity, secondary structure and hydropathic nature of the polypeptide.

The “Fe” fragment of an antibody comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) may have the ability to recognize and bind the antigen, although at a lower affinity than the entire binding site.

“Framework” or “FR” residues are those variable domain residues other than the hypervariable region residues herein defined.

The term “graft-versus-host disease” or “GVHD” as used herein refers to a medical complication following the receipt of transplanted tissue from a genetically different person. Immune cells in the donated tissue (the graft) recognize the recipient (the host) as foreign. The transplanted immune cells then attack the host's body cells. GVHD is commonly associated with stem cell transplant; however, the term includes GVHD arising from other forms of tissue graft. GVHD may also occur after a blood transfusion.

The term “homology” or “identity” refers to the subunit sequence identity between two polymeric molecules, e.g., between two nucleic acid molecules, such as two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Thus, the term “homologous” or “identical,” when used in a relationship between the sequences of two or more polypeptides or of two or more nucleic acid molecules, refers to the degree of sequence relatedness between polypeptides or nucleic acid molecules, as determined by the number of matches between strings of two or more amino acid or nucleotide residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48:1073 (1988). Preferred methods for determining identity are designed to give the largest match between the sequences tested. Methods of determining identity are described in publicly available computer programs. Exemplary computer program methods for determining identity between two sequences include the GCG program package, including GAP (Devereux et al., Nucl. Acid. Res. 12:387 (1984); Genetics Computer Group, University of Wisconsin, Madison, Wis.), BLASTP, BLASTN, and FASTA (Altschul et al., J. Mol. Biol. 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

The term “humanized” as it relates to forms of non-human (e.g., murine) antibodies refers to chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulins. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues that are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see, e.g., Jones et al., Nature 321:522-525 (1986); Reichmann et al., Nature, 332:323-329 (1988); Presta, Curr. Op. Struct. Biol. 2:593-596 (1992).

As used herein, the term “immune cells” generally includes white blood cells (leukocytes) that are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, and dendritic cells).

As used herein, the term “immune effector cell” refers to a cell of the immune system that is in a form that is capable of mounting a specific immune response.

As used herein, the term “immune regulatory cell” refers to an immune cell that acts in a “regulatory” way to suppress activation of the immune system and thereby maintains immune system homeostasis and tolerance to self-antigens. “Regulatory immune cells” may also have effects on non-immune cells that result in an improved clinical state such as promoting tissue repair or regeneration. Regulatory immune cells may include, without limitation, regulatory T cells (e.g., CD4⁺ regulatory T cells, CD8⁺ regulatory T cells, regulatory γδ T cells, and/or regulatory DN T cells), regulatory B cells, regulatory NK cells, regulatory macrophages, and regulatory dendritic cells.

As used herein, the term “immune response” includes T cell mediated and/or B cell mediated immune responses. Exemplary immune responses include T cell responses, e.g., proliferation, cytokine production and cellular cytotoxicity. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Thl and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; and myeloid cells, such as macrophages, eosinophils, mast cells, basophils, and granulocytes.

As used herein, the term “immune accommodation” refers to a condition of a transplant recipient in which an organ or tissue transplant functions normally despite the presence of antibodies in the recipient that are specific for the organ or tissue transplant.

As used herein, the term “immunological tolerance” or “immune tolerance” refers to a) a decreased level of a specific immunological response (thought to be mediated at least in part by antigen-specific effector T lymphocytes, B lymphocytes, antibody, or their equivalents); b) a delay in the onset or progression of a specific immunological response; or c) a reduced risk of the onset or progression of a specific immunological response, in one population of subjects (e.g., subjects that have undergone a treatment, such as a treatment described herein) in comparison with a different population of subjects (e.g., subjects that have not undergone the treatment). “Specific” immunological or immune tolerance occurs when immunological or immune tolerance is preferentially invoked against certain antigens in comparison with others.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

The term “inducible” promoter refers to a nucleotide sequence that, when operably linked with a polynucleotide that encodes or specifies a gene product, causes the gene product to be produced in a cell substantially only when an inducer that corresponds to the promoter is present in the cell.

As used herein, a “5′ cap” (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group that is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

In the context of the present invention, the following abbreviations for the commonly occurring nucleic acid bases are used. “A” refers to adenine, “C” refers to cytosine, “G” refers to guanine, “T” refers to thymine, and “U” refers to uracil.

The term “instructional material” includes a publication, a recording, a diagram, or any other medium of expression that can be used to communicate the usefulness or use of the compositions and methods of the invention. The instructional material of the kit of the invention may, for example, be affixed to a container that contains a nucleic acid, vector, cell population, or composition of the invention or be shipped together with a container that contains a nucleic acid, vector, cell population, or composition of the invention. Alternatively, the instructional material may be shipped separately from the container with the intention that the instructional material cell be used cooperatively by the recipient.

The term “intracellular signaling domain,” as used herein, refers to an intracellular portion of a molecule. The intracellular signaling domain generates a signal that promotes an immune effector function of the chimeric receptor containing cell. Examples of immune effector function in a chimeric receptor-T cell may include cytolytic activity, suppressive activity, regulatory activity and helper activity, including the secretion of cytokines.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated”. An isolated nucleic acid or peptide can exist in substantially purified form, or can exist in a non-native environment such as, for example, a host cell. Typically, a preparation of isolated nucleic acid or peptide contains the nucleic acid or peptide at least about 80% pure, at least about 85% pure, at least about 90% pure, at least about 95% pure, greater than 95% pure, greater than about 96% pure, greater than about 97% pure, greater than about 98% pure, or greater than about 99% pure. An “isolated polypeptide” is one that has been identified and separated and/or recovered from a component of its natural environment.

An “isolated nucleic acid” or “isolated nucleic sequence” is a nucleic acid that is substantially separated from other genome DNA sequences as well as proteins or complexes such as ribosomes and polymerases that naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man. In some embodiments, an isolated nucleic acid or isolated nucleic sequence does not occur in nature.

An “isolated polypeptide” is one that has been identified and separated and/or recovered from a component of its natural environment. In certain embodiments, the isolated polypeptide will be purified (1) to greater than 95% by weight of polypeptide as determined by the Lowry method, and in particular embodiments to more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, in certain embodiments, silver staining. Isolated polypeptide includes the polypeptide in situ within recombinant cells since at least one component of the polypeptide's natural environment will not be present. In certain embodiments, isolated polypeptide will be prepared by at least one purification step. In some embodiments, an isolated polypeptide does not occur in nature.

The term “lentivirus” refers to a genus of the Retroviridae family. Lentiviruses are unique among the retroviruses in being able to infect non-dividing cells. Because they can deliver a significant amount of genetic information into the DNA of the host cell, they are one of the most efficient gene delivery vectors. HIV, SIV, and FIV are all examples of lentiviruses.

The term “lentiviral vector” refers to a vector derived from at least a portion of a lentivirus genome, including, e.g., a self-inactivating lentiviral vector as provided in Milone et al., Mol. Ther. 17(8):1453-1464 (2009). Other examples of lentiviral vectors that may be used in the clinic include, but are not limited to, LENTIVECTOR® gene delivery technology from Oxford BioMedica and the LENTIMAX™ vector system from Lentigen. Nonclinical types of lentiviral vectors are also available and would be known to one skilled in the art.

The term “ligand” refers to a member of a ligand/receptor pair, and binds to the other member (receptor) of the pair.

The term “nucleic acid” or “polynucleotide” refers to a polymer of nucleotides covalently linked by phosphodiester bonds, such as deoxyribonucleic acids (DNA) or ribonucleic acids (RNA), in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

A “nanobody” is well known in the art and refers to an antibody-derived therapeutic protein that contains the unique structural and functional properties of naturally-occurring heavy chain antibodies. These heavy chain antibodies contain a single variable domain (VHH) and two constant domains (CH2 and CH3).

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins, among others. A polypeptide includes a natural peptide, a recombinant peptide, or a combination thereof.

The term “pharmaceutically acceptable excipient” or “pharmaceutically acceptable carrier” refers to an excipient that does not produce an adverse, allergic or other untoward reaction when administered to an animal, e.g., a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, the FDA Office or EMA

The term “poly(A)” refers to a series of adenosines monophosphate attached to the mRNA. In some embodiments of a construct for transient expression, the polyA is between 50 and 5000 adenosines monophosphate, e.g., greater than or equal to 64, greater than or equal to 100, or greater than or equal to 300 or 400 adenosines monophosphate. Poly(A) sequences can be modified chemically or enzymatically to modulate mRNA functionality such as localization, stability or efficiency of translation.

The term “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly (A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

The term “promoter” refers to a DNA sequence recognized by the synthetic machinery of a cell, or introduced synthetic machinery, required to initiate the specific transcription of a polynucleotide sequence.

The term “promoter/regulatory sequence” refers to a nucleic acid sequence that is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may, for example, be one that expresses the gene product in a tissue specific manner.

The term “recombinant protein or peptide” refers to a protein or peptide (e.g., an antibody) that is generated using recombinant DNA technology, such as, for example, a protein or peptide (e.g., an antibody) expressed by a bacteriophage or yeast expression system. The term should also be construed to mean a protein or peptide (e.g., an antibody) that has been generated by the synthesis of a DNA molecule encoding the protein or peptide (e.g., the antibody) wherein the DNA molecule expresses a protein or peptide (e.g., an antibody), or an amino acid sequence specifying the protein or peptide (e.g., the antibody), wherein the DNA or amino acid sequence has been obtained using recombinant DNA or amino acid sequence technology which is available and well known in the art.

The terms “regulatory T lymphocyte,” “regulatory T cell,” “T regulatory cell,” “Treg cell,” and “Treg” as used in the present invention are synonymous and are intended to have the standard definition as used in the art. Treg cells are a specialized subpopulation of T cells that act in a “regulatory” way to suppress activation of the immune system and thereby maintain immune system homeostasis and tolerance to self-antigens. Tregs have sometimes been referred to as suppressor T cells. Treg cells are often, but not always, characterized by expression of the forkhead family transcription factor FoxP3 (forkhead box P3). They may also express CD4 or CD8 surface proteins. They usually also express CD25. Tregs often are marked by the phenotype of CD4⁺CD25⁺CD127^{lo}FoxP3⁺. In some embodiments, Tregs are also CD45RA⁺, CD62L^{hi} and/or GITR⁺. In particular embodiments, Tregs are marked by CD4⁺CD25⁺CD127^{lo}CD62L⁺ or CD4⁺CD45RA⁺CD25^{hi}CD127^{lo}. As used in the present invention, and unless otherwise specified, Tregs include “natural” Tregs that develop in the thymus, induced/adaptive/peripheral Tregs that arise via a differentiation process that takes place outside the thymus (e.g., in tissues or secondary lymphoid organs, or in the laboratory setting under defined culture conditions), and Tregs that have been created using recombinant DNA technology. Naturally-occurring Treg cells (CD4⁺CD25⁺FoxP3⁺) arise like all other T cells in the thymus. In contrast, induced/adaptive/peripheral Treg cells (which include CD4⁺CD25⁺FoxP3⁺ Tregs, Tr1 cells, Th3 cells and others) arise outside the thymus. One way to induce Tregs is by exposure of T effector cells to IL-10 or TGF-β. T cells may also be converted to Treg cells by transfection or transduction of the FoxP3 gene into a mixed population of T cells. A T cell that is induced to express FoxP3 adopts the Treg phenotype and such recombinant Tregs are also defined herein as “Tregs”.

The term “rejection” refers to a state in which a transplanted organ or tissue is not accepted by the body of the recipient. Rejection results from the recipient's immune system attacking the transplanted organ or tissue. Rejection can occur days to weeks after transplantation (acute) or months to years after transplantation (chronic).

As used herein, an antibody or a CAR is said to be “immunospecific for,” “specific for” or to “specifically bind” an antigen if it reacts at a detectable level with the antigen, e.g., with an affinity constant, Ka, of greater than or equal to about 10⁴M⁻¹, greater than or equal to about 10⁵M⁻¹, greater than or equal to about 10⁶M⁻¹, greater than or equal to about 10⁷ M⁻¹, greater than or equal to 10⁸ M⁻¹, greater than or equal to 10⁹ M⁻¹, or greater than or equal to 10¹⁰ M⁻¹. Affinity of an antibody for its cognate antigen is also commonly expressed as a dissociation constant Kd, and in certain embodiments, an antibody specifically binds to antigen if it binds with a Kd of less than or equal to 10⁻⁴ M, less than or equal to about 10⁻⁵ M, less than or equal to about 10⁻⁶M, less than or equal to 10⁻⁷ M, less than or equal to 10⁻⁸ M, less than or equal to 5×10⁻⁹ M, or less than or equal to 10⁻⁹ M, or less than or equal to 5×10⁻¹⁰ M, or less than or equal to 10⁻¹⁰ M. Affinities of antibodies or CARs can readily be determined using conventional techniques, for example, those described by Scatchard et al., (Ann. N.Y. Acad. Sci. USA 51:660 (1949)). Binding properties of an antibody to antigens, cells or tissues thereof may generally be determined and assessed using immunodetection methods including, for example, immunofluorescence-based assays, such as immunohistochemistry (IHC) and/or fluorescence-activated cell sorting (FACS). In some embodiments, the term “specifically binds” refers to an antibody, a CAR or a ligand, that recognizes and binds with a binding partner present in a sample, but that does not substantially recognize or bind other molecules in the sample.

The term “signal transduction pathway” refers to the biochemical relationship between a variety of signal transduction molecules that play a role in the transmission of a signal from one portion of a cell to another portion of a cell.

The term “signaling domain” refers to a functional portion of a protein that acts by transmitting information within the cell to regulate cellular activity via defined signaling pathways by generating second messengers or functioning as effectors by responding to such messengers.

As used herein, the term “stem cell” generally includes pluripotent or multipotent stem cells. “Stem cells” include, without limitation, embryonic stem cells (ES); mesenchymal stem cells (MSC); induced-pluripotent stem cells (iPS); and committed progenitor cells (hematopoeitic stem cells (HSC), bone marrow derived cells, etc.).

The term “stimulation” refers to a primary response induced by binding of a stimulatory molecule (e.g., a TCR/CD3 complex or chimeric receptor) with its cognate ligand thereby mediating a signal transduction event, such as, but not limited to, signal transduction via the TCR/CD3 complex or signal transduction via signaling domains of the chimeric receptor. Stimulation can mediate altered expression of certain molecules.

The term “stimulatory molecule” refers to a molecule expressed by an immune cell (e.g., T cell, NK cell, or B cell) that provides a cytoplasmic signaling sequence(s) that regulates activation of the immune cell in a stimulatory way in at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, suppression, and the like. A primary cytoplasmic signaling sequence (also referred to as a “primary signaling domain”) that acts in a stimulatory manner may contain a signaling motif that is known as immunoreceptor tyrosine-based activation motif or ITAM.

The term “subject” is intended to include living organisms in which an immune response can be elicited (e.g., mammals such as humans). In some embodiments, a subject may be a “patient,” i.e, a warm-blooded animal such as a human, who is awaiting the receipt of or is receiving medical care or was/is/will be the object of, a medical procedure, or is monitored for the development of the targeted disease or condition, such as, for example, an inflammatory or autoimmune condition. In some embodiments, the subject is an adult (for example, a subject above the age of 18). In some embodiments, the subject is a child (for example, a subject below the age of 18). In some embodiments, the subject is a male. In some embodiments, the subject is a female. In some embodiments, the subject is affected (e.g., diagnosed), with an autoimmune disease, such as an autoantibody-mediated autoimmune disease. In some embodiments, the subject is at risk of developing an autoimmune disease, such as an autoantibody-mediated autoimmune disease. Examples of risk factors include, but are not limited to, genetic predisposition and familial history of autoantibody-mediated autoimmune disease.

The term “substantially purified cell” refers to a cell that is essentially free of other cell types. A substantially purified cell also refers to a cell that has been separated from other cell types with which it is normally associated in its naturally occurring state. In some embodiments, a substantially purified cell refers to a cell that is at least about 75% free, 80% free, or 85% free, or about 90%, 95%, 96%, 97%, 98%, or 99% free, from other cell types with which it is normally associated in its naturally occurring state. In some instances, a population of substantially purified cells refers to a homogenous population of cells. In some embodiments, a population of substantially purified cells refers to a population of cells at least about 75% homogenous, 80% homogenous, or 85% homogenous, and in particular embodiments about 90%, 95%, 96%, 97%, 98%, or 99% homogenous. In some instances, substantially purified cells simply refer to cells that have been separated from the cells with which they are naturally associated in their natural state. In some aspects, the cells are cultured in vitro. In other aspects, the cells are not cultured in vitro. In certain embodiments, a cell described herein cannot be used to generate a multicellular organism.

The term “T cell” includes all types of immune cells expressing CD3 including CD4⁺ cells (e.g., T helper cells), CD8⁺ T cells (e.g., cytotoxic CD8⁺ T cells and regulatory CD8⁺ T cells), T regulatory cells (Tregs), gamma-delta T cells, and double negative T cells.

The term “therapeutically effective amount” refers to an amount of an agent (e.g., cells expressing a CAR as described herein) effective to achieve a particular biological result. Thus, the term “therapeutically effective amount” means a level or amount of agent that is aimed at, without causing significant negative or adverse side effects to the target, (1) delaying or preventing the onset of the targeted disease or condition; (2) slowing down or stopping the progression, aggravation, or deterioration of one or more symptoms of the targeted disease or condition; (3) bringing about amelioration of the symptoms of the targeted disease or condition; (4) reducing the severity or incidence of the targeted disease or condition; or (5) curing the targeted disease or condition. In some embodiments, a therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.

The term “transfected” or “transformed” or “transduced” refers to a process by which exogenous nucleic acid is transferred or introduced into a host cell. A “transfected” or “transformed” or “transduced” cell is one that has been transfected, transformed or transduced with exogenous nucleic acid. The cell includes the primary subject cell and its progeny.

The term “transfer vector” refers to a composition of matter that comprises an isolated nucleic acid and that can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to include non-plasmid and non-viral compounds that facilitate transfer of nucleic acid into cells, such as, for example, a poly lysine compound, liposome, and the like. Examples of viral transfer vectors include, but are not limited to, adenoviral vectors, adeno-associated virus vectors, retroviral vectors, lentiviral vectors, and the like.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

A “transplant” as used herein, refers to cells, tissue, or an organ that are introduced into an individual. The source of the transplanted material can be cultured cells, cells from another individual, or cells from the same individual (e.g., after the cells are cultured in vitro and optionally altered). Exemplary organ transplants are kidney, liver, heart, lung, and pancreas. An exemplary tissue transplant is islets. An exemplary cell transplant is an allogeneic hematopoietic stem cell transplant.

As used herein, the terms “treat,” “treatment” and “treating” refer to the reduction or amelioration of the progression, severity and/or duration of a targeted disease or condition, e.g., an autoimmune condition, or the amelioration of one or more symptoms (e.g., one or more discernible symptoms) of a targeted disease or condition, e.g., an autoimmune condition, wherein said amelioration results from the administration of one or more therapies (e.g., one or more therapeutic agents such as a Treg cell of the invention). In specific embodiments, the terms “treat,” “treatment” and “treating” refer to the amelioration of at least one measurable physical parameter of a targeted disease or condition, e.g., an autoimmune condition. In some embodiments, the terms “treat,” “treatment” and “treating” refer to inhibition of the progression of a targeted disease or condition, e.g., an autoimmune condition, either physically by, e.g., stabilization of a discernible symptom, physiologically by, e.g., stabilization of a physical parameter, or both. In some embodiments, the terms “treat,” “treatment” and “treating” refer to the reduction or amelioration of the progression, severity and/or duration of a targeted disease or condition, e.g., an autoimmune disease, or the amelioration of one or more symptoms of a targeted disease or condition, e.g., an autoimmune disease. “Treating” or “treatment” refers to both therapeutic treatment and prophylactic or preventative measures; wherein the object is to prevent or slow down (lessen) the targeted disease or condition. Those in need of treatment include those already with the condition as well as those prone to having the condition or those in whom the condition is to be prevented. A subject is successfully “treated” for a disease or condition if, after receiving a therapeutic amount of an agent (e.g., a population of cells comprising a chimeric receptor according to the present invention), the subject shows observable and/or measurable improvement in one or more of the following: reduction in the number of pathogenic cells; reduction in the percent of total cells that are pathogenic; relief to some extent of one or more of the symptoms associated with the specific condition; reduced morbidity and mortality, and/or improvement in quality of life issues. The above parameters for assessing successful treatment and improvement in the condition are readily measurable by routine procedures familiar to a physician.

The term “Treg cell” refers to a cell capable of suppressing, inhibiting or preventing excessive or unwanted inflammatory responses, such as, for example, autoimmunity or allergic reactions. In some embodiments, the Treg cell population of the invention is capable of suppressive activity. In some embodiments, said suppressive activity is contact independent. In some embodiments, said suppressive activity is contact dependent. In some embodiments, the Treg cell population of the invention presents a suppressive action on effector T cells; in certain embodiments, said suppressive action is dependent on TCR expression and/or activation.

A “unibody” is well known in the art and refers to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

The term “variable” refers to the fact that certain segments of the variable (V) domains differ extensively in sequence among antibodies. The V domain mediates antigen binding and defines specificity of a particular antibody for its particular antigen. However, the variability is not evenly distributed across the 110 to 130-amino acid span of a variable domain. Instead, V regions consist of relatively invariant stretches called framework regions (FRs) of 15-30 amino acids separated by shorter regions of extreme variability called “hypervariable regions” or “CDRs” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term polynucleotide “variant” as used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or by using any of a number of techniques well known in the art. Accordingly, the term “polypeptide variant” as used herein, is a polypeptide that typically differs from a polypeptide specifically disclosed herein in one or more substitutions, deletions, additions and/or insertions. Such variants may be naturally occurring or may be synthetically generated, for example, by modifying one or more of the polypeptide sequences of the invention and evaluating one or more biological activities of the polypeptide as described herein and/or by using any of a number of techniques well known in the art. Modifications may be made in the structure of the polynucleotides and polypeptides of the present invention and still result in a functional molecule that encodes or is a variant or derivative polypeptide with desirable characteristics. When it is desired to alter the amino acid sequence of a polypeptide to create an equivalent, or even improved, variant or portion of a polypeptide of the invention, one skilled in the art will typically change one or more of the codons of the encoding DNA sequence. For example, certain amino acids may be substituted for other amino acids in a protein structure without appreciable loss of its function (e.g., ability to bind other polypeptides (e.g., antigens) or cells). Since it is the binding capacity and nature of a protein that define that protein's biological functional activity, certain amino acid sequence substitutions can be made in a protein sequence, and, of course, its underlying DNA coding sequence, and result in a protein with similar properties. It is thus contemplated that various changes may be made in the peptide sequences of the present invention, or corresponding DNA sequences that encode said peptides, without appreciable loss of their biological utility or activity. In many instances, a polypeptide variant will contain one or more conservative substitutions. A variant may also, or alternatively, contain non-conservative changes. In some embodiments, variant polypeptides differ from a native sequence by substitution, deletion or addition of six, five, four, three, two or one amino acid(s). Variants may also (or alternatively) be modified by, for example, the deletion or addition of amino acids that have minimal influence on the immunogenicity, secondary structure and hydropathic nature of the polypeptide.

“Versabodies” are well known in the art and refer to another antibody mimetic technology. They are small proteins of 3-5 kDa with >15% cysteines, which form a high disulfide density scaffold, replacing the hydrophobic core of typical proteins.

The term “xenogeneic” refers to any material derived from an individual of a different species. The term “xenograft” refers to a graft derived from an individual of a different species.

The term “zeta” or alternatively “zeta chain,” “CD3 zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3 zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In some embodiments, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

### Chimeric Antigen Receptors (CARs)

The present invention provides a chimeric receptor comprising:
at least one extracellular binding domain,
optionally at least one extracellular hinge domain,
at least one transmembrane domain (e.g., a human TNFR2 transmembrane domain or a fragment or variant thereof, any transmembrane domain or a fragment or variant thereof, or any combination thereof), and
at least one intracellular domain (comprising at least one primary intracellular signaling domain and optionally comprising at least one costimulatory intracellular signaling domain, wherein the at least one costimulatory intracellular signaling domain is a human TNFR2 costimulatory intracellular signaling domain or a fragment or variant thereof, any costimulatory intracellular signaling domain or a fragment or variant thereof, or any combination thereof), wherein the transmembrane domain is a TNFR2 transmembrane domain or a fragment or variant thereof, and/or the costimulatory intracellular signaling domain is a TNFR2 costimulatory intracellular signaling domain or a fragment or variant thereof.
   In some embodiments, the chimeric receptor comprises one or more polypeptides.

In some embodiments, the extracellular binding domain is an antigen-binding domain, and the chimeric receptor thus may also be referred to as a chimeric antigen receptor (or CAR).

### I. Extracellular Binding Domains

In some embodiments, the extracellular domain comprises an antigen-binding domain, e.g., an antibody or antigen-binding fragment thereof. The portion of the chimeric receptor of the invention comprising an antibody or antigen-binding fragment thereof may exist in a variety of forms where the ligand binding domain is expressed as part of a contiguous polypeptide chain including, for example, a single domain antibody fragment (sdAb), a single chain antibody (scFv), a humanized antibody or a bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., Proc. Natl. Acad. Sci. USA 85:5879-5883 (1988); Bird et al., Science 242:423-426 (1988)). In some aspects, the antigen-binding domain of a chimeric receptor described herein comprises an antibody fragment. In some aspects, the chimeric receptor comprises an antibody fragment that comprises an scFv.

In some embodiments, said antibody is an antibody molecule selected from the group consisting of a whole antibody, a humanized antibody, a single chain antibody, a dimeric single chain antibody, a Fv, an scFv, a Fab, a F(ab)′₂, a defucosylated antibody, a bi-specific antibody, a diabody, a triabody, and a tetrabody.

In some embodiments, said antibody is an antibody fragment selected from the group consisting of a unibody, a single domain antibody, and a nanobody.

In some embodiments, said antibody is an antibody mimetic selected from the group consisting of an affibody, an affilin, an affitin, an adnectin, an atrimer, an evasin, a DARPin, an anticalin, an avimer, a fynomer, a versabody and a duocalin.

DARPins (Designed Ankyrin Repeat Proteins) are well known in the art and refer to an antibody mimetic DRP (designed repeat protein) technology developed to exploit the binding abilities of non-antibody polypeptides.

Fragments and derivatives of antibodies of this invention (which are encompassed by the term “antibody” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules, (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., JMB 273:927-948 (1997) (“Chothia” numbering scheme), or a combination thereof.

In some embodiments, the antigen-binding domain of a CAR of the invention comprises or consists of an antibody fragment, such as, for example, an scFv. In particular embodiments, the antigen-binding domain is an scFv.

In some embodiments, the antigen-binding domain of a CAR of the invention recognizes a specific antigen or fragment thereof (e.g., associated with a target cell). Thus, the antigen-binding domain of a CAR may recognize target cells such as, for example, infected cells, damaged cells, or dysfunctional cells. Examples of such target cells may include cells involved in dysfunctional immune reactions (e.g., cells involved in autoimmune diseases or allergy), dysfunctionally activated inflammatory cells (e.g., inflammatory endothelial cells), cancer cells, and infected (e.g., virally, bacterially, or parasitically infected) cells.

As used herein, the term “fragment” of an antigen refers to any subset of an antigen, as a shorter peptide. In some embodiments, a fragment of an antigen is a peptide of at least 6 amino acids in length. In some embodiments, a fragment of an antigen is a peptide of 6 to 50 amino acids in length, of 6 to 30 amino acids, or of 6 to 20 amino acids in length.

The term “variant” of an antigen refers herein to an antigen that is almost identical to the natural antigen and which shares the same biological activity. The minimal difference between the natural antigen and its variant may lie for example in an amino acid substitution, deletion, and/or addition. Such variants may contain, for example, conservative amino acid substitutions. In some embodiments, the variant of an antigen presents a sequence identity of at least or of about 70, 75, 80, 85, 90, 91, 92, 93, 94, 95, 96, 97, 98, or 99% with the sequence of the natural antigen.

In some embodiments, the antigen is an autoantigen. Examples of autoantigens include, without limitation, an antigen associated with an inflammatory nervous system condition (e.g., a multiple sclerosis-associated antigen), a joint-associated antigen, an eye-associated antigen, a human HSP antigen, a skin-associated antigen or an antigen involved in graft rejection or GVHD.

Examples of multiple sclerosis-associated antigens include, without limitation, myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin-11), heat shock proteins, oligodendrocyte specific proteins (OSP), NOGO A, glycoprotein Po, peripheral myelin protein 22 (PMP22), 2′3′-cyclic nucleotide 3′-phosphodiesterase (CNPase), or any fragments, variants or mixtures thereof.

Examples of joint-associated antigens include, without limitation, citrulline-substituted cyclic and linear filaggrin peptides, type II collagen peptides, citrullinated vimentin, citrullinated type II collagen, citrullinated fibrinogen, human cartilage glycoprotein 39 (HCgp39) peptides, HSP, heterogeneous nuclear ribonucleoprotein (hnRNP) A2 peptides, hnRNP B1, hnRNP D, Ro60/52, HSP60, HSP65, HSP70 and HSP90, BiP, keratin, vimentin, fibrinogen, type I, III, IV and V collagen peptides, annexin V, glucose 6 phosphate isomerase (GPI), acetyl-calpastatin, pyruvate dehydrogenase (PDH), aldolase, topoisomerase I, snRNP, PARP, Scl-70, Scl-100, phospholipid antigens including anionic cardiolipin and phosphatidylserine, neutrally charged phosphatidylethanolamine and phosphatidylcholine, matrix metalloproteinase, fibrillin, aggrecan, and fragments, variants and mixtures thereof.

Examples of eye-associated antigens include, without limitation, type II collagen, citrullinated vimentin, citrullinated type II collagen, citrullinated fibrinogen, retinal arrestin, S-arrestin, interphotoreceptor retinoid-binding proteins (IRBP1), beta-crystallin B1, retinal proteins, choroid proteins and fragments, variants and mixtures thereof.

Examples of human HSP antigens include, without limitation, human HSP60, HSP70, HSP90, and fragments, variants and mixtures thereof.

In some embodiments, the antigen is an inflammatory nervous system condition-associated antigen, e.g., a multiple sclerosis-associated antigen. Examples of inflammatory nervous system condition-associated antigens (e.g., multiple sclerosis-associated antigens) include, but are not limited to, myelin basic protein (MBP), myelin associated glycoprotein (MAG), myelin oligodendrocyte protein (MOG), proteolipid protein (PLP), oligodendrocyte myelin oligoprotein (OMGP), myelin associated oligodendrocyte basic protein (MOBP), oligodendrocyte specific protein (OSP/Claudin-11), heat shock proteins, oligodendrocyte specific proteins (OSP), NOGO A, glycoprotein Po, peripheral myelin protein 22 (PMP22), 2′3′-cyclic nucleotide 3′-phosphodiesterase (CNPase), and fragments, variants and mixtures thereof.

In some embodiments, the antigen is a joint-associated antigen. Examples of joint-associated antigens include, but are not limited to, citrulline-substituted cyclic and linear filaggrin peptides, collagen type II peptides, human cartilage glycoprotein 39 (HCgp39) peptides, HSP, heterogenous nuclear ribonucleoprotein (hnRNP) A2 peptides, hnRNP B1, hnRNP D, Ro60/52, HSP60, 65, 70 and 90, BiP, keratin, vimentin, fibrinogen, collagen type I, III, IV and V peptides, annexin V, glucose 6 phosphate isomerase (GPI), acetyl-calpastatin, pyruvate deshydrogenase (PDH), aldolase, topoisomerase I, snRNP, PARP, Scl-70, Scl-100, phospholipid antigen including anionic cardiolipin and phosphatidylserine, neutrally charged phosphatidylethanolamine and phosphatidylcholine, matrix metalloproteinase, fibrillin, aggreccan, and fragments, variants and mixtures thereof.

In some embodiments, the antigen is an eye-associated antigen. Examples of eye-associated antigens include, but are not limited to, type II collagen, retinal arrestin, S-arrestin, interphotoreceptor retinoid-binding proteins (IRBP1), betaB1-crystallin, retinal proteins, choroid proteins, and fragments, variants and mixtures thereof.

In some embodiments, the antigen is a human HSP antigen. Examples of human HSP antigens include, but are not limited to human HSP60, HSP70, HSP90, and fragments, variants and mixtures thereof.

In some embodiments, the antigen is a skin-associated antigen. Examples of skin-associated antigens include, but are not limited to, keratinocyte antigens, an antigen present in the dermis or epidermis, a melanocyte antigen (such as, for example, melanin or tyrosinase), desmoglein (e.g., desmoglein 1 or 3, which may also be referred to as Dsg1/3), BP180, BP230, plectin, integrins (e.g., integrin α4β6), collagens (e.g., collagen type VII), laminins (e.g., laminin 332 or laminin yl), plakins (e.g., envoplakin, periplakin, or desmoplakins), keratins (e.g., KRT5, KRT8, KRT15, KRT17 and KRT31), keratin filament-associated proteins, filaggrin, corneodesmosin, and elastin.

In some embodiments, the antigen is an antigen involved in graft rejection or GVHD. Examples of such antigens include, but are not limited to, the MHC specific to the transplanted tissue or to the host, β2-microglobulin, antigens from the ABO system, antigens from the rhesus system (e.g., antigens C, c, E, e and D) and isohaemagglutinins. Other examples of antigens that may be involved in graft rejection or GVHD include, but are not limited to, HLA-DR (in particular during the first six months following grafting), HLA-B (in particular during the first two years following grafting), HLA-A, minor histocompatibility antigens (miHA, e.g., HLA-E, HLA-F and HLA-G), HLAs corresponding to MHC class I (A, B, and C), HLAs corresponding to MHC class II (DP, DM, DOA, DOB, DQ, and DR) and HLAs corresponding to MHC class III (e.g., components of the complement system).

In some embodiments, the antigen is an HLA-A2 cell surface protein. In some embodiments, the extracellular binding domain comprises an antibody directed to HLA-A2 or an antigen binding fragment thereof. In some embodiments, the HLA-A2 binding domain comprises an scFv directed to HLA-A2.

The term “HLA-A2” as used herein refers to human leukocyte antigen (HLA) proteins including cell surface proteins, encoded by the HLA-A*02 allele family at the HLA-A locus of the HLA gene complex. HLA proteins encompassed by the term “HLA-A2” include HLA proteins identified as belonging to the HLA-A*02 antigen type by serological testing or genotyping. Additional names for the HLA-A*02 antigen type include “HLA-A2,” HLA-A02” and “HLA-A*2.” Different naming systems have been developed that identify HLA proteins encoded by this family of alleles including the HLA naming system developed in 2010 by the WHO Committee for Factors of the HLA System. The term “HLA-A2” refer to HLA proteins encoded by alleles having designations according to this naming system that begin with “HLA-A*02”, including but not limited to designations that begin with “HLA-A*02:01”, “HLA-A*02:02”, “HLA-A*02:03”, “HLA-A*02:04”, “HLA-A*02:05”, “HLA-A*02:06”, “HLA-A*02:07”, “HLA-A*02:08”, “HLA-A*02:09”, “HLA-A*02:10”, and “HLA-A*02:11”. The allele designations may be italicized. The allele designations that begin with “HLA-A*02:” followed by 2 or 3 additional digits may constitute the complete designation or a beginning portion of the designation. The term “HLA-A2” also refers to HLA proteins identified with designations that begin with “HLA-A*02” according to this naming system, including but not limited to the designations “HLA-A*02:01”, “HLA-A*02:02” “HLA-A*02:03”, “HLA-A*02:04”, “HLA-A*02:05”, “HLA-A*02:06”, “HLA-A*02:07” “HLA-A*02:08”, “HLA-A*02:09”, “HLA-A*02:10”, and “HLA-A*02:11”.

In some embodiments, the HLA-A2 binding domain comprises an antibody directed to HLA-A2 or an antigen binding fragment thereof. In certain embodiments, the HLA-A2 binding domain comprises an scFv directed to HLA-A2. Examples of scFvs directed to HLA-A2 include, but are not limited to, scFvs consisting or comprising of a sequence selected from the group consisting of SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, and fragments or variants thereof; and anti-HLA-A2 scFvs disclosed in PCT Patent Publications WO 2018/183293 and WO 2019/056099. For example, an anti-HLA-A2 scFv used in a CAR described herein may consist of or comprise the amino acid sequence of SEQ ID NO: 68. In yet another example, an anti-HLA-A2 scFv used in a CAR described herein may consist of or comprise the amino acid sequence of SEQ ID NO: 70. In yet another example, an anti-HLA-A2 scFv used in a CAR described herein may consist of or comprise the amino acid sequence of SEQ ID NO: 92. In yet another example, an anti-HLA-A2 scFv used in a CAR described herein may consist of or comprise the amino acid sequence of SEQ ID NO: 103. In yet another example, an anti-HLA-A2 scFv used in a CAR described herein may consist of or comprise the amino acid sequence of SEQ ID NO: 107.

Other examples of autoantigens include, without limitation, aquaporin water channels (such as, for example, aquaporin-4 water channel (AQP4)), Hu, Ma2, collapsin response-mediator protein 5 (CRMP5), amphiphysin, voltage-gated potassium channel (VGKC), N-methyl-d-aspartate receptor (NMDAR), α-amino-3-hydroxy-5-methyl-4-isoxazoleproprionic acid (AMPAR), thyroid peroxidase, thyroglobulin, anti-N-methyl-D-aspartate receptor (NR1 subunit), Rh blood group antigens, I antigen, desmoglein 1 or 3 (Dsg1/3), BP180, BP230, acetylcholine nicotinic postsynaptic receptors, thyrotropin receptors, platelet integrin, GpIIb:IIIa, collagen (such as, for example, collagen alpha-3(IV) chain), rheumatoid factor, calpastatin, citrullinated proteins, myelin basic protein (MBP), myelin oligodendrocyte glycoprotein (MOG) peptides, alpha-beta-crystallin, DNA, histones, ribosomes, RNP, tissue transglutaminase (TG2), intrinsic factor, 65-kDa antigen, phosphatidylserine, ribosomal phosphoproteins, anti-neutrophil cytoplasmic antibody, Scl-70, U1-RNP, ANA, SSA, anti-SSB, antinuclear antibodies (ANA), antineutrophil cytoplasm antibodies (ANCA), Jo-1, antimitochondrial antibodies, gp210, p62, sp100, antiphospholipid antibodies, U1-70 kd snRNP, GQ1b ganglioside, GM1, asialo GM1, GD1b, anti-smooth muscle antibodies (ASMA), anti-liver-kidney microsome-1 antibodies (ALKM-1), anti-liver cytosol antibody-1 (ALC-1), IgA antiendomysial antibodies, neutrophil granule proteins, streptococcal cell wall antigen, intrinsic factor of gastric parietal cells, insulin (IAA), glutamic acid decarboxylase (GAA or GAD), protein tyrosine phosphatase (such as, for example, IA2 or ICA512), PLA2R1 and THSD7A1.

In some embodiments, the antigen is an IL-23 receptor (IL-23R) expressed on the cell surface. In some embodiments, the extracellular binding domain is an antibody directed to IL-23R or an antigen binding fragment thereof.

In some embodiments, the antigen is soluble IL-23R. In some embodiments, the extracellular binding domain is an antibody directed to soluble IL-23R or an antigen binding fragment thereof.

In some embodiments, the antigen is a variant of IL-23R. In some embodiments, the extracellular binding domain is an antibody directed to a variant of IL-23R or an antigen binding fragment thereof.

In some embodiments, a variant peptide of IL-23R is a modified IL-23R peptide wherein 1, 2, 3, 4, 5, 6, 7, 8, 9 or 10 amino acids are deleted, added or substituted as compared to the original peptide.

In some embodiments, the antigen is a splice variant of IL-23R. In some embodiments, the extracellular binding domain is an antibody directed to a splice variant of IL-23R or an antigen binding fragment thereof.

In some embodiments, a CAR of the invention recognizes and is capable of binding to a human IL-23R. In some embodiments, a CAR of the invention recognizes and is capable of binding to a murine IL-23R.

In some embodiments, the IL-23R binding domain comprises an antibody directed to IL-23R or an antigen binding fragment thereof. In some embodiments, the IL-23R binding domain comprises an scFv directed to IL-23R. Examples of scFvs directed to IL-23R include, but are not limited to, scFvs consisting or comprising of a sequence selected from the group consisting of SEQ ID NO: 65, SEQ ID NO: 66, SEQ ID NO: 67, and fragments or variants thereof.

In some embodiments, the antigen is an inhaled allergen, an ingested allergen or a contact allergen.

In some embodiments, the antigen is a food antigen from common human diet.

The term “food antigen from common human diet” refers to an immunogenic peptide that comes from foodstuffs common for humans, such as a food antigen of the following non-limiting list: ovalbumin, bovine antigens such as lipocalin, Ca-binding S100, alpha-lactalbumin, lactoglobulins such as beta-lactoglobulin, bovine serum albumin, and caseins. Food antigens may also be Atlantic salmon antigens such as parvalbumin, chicken antigens such as ovomucoid, Ag22, conalbumin, lysozyme or chicken serum albumin, peanuts, shrimp antigens such as tropomyosin, wheat antigens such as agglutinin or gliadin, celery antigens such as celery profilin, carrot antigens such as carrot profilin, apple antigens such as thaumatin, apple lipid transfer protein, apple profilin, pear antigens such as pear profilin, isoflavone reductase, avocado antigens such as endochitinase, apricot antigens such as apricot lipid transfer protein, peach antigens such as peach lipid transfer protein or peach profilin, soybean antigens such as HPS, soybean profilin or (SAM22) PR-I0 prot, and fragments, variants and mixtures thereof.

In some embodiments, the antigen is a tissue-specific protein. Examples of tissue-specific proteins include, but are not limited to, integrins and selectins whose expression is limited to a specific tissue or organ.

In some embodiments, the antigen is a B cell surface marker expressed at the surface of a B cell. Examples of surface markers of B cells (e.g., human B cells) include, but are not limited to, CD19, CD20, BCMA, IgM, IgA, IgG, IgE, IgD, CD1, CD5, CD21, CD22, CD23, CD24, CD25, CD27, CD30, CD38, CD40, CD78, CD80, CD138, CD319, PDL-2, CXCR3, CXCR4, CXCR5, CXCR6, Notch2, TLR4, IL-6, IL-10 and TGFβ. In certain embodiments, the B cell surface marker is selected from CD19, CD20, BCMA, IgM, IgA, IgG, IgE, IgD, CD1, CD21, CD22, CD138. In particular embodiments, the B cell surface marker is selected from CD19 and CD20.

In some embodiments, the antigen is a proB cell surface marker expressed at the surface of a proB cell. Examples of surface markers of proB cells (e.g., human proB cells) include, but are not limited to, CD10, CD19, CD24, CD34 and CD38.

In some embodiments, the antigen is a preB cell surface marker expressed at the surface of a preB cell. Examples of surface markers of preB cells (e.g., human preB cells) include, but are not limited to, CD5, CD10, CD19, CD20 and CD34, CD38.

In some embodiments, the antigen is an immature (or transitional) B cell surface marker expressed at the surface of a B cell. Examples of surface markers of immature (or transitional) B cells (e.g., human immature B cells) include, but are not limited to, CD5, CD10, CD19, CD20, CD22, CD24, CD38 and IgG.

In some embodiments, the antigen is a marginal zone B cell surface marker expressed at the surface of a B cell. Examples of surface markers of marginal zone B cells (e.g., human marginal zone B cells) include, but are not limited to, CD1, CD19, CD20, CD21, CD22, CD23, CD27, IgG and Notch2.

In some embodiments, the antigen is a plasma cell surface marker expressed at the surface of a B cell. Examples of surface markers of plasma cells (e.g., human plasma cells) include, but are not limited to, CD19, CD27, CD38, CD138, IgG, MHCII, and IL-6.

In some embodiments, the antigen is a plasmablast cells surface marker expressed at the surface of a B cell. Examples of surface markers of plasmablasts (e.g., human plasmablasts) include, but are not limited to, CD19, CD20, CD27, CD38, IgG, and MHCII.

In some embodiments, the antigen is a memory B cell surface marker expressed at the surface of a B cell. Examples of surface markers of memory B cells (e.g., human memory B cells) include, but are not limited to, CD19, CD20, CD22, CD24, CD27, CD38, CD40, CD80, PD-L2, IgG, CXCR3, CXCR4, CXCR5, CXCR6, IgA, IgG and IgE.

In some embodiments, the antigen is a germinal center B cell surface marker expressed at the surface of a B cell. Examples of surface markers of germinal center B cells (e.g., human germinal center B cells) include, but are not limited to, CD10, CD19, CD20, CD22, CD38, and IgG.

In some embodiments, the antigen is an activated B cell surface marker expressed at the surface of a B cell. Examples of surface markers of activated B cells (e.g., human activated B cells) include, but are not limited to, CD19, CD25, and CD30.

In some embodiments, the antigen is a regulatory B cell surface marker expressed at the surface of a B cell. Examples of surface markers of regulatory B cells (Breg cells) include, but are not limited to, CD1, CD1d, CD5, CD19, CD21, CD23, CD24, CD40, Fas ligand, IL-10, TLR4, TGFβ, IgD, IgM, PD-L1, PD-L2, TIM-1, TNFSF18, and TRAIL. In particular embodiments, examples of surface markers of human Breg cells include, but are not limited to, CD1, CD1d, CD5, CD19, CD21, CD24, CD40, IL-10, TLR4, TGFβ, IgD and IgM.

In some embodiments, the antigen is a B cell surface marker with non-secreted Ig expressed at the surface of a B cell. Examples of surface markers of B cells (e.g., human B cells) with non-secreted Ig include, but are not limited to, CD138 and Notch2.

In some embodiments, the B cell surface marker is CD19. In some embodiments, the CD19 binding domain comprises an antibody directed to CD19 or an antigen binding fragment thereof. In some embodiments, the CD19 binding domain comprises an scFv directed to CD19. Examples of scFvs directed to CD19 include, but are not limited to, SEQ ID NO: 1 and FMC63 (SEQ ID NO: 2).

In some embodiments, the B cell surface marker is CD20. In some embodiments, the CD20 binding domain comprises an antibody directed to CD20 or an antigen binding fragment thereof. Examples of CD20 antibodies include, but are not limited to, rituximab or a fragment or variant thereof (e.g., SEQ ID NO: 3 or a fragment or variant thereof). Examples of scFvs directed to CD20 include, but are not limited to, scFvs comprising or consisting of a sequence selected from the group consisting of SEQ ID NO: 4, SEQ ID NO: 5, SEQ ID NO: 6, and fragments or variants thereof.

In some embodiments, the antigen is a cancer antigen.

As used herein, the term “cancer antigen” refers to an antigen that is differentially expressed by cancer cells and can therefore be exploited to target cancer cells. Cancer antigens are antigens that can potentially stimulate apparently tumor-specific immune responses. Some of these antigens are encoded, although not necessarily expressed, by normal cells; these antigens can be characterized as those that are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3 (defined by immunity), MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

Other cancer antigens include, but are not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (lnterleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), L1 CAM (L1 cell adhesion molecule), MAGE (melanoma antigen), MAGE-A1 (melanoma-associated antigen 1), mesothelin, murine CMV infected cells, MART-1/Melan-A (melanoma antigen recognized by T cells-I/melanoma antigen A), MC1 R (melanocortin 1 receptor), yosin/m (myosin mutated), MUC1 (mucin 1), MUM-1, -2, -3 (melanoma ubiquitous mutated-1, -2, -3), NA88-A (NA cDNA clone of patient M88), NKG2D (natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), synovial sarcoma X-1, -2, -3, -4 (SSX-1, -2, -3, -4), TAA (tumor-associated antigen), TAG-72 (tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene).

In some embodiments, the antigen is associated with infected cells.

As used herein, the term “infected cells” refers to cells contaminated with something that affects their quality, character, or condition unfavorably.

In some embodiments, the antigen is associated with virally infected cells. In some embodiments, the antigen is associated with bacterially infected cells. In some embodiments, the antigen is associated with fungally infected cells. In some embodiments, the antigen is associated with parasite infected cells.

In some embodiments, the extracellular binding domain is a protein or a fragment or a variant thereof.

In some embodiments, the extracellular binding domain recognizes an autoantibody on a B cell.

In some embodiments, the extracellular binding domain is an autoantigen.

In some embodiments, the chimeric receptor comprises an autoantigen (that may also be referred as self-antigen) or a fragment or variant thereof, and thus can recognize antibodies directed to said autoantigen. As used herein, the term “autoantigen” or “self-antigen” refers to an endogenous antigen that stimulates production of autoantibodies. In some embodiments, the autoantigen is involved in an autoimmune disease. In some embodiments, the immune cells of the invention are cytotoxic for B cells producing antibodies directed to said autoantigen.

The term “variant of an autoantigen” refers herein to an autoantigen that is almost identical to the natural autoantigen and that shares the same biological activity. The minimal difference between the natural autoantigen and a variant thereof may lie, for example in an amino acid substitution, deletion, and/or addition. Such variants may contain, for example, conservative amino acid substitutions in which amino acid residues are replaced with amino acid residues having a similar side chain. Families of amino acid residues having similar side chains have been defined in the art, including basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine). In some embodiments, the variant of an autoantigen presents a sequence identity of at least 70, 75, 80, 85, 90, 91, 92, 93, 94, 95, 96, 97, 98, or 99% with the sequence of the natural autoantigen.

Examples of autoantigens include, but are not limited to, aquaporin water channels (such as, for example, aquaporin-4 water channel (AQP4)), Hu, Ma2, collapsin response-mediator protein 5 (CRMP5), amphiphysin, voltage-gated potassium channel (VGKC), N-methyl-d-aspartate receptor (NMDAR), α-amino-3-hydroxy-5-methyl-4-isoxazoleproprionic acid (AMPAR), thyroid peroxidase, thyroglobulin, anti-N-methyl-D-aspartate receptor (NR1 subunit), Rh blood group antigens, I antigen, desmoglein 1 or 3 (Dsg1/3), BP180, BP230, acetylcholine nicotinic postsynaptic receptors, thyrotropin receptors, platelet integrin, GpIIb:IIIa, collagen (such as, for example, collagen alpha-3(IV) chain), rheumatoid factor, calpastatin, citrullinated proteins, myelin basic protein (MBP), myelin oligodendrocyte glycoprotein (MOG) peptides, alpha-beta-crystallin, DNA, histone, ribosomes, RNP, tissue transglutaminase (TG2), intrinsic factor, 65-kDa antigen, phosphatidylserine, ribosomal phosphoproteins, anti-neutrophil cytoplasmic antibody, Scl-70, U1-RNP, ANA, SSA, anti-SSB, antinuclear antibodies (ANA), antineutrophil cytoplasm antibodies (ANCA), Jo-1, antimitochondrial antibodies, gp210, p62, sp100, antiphospholipid antibodies, U1-70 kd snRNP, GQ1b ganglioside, GM1, asialo GM1, GD1b, anti-smooth muscle antibodies (ASMA), anti-liver-kidney microsome-1 antibodies (ALKM-1), anti-liver cytosol antibody-1 (ALC-1), IgA antiendomysial antibodies, neutrophil granule proteins, streptococcal cell wall antigen, intrinsic factor of gastric parietal cells, insulin (IAA), glutamic acid decarboxylase (GAA or GAD), protein tyrosine phosphatase (such as, for example, IA2 or ICA512), PLA2R1 and THSD7A1.

Other examples of autoantigens are listed herein and include, without limitation, multiple sclerosis-associated antigens (such as, for example, myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin-11), heat shock proteins, oligodendrocyte specific proteins (OSP), NOGO A, glycoprotein Po, peripheral myelin protein 22 (PMP22), 2′3′-cyclic nucleotide 3′-phosphodiesterase (CNPase), and fragments, variants and mixtures thereof); joint-associated antigens (such as, for example, citrulline-substituted cyclic and linear filaggrin peptides, type II collagen peptides, human cartilage glycoprotein 39 (HCgp39) peptides, HSP, heterogeneous nuclear ribonucleoprotein (hnRNP) A2 peptides, hnRNP B1, hnRNP D, Ro60/52, HSP60, HSP65, HSP70 and HSP90, BiP, keratin, vimentin, fibrinogen, type I, III, IV and V collagen peptides, annexin V, glucose 6 phosphate isomerase (GPI), acetyl-calpastatin, pyruvate dehydrogenase (PDH), aldolase, topoisomerase I, snRNP, PARP, Scl-70, Scl-100, phospholipid antigens including anionic cardiolipin and phosphatidylserine, neutrally charged phosphatidylethanolamine and phosphatidylcholine, matrix metalloproteinase, fibrillin, aggreccan, citrullinated vimentin, citrullinated type II collagen, citrullinated fibrinogen, and fragments, variants and mixtures thereof); eye-associated antigens (such as, for example, type II collagen, retinal arrestin, S-arrestin, interphotoreceptor retinoid-binding proteins (IRBP1), beta-crystallin B1, retinal proteins, choroid proteins, citrullinated vimentin, citrullinated type II collagen, citrullinated fibrinogen and fragments, and variants and mixtures thereof); and human HSP antigens (such as, for example, human HSP60, HSP70, HSP90, and fragments, variants and mixtures thereof).

In some embodiments, the autoantigen is desmoglein 1 or desmoglein 3 or a variant or fragment thereof, such as, for example, extracellular domains of desmoglein 1 or 3. In some embodiments, the chimeric receptor comprises extracellular domains 1 to 4 of desmoglein 3, such as, for example, a sequence comprising or consisting of SEQ ID NO: 7.

In some embodiments, a CAR of the invention comprises an extracellular binding domain against a first antigen and at least one other extracellular binding domain against another antigen. Such a CAR is capable of binding to at least 2 different antigens. In certain embodiments, said at least one other extracellular binding domain is an antibody directed to a specific antigen or an antigen binding fragment thereof. In certain embodiments, said at least one other extracellular binding domain comprises or consists of an antibody fragment, such as, for example, an scFv.

In some embodiments, the antibody comprised in a CAR of the invention is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, the multispecific antibody molecule is a bispecific antibody molecule. A bispecific antibody has specificity for two antigens, and is characterized by a first immunoglobulin variable domain sequence that has binding specificity for a first epitope and a second immunoglobulin variable domain sequence that has binding specificity for a second epitope.

### II. Spacer and Hinge Domains

In some embodiments, the extracellular binding domain is connected to a transmembrane domain by a spacer domain or a hinge domain. Examples of linkers include, but are not limited to, GS linkers as described herein. In certain embodiments, the linker may comprise or consist of the sequence GGGGSGGGGSGGGGS (SEQ ID NO: 111).

In some embodiments, a short oligo- or polypeptide linker, having a length ranging from, e.g., 2 and 10 amino acids, may form the hinge domain. In some embodiments, the term “linker” refers to a flexible polypeptide linker.

For example, a glycine-serine doublet may provide a suitable hinge domain (GS linker). In some embodiments, the hinge domain is a Gly/Ser linker. Examples of Gly/Ser linkers include, but are not limited to, GS linkers, G₂S linkers, G₃S linkers, and G₄S linkers.

Examples of G₂S linkers include, but are not limited to, GGS.

G₃S linkers comprise the amino acid sequence (Gly-Gly-Gly-Ser)ₙ, also referred to as (GGGS)ₙ or (SEQ ID NO: 112)ₙ, where n is a positive integer equal to or greater than 1 (such as, for example, n=1, n=2, n=3. n=4, n=5, n=6, n=7, n=8, n=9 or n=10). Examples of G₃S linkers include, but are not limited to, GGGSGGGSGGGSGGGS (SEQ ID NO: 113).

Examples of G₄S linkers include, but are not limited to, (Gly₄ Ser) corresponding to GGGGS (SEQ ID NO: 114); (Gly₄ Ser)₂ corresponding to GGGGSGGGGS (SEQ ID NO: 115); (Gly₄Ser)₃ corresponding to GGGGSGGGGSGGGGS (SEQ ID NO: 116); and (Gly₄Ser)₄ corresponding to GGGGSGGGGSGGGGSGGGGS (SEQ ID NO: 117).

In some embodiments, a spacer domain may have a length of up to 300 amino acids, e.g., 10-100 amino acids, 25-50 amino acids, or 2-10 amino acids.

In some embodiments, the hinge domain is a short oligo- or polypeptide linker, e.g., having a length ranging from 2 to 10 amino acids, as described herein. An example of a hinge domain that may be used in the present invention is described in PCT Patent Publication WO2012/138475, incorporated herein by reference.

In some embodiments, the hinge domain comprises an amino acid sequence selected from the group consisting of the amino acid sequence AGSSSSGGSTTGGSTT (SEQ ID NO: 8), the amino acid sequence GTTAASGSSGGSSSGA (SEQ ID NO: 9), the amino acid sequence SSATATAGTGSSTGST (SEQ ID NO: 10), and the amino acid sequence

(SEQ ID NO: 11)
TSGSTGTAASSTSTST.

In some embodiments, the hinge domain is encoded by a nucleotide sequence of

(SEQ ID NO: 12)
GGTGGCGGAGGTTCTGGAGGTGGAGGTTCC.

In some embodiments, the hinge domain is a KIR₂DS₂ hinge corresponding to KIRRDSS (SEQ ID NO: 13).

In some embodiments, the hinge domain comprises or consists of the amino acid sequence of a CD8 hinge (e.g., comprising or consisting of the amino acid sequence of SEQ ID NO: 14) or an amino acid sequence with at least about 70%, 75%, 80%, 85%, 90%, 91%, 92%, 93%, 94%, 95%, 96%, 97%, 98% or 99% identity to SEQ ID NO: 14. In some embodiments, the hinge domain is a CD8 hinge encoded by the nucleic acid sequence of SEQ ID NO: 15 or a nucleic acid sequence with at least about 70%, 75%, 80%, 85%, 90%, 91%, 92%, 93%, 94%, 95%, 96%, 97%, 98% or 99% identity to SEQ ID NO: 15.

In some embodiments, the hinge domain comprises or consists of the amino acid sequence of a IgG4 hinge (e.g., comprising or consisting of the amino acid sequence of SEQ ID NO: 16), or an amino acid sequence with at least about 70%, 75%, 80%, 85%, 90%, 91%, 92%, 93%, 94%, 95%, 96%, 97%, 98% or 99% identity to SEQ ID NO: 16. In some embodiments, the hinge domain is an IgG4 hinge encoded by the nucleic acid sequence of SEQ ID NO: 17 or a nucleic acid sequence with at least about 70%, 75%, 80%, 85%, 90%, 91%, 92%, 93%, 94%, 95%, 96%, 97%, 98% or 99% identity to SEQ ID NO: 17.

In some embodiments, the hinge domain comprises or consists of the amino acid sequence of an IgD hinge (e.g., comprising or consisting of the amino acid sequence of SEQ ID NO: 18) or an amino acid sequence with at least about 70%, 75%, 80%, 85%, 90%, 91%, 92%, 93%, 94%, 95%, 96%, 97%, 98% or 99% identity to SEQ ID NO: 18. In some embodiments, the hinge domain is an IgD hinge encoded by the nucleic acid sequence of SEQ ID NO: 19 or a nucleic acid sequence with at least about 70%, 75%, 80%, 85%, 90%, 91%, 92%, 93%, 94%, 95%, 96%, 97%, 98% or 99% identity to SEQ ID NO: 19.

In some embodiments, the hinge region comprises or consists of the amino acid sequence of a CD28 hinge (e.g., comprising or consisting of the amino acid sequence of SEQ ID NO: 20) or an amino acid sequence with at least about 70%, 75%, 80%, 85%, 90%, 91%, 92%, 93%, 94%, 95%, 96%, 97%, 98% or 99%70%, 75%, 80%, 85%, 90%, 95%, 96%, 97%, 98% or 99% identity to SEQ ID NO: 20. In some embodiments, the hinge domain is a CD28 hinge encoded by the nucleic acid of SEQ ID NO: 21 or a nucleic acid sequence with at least about 70%, 75%, 80%, 85%, 90%, 91%, 92%, 93%, 94%, 95%, 96%, 97%, 98% or 99% identity to SEQ ID NO: 21.

### III. Transmembrane Domains

Examples of transmembrane domains that may be used in a chimeric receptor of the invention include, but are not limited to, transmembrane domains of TNFR2, CD28, CD8, or of an alpha, beta or zeta chain of a T cell receptor, or of CD3 gamma, CD3 delta, CD3 epsilon, CD3 zeta, CD45, CD4, CD5, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, PD1, ITGAX, CDl1c, ITGB1, CD29, ITGB2, CD18, LFA-1, ITGB7, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the transmembrane domain may comprise the entire transmembrane domain of the molecule from which it is derived, or it may comprise a functional fragment or variant thereof.

In some embodiments, the chimeric receptor comprises at least one transmembrane domain selected from a transmembrane domain of TNFR2, a transmembrane domain of CD8 and a transmembrane domain of CD28.

In some embodiments, the transmembrane domain comprises or consists of the amino acid sequence of a TNFR2 transmembrane domain (e.g., comprising or consisting of the amino acid sequence of SEQ ID NO: 22), or an amino acid sequence with at least about 70%, 75%, 80%, 85%, 90%, 91%, 92%, 93%, 94%, 95%, 96%, 97%, 98% or 99% identity to SEQ ID NO: 22. In some embodiments, the transmembrane domain comprises or consists of an amino acid sequence having at least one, two or three modifications but not more than 20, 10 or 5 modifications compared to the amino acid sequence of SEQ ID NO: 22, or an amino acid sequence with at least about 70%, 75%, 80%, 85%, 90%, 91%, 92%, 93%, 94%, 95%, 96%, 97%, 98% or 99% identity to SEQ ID NO: 22.

In some embodiments, the TNFR2 transmembrane domain is encoded by the nucleotide sequence of SEQ ID NO: 23, or a nucleotide sequence with at least about 70%, 75%, 80%, 85%, 90%, 91%, 92%, 93%, 94%, 95%, 96%, 97%, 98% or 99% identity to SEQ ID NO: 23.

In some embodiments, the TNFR2 transmembrane domain comprises at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the sequence of SEQ ID NO: 22 or from a sequence having at least about 70%, 75%, 80%, 85%, 90%, 91%, 92%, 93%, 94%, 95%, 96%, 97%, 98% or 99% identity to SEQ ID NO:22, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 contiguous amino acids from the sequence of SEQ ID NO: 22. In certain embodiments, the TNFR2 transmembrane domain comprises an amino acid sequence selected from the group consisting of CVIMTQV (SEQ ID NO: 62), VNCVIMTQV (SEQ ID NO: 63), or TALGLLIIGVVNCVIMTQV (SEQ ID NO: 64). In particular embodiments, the TNFR2 transmembrane domain comprises the amino acid sequence of VNCVIMTQV (SEQ ID NO: 63).

In some embodiments, the TNFR2 transmembrane domain is encoded by a nucleotide sequence of at least 6, 9, 12, 15, 18, 21, 24, 27, 30, 33, 36, 39, 42, 45, 48, 51, 54, 57, 60, 63, 66, 69, 72, 75, 78, 81, 84 or 87 nucleotides from the sequence of SEQ ID NO: 23 or from a sequence having at least about 70%, 75%, 80%, 85%, 90%, 91%, 92%, 93%, 94%, 95%, 96%, 97%, 98% or 99% identity to SEQ ID NO: 23, e.g., at least 6, 9, 12, 15, 18, 21, 24, 27, 30, 33, 36, 39, 42, 45, 48, 51, 54, 57, 60, 63, 66, 69, 72, 75, 78, 81, 84 or 87 contiguous nucleotides from the sequence of SEQ ID NO: 23.

In some embodiments, the transmembrane domain comprises or consists of the amino acid sequence of a CD8 transmembrane domain (e.g., comprising or consisting of the amino acid sequence of SEQ ID NO: 24), or an amino acid sequence with at least about 70%, 75%, 80%, 85%, 90%, 91%, 92%, 93%, 94%, 95%, 96%, 97%, 98% or 99% identity to SEQ ID NO: 24. In some embodiments, the transmembrane domain comprises or consists of an amino acid sequence having at least one, two or three modifications but not more than 20, 10 or 5 modifications compared to the amino acid sequence of SEQ ID NO: 24, or an amino acid sequence with at least about 70%, 75%, 80%, 85%, 90%, 91%, 92%, 93%, 94%, 95%, 96%, 97%, 98% or 99% identity to SEQ ID NO: 24.

In some embodiments, the CD8 transmembrane domain is encoded by the nucleotide sequence of SEQ ID NO: 25, or a nucleotide sequence with at least about 70%, 75%, 80%, 85%, 90%, 91%, 92%, 93%, 94%, 95%, 96%, 97%, 98% or 99% identity to SEQ ID NO: 25.

In some embodiments, the CD8 transmembrane domain comprises at least 2, 3, 4, 5, 6, 7, 8, 9, 10, 11, 12, 13, 14, 15, 16, 17, 18, 19, 20, 21, 22, 23 or 24 amino acids from the sequence SEQ ID NO: 24 or from a sequence having at least about 70%, 75%, 80%, 85%, 90%, 91%, 92%, 93%, 94%, 95%, 96%, 97%, 98% or 99% identity with SEQ ID NO: 24, e.g., at least 2, 3, 4, 5, 6, 7, 8, 9, 10, 11, 12, 13, 14, 15, 16, 17, 18, 19, 20, 21, 22, 23 or 24 contiguous amino acids from the sequence of SEQ ID NO: 24.

In some embodiments, the CD8 transmembrane domain is encoded by a nucleotide sequence of at least 6, 9, 12, 15, 18, 21, 24, 27, 30, 33, 36, 39, 42, 45, 48, 51, 54, 57, 60, 63, 66, 69 or 72 nucleotides from the sequence of SEQ ID NO: 25 or from a sequence having at least about 70%, 75%, 80%, 85%, 90%, 91%, 92%, 93%, 94%, 95%, 96%, 97%, 98% or 99% identity with SEQ ID NO: 25, e.g., at least 6, 9, 12, 15, 18, 21, 24, 27, 30, 33, 36, 39, 42, 45, 48, 51, 54, 57, 60, 63, 66, 69 or 72 contiguous nucleotides from the sequence of SEQ ID NO: 25.

In some embodiments, the transmembrane domain comprises or consists of the amino acid sequence of a CD28 transmembrane domain (e.g., comprising or consisting of the amino acid sequence of SEQ ID NO: 26) or an amino acid sequence with at least about 70%, 75%, 80%, 85%, 90%, 91%, 92%, 93%, 94%, 95%, 96%, 97%, 98% or 99% identity to SEQ ID NO: 26. In some embodiments, the transmembrane domain comprises or consists of an amino acid sequence having at least one, two or three modifications but not more than 20, 10 or 5 modifications compared to an amino acid sequence of SEQ ID NO: 26, or an amino acid sequence with at least 70%, 75%, 80%, 85%, 90%, 91%, 92%, 93%, 94%, 95%, 96%, 97%, 98% or 99% identity to SEQ ID NO: 26.

In some embodiments, the transmembrane domain is a CD28 transmembrane domain encoded by the nucleic acid sequence of SEQ ID NO: 27 or a nucleic acid sequence with at least about 70%, 75%, 80%, 85%, 90%, 91%, 92%, 93%, 94%, 95%, 96%, 97%, 98% or 99% identity to SEQ ID NO: 27. In some embodiments, the CD28 transmembrane domain comprises at least 2, 3, 4, 5, 6, 7, 8, 10, 11, 12, 13, 14, 15, 16, 17, 18, 19, 20, 21, 22, 23, 24, 25, 26 or 27 amino acids from the sequence of SEQ ID NO: 26 or from a sequence having at least about 70%, 75%, 80%, 85%, 90%, 91%, 92%, 93%, 94%, 95%, 96%, 97%, 98% or 99% identity with SEQ ID NO: 26, e.g., at least, 3, 4, 5, 6, 7, 8, 10, 11, 12, 13, 14, 15, 16, 17, 18, 19, 20, 21, 22, 23, 24, 25, 26 or 27 contiguous amino acids from the sequence of SEQ ID NO: 26.

In some embodiments, the CD28 transmembrane domain is encoded by a nucleotide sequence of at least 6, 9, 12, 15, 18, 21, 24, 27, 30, 33, 36, 39, 42, 45, 48, 51, 54, 57, 60, 63, 66, 69, 72, 75, 78 or 81 nucleotides from the sequence of SEQ ID NO: 27 or from a sequence having at least about 70%, 75%, 80%, 85%, 90%, 91%, 92%, 93%, 94%, 95%, 96%, 97%, 98% or 99% identity with SEQ ID NO: 27, e.g., at least 6, 9, 12, 15, 18, 21, 24, 27, 30, 33, 36, 39, 42, 45, 48, 51, 54, 57, 60, 63, 66, 69, 72, 75, 78 or 81 contiguous nucleotides from the sequence of SEQ ID NO: 27.

In some embodiments of the invention, the chimeric receptor may comprise a combination of at least two transmembrane domains, e.g., selected from a transmembrane domain of TNFR2, a transmembrane domain of CD8 and a transmembrane domain of CD28. Said transmembrane domains may be entire transmembrane domains or fragments or variants thereof, and may be linked to each other in a random or in a specified order. In certain embodiments, the combination of the at least two transmembrane domains or fragments or variants thereof comprise at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In some embodiments, the chimeric receptor comprises the amino acid sequence of a TNFR2 transmembrane domain (e.g., comprising or consisting of the amino acid sequence of SEQ ID NO: 22) or a fragment or variant thereof and the amino acid sequence of a CD8 transmembrane domain (e.g., comprising or consisting of the amino acid sequence of SEQ ID NO: 24) or a fragment or variant thereof. In certain embodiments, the chimeric receptor comprises a fusion transmembrane domain comprising the amino acid sequences of SEQ ID NOs: 59 and 62, SEQ ID NOs: 60 and 63, or SEQ ID NOs: 61 and 64.

In some embodiments, the chimeric receptor comprises the amino acid sequence of a TNFR2 transmembrane domain (e.g., comprising or consisting of the amino acid sequence of SEQ ID NO: 22) or a fragment or variant thereof and the amino acid sequence of a CD28 transmembrane domain (e.g., comprising or consisting of the amino acid sequence of SEQ ID NO: 26) or a fragment or variant thereof.

In some embodiments, the chimeric receptor comprises the amino acid sequence of CD8 transmembrane domain (e.g., comprising or consisting of the amino acid sequence of SEQ ID NO: 24) or a fragment or variant thereof and the amino acid sequence of a CD28 transmembrane domain (e.g., comprising or consisting of the amino acid sequence of SEQ ID NO: 26) or a fragment or variant thereof.

In some embodiments of the invention, the chimeric receptor may comprise a combination of at least three transmembrane domains, e.g., selected from a transmembrane domain of TNFR2, a transmembrane domain of CD8 and a transmembrane domain of CD28. Said transmembrane domains may be entire transmembrane domains or a fragment or variant thereof. In certain embodiments, the combination of the at least three transmembrane domains, a fragment or variant thereof comprise at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In some embodiments, the chimeric receptor comprises the amino acid sequence of a TNFR2 transmembrane domain (e.g., comprising or consisting of the amino acid sequence of SEQ ID NO: 22) or a fragment or variant thereof, the amino acid sequence of a CD8 transmembrane domain e.g., comprising or consisting of the amino acid sequence of (SEQ ID NO: 24) or a fragment or variant thereof and the amino acid sequence of a CD28 transmembrane domain (e.g., comprising or consisting of the amino acid sequence of SEQ ID NO: 26) or a fragment or variant thereof.

Thus, in some embodiments, the nucleic acid sequence encoding the transmembrane domain of a CAR of the invention comprises the nucleic acid sequence of a TNFR2 transmembrane domain (e.g., comprising or consisting of the amino acid sequence of SEQ ID NO: 23) or a fragment or variant thereof, and/or the nucleic acid sequence of a CD8 transmembrane domain (e.g., comprising or consisting of the amino acid sequence of SEQ ID NO: 25) or a fragment or variant thereof, and/or the nucleic acid sequence of a CD28 transmembrane domain (e.g., comprising or consisting of the amino acid sequence of SEQ ID NO: 27) or a fragment or variant thereof.

In some embodiments, the transmembrane domain of a CAR of the invention comprises the amino acid sequence of a TNFR2 transmembrane domain (e.g., comprising or consisting of the amino acid sequence of SEQ ID NO: 22) or a fragment or variant thereof, and/or the amino acid sequence of a CD8 transmembrane domain (e.g., comprising or consisting of the amino acid sequence of SEQ ID NO: 24) or a fragment or variant thereof, and/or the amino acid sequence of a CD28 transmembrane domain (e.g., comprising or consisting of the amino acid sequence of SEQ ID NO: 26) or a fragment or variant thereof; wherein the sequences comprised in the transmembrane domain are expressed in the same frame and as a single polypeptide chain.

In some embodiments, the transmembrane domain of a CAR of the invention comprises at least two different domains (e.g., a TNFR2 domain or a fragment or variant thereof and at least one other transmembrane domain (e.g., a CD8 or CD28 transmembrane domain) or a fragment or variant thereof) that may be linked to each other in a random or in a specified order.

Optionally, a short oligo- or polypeptide linker, for example, between 2 and 10 amino acids (e.g., 2, 3, 4, 5, 6, 7, 8, 9, or 10 amino acids) in length may form the linkage between distinct transmembrane domains.

In some embodiments, the transmembrane domain may be recombinant. In certain embodiments, the recombinant transmembrane domain comprises predominantly hydrophobic amino acids such as valine or leucine.

### IV. Intracellular Domains

In some embodiments, the intracellular domain of a CAR of the invention comprises a T cell primary signaling domain (or a sequence derived therefrom) and optionally one or more intracellular domain(s) of a T cell costimulatory molecule (or sequence(s) derived therefrom).

In some embodiments, the intracellular domain may comprise the entire intracellular portion, or the entire native intracellular signaling domain, of the molecule from which it is derived, or a functional fragment or variant thereof.

In some embodiments, the intracellular signaling domain consists of at least one primary signaling domain (e.g., a T cell primary signaling domain) or a fragment or variant thereof.

In some embodiments, the intracellular signaling domain consists of at least one costimulatory signaling domain (e.g., a T cell costimulatory molecule intracellular domain) or a fragment or variant thereof.

In some embodiments, the intracellular signaling domain comprises one or more intracellular domain(s) of a T cell costimulatory molecule or a fragment or variant thereof. In some embodiments, the intracellular signaling domain consists of one or more intracellular domain(s) of a T cell costimulatory molecule or a fragment or variant thereof.

In another embodiment, the intracellular signaling domain of the CAR of the invention comprises at least one costimulatory domain or a fragment or variant thereof and at least one primary signaling domain or a fragment or variant thereof.

In another embodiment, the intracellular signaling domain of the CAR of the invention consists of one costimulatory domain or a fragment or variant thereof and one primary signaling domain or a fragment or variant thereof.

In some embodiments, the intracellular signaling domain of a CAR of the invention comprises at least one, two, three, or four costimulatory domains or a fragment or variant thereof and at least one primary signaling domain or a fragment or variant thereof. In certain embodiments, one or more of the costimulatory domains are intracellular domains of a T cell costimulatory molecule. In certain embodiments, the at least one primary signaling domain is a T cell primary signaling domain.

In some embodiments of the invention, the primary signaling domain comprises a signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP1, and sequences derived therefrom.

In some embodiments, the primary signaling domain is a T cell primary signaling domain that comprises or consists of at least one functional signaling domain of CD3 zeta or a fragment or variant thereof.

In some embodiments, the T cell primary signaling domain comprises or consists of the CD3 zeta amino acid sequence of SEQ ID NO: 28, 29, 30 or 31, or an amino acid sequence with at least about 70%, 75%, 80%, 85%, 90%, 91%, 92%, 93%, 94%, 95%, 96%, 97%, 98% or 99% identity to SEQ ID NO: 28, 29, 30 or 31.

In some embodiments, the CD3 zeta primary signaling domain comprises or consists of an amino acid sequence having at least one, two or three modifications but not more than 20, 10 or 5 modifications, compared to an amino acid sequence of SEQ ID NO: 28, 29, 30 or 31, or an amino acid sequence with at least about 70%, 75%, 80%, 85%, 90%, 91%, 92%, 93%, 94%, 95%, 96%, 97%, 98% or 99% identity to SEQ ID NO: 28, 29, 30 or 31.

Thus, in some embodiments, the nucleic acid sequence encoding the T cell primary signaling domain comprises or consists of the CD3 zeta domain nucleic acid sequence of SEQ ID NO: 32 or SEQ ID NO: 33, or a nucleotide sequence with at least about 70%, 75%, 80%, 85%, 90%, 91%, 92%, 93%, 94%, 95%, 96%, 97%, 98% or 99% identity to SEQ ID NO: 32 or SEQ ID NO: 33.

In some embodiments, the CD3 zeta primary signaling domain comprises at least 2, 10, 20, 30, 40, 50, 60, 70, 80, 90, 100, 110 or 112 amino acids from the sequence of SEQ ID NO: 28, 29, 30 or 31, or from a sequence having at least about 70% identity with SEQ ID NO: 28, 29, 30 or 31, e.g., at least 2, 10, 20, 30, 40, 50, 60, 70, 80, 90, 100, 110 or 112 contiguous amino acids from SEQ ID NO: 28, 29, 30 or 31.

In some embodiments, the CD3 zeta primary signaling domain is encoded by a nucleotide sequence of at least 6, 30, 60, 90, 120, 150, 180, 210, 240, 270, 300, 330 or 336 nucleotides from the sequence of SEQ ID NO: 32 or SEQ ID NO: 33, or from a sequence having at least about 70%, 75%, 80%, 85%, 90%, 91%, 92%, 93%, 94%, 95%, 96%, 97%, 98% or 99% identity with SEQ ID NO: 32 or SEQ ID NO: 33, e.g., at least 6, 30, 60, 90, 120, 150, 180, 210, 240, 270, 300, 330 or 336 contiguous nucleotides from SEQ ID NO: 32 or SEQ ID NO: 33.

In some embodiments, T cell primary signaling domains that act in a stimulatory manner may comprise signaling motifs known as immunoreceptor tyrosine-based activation motifs (ITAMS). Examples of ITAM-containing T cell primary intracellular signaling domains that are of particular use in the invention include, but are not limited to, those of (or that are derived from) CD3 zeta, common FcR gamma (FCER1G), Fc gamma RIIa, FcR beta (Fc Epsilon Rib), CD3 gamma, CD3 delta, CD3 epsilon, CD5, CD22, CD66b, CD79a, CD79b, DAP10, and DAP12.

In some embodiments, the T cell primary signaling domain comprises a modified ITAM domain, e.g., a mutated ITAM domain that has altered (e.g., increased or decreased) activity as compared to the native ITAM domain. In some embodiments, a primary signaling domain comprises a modified ITAM-containing primary intracellular signaling domain, e.g., an optimized and/or truncated ITAM-containing primary intracellular signaling domain. In certain embodiments, a primary signaling domain may comprise one, two, three, four or more ITAM motifs.

In some embodiments, the intracellular signaling domain of a CAR of the invention comprises a T cell primary signaling domain (such as, for example, a CD3 zeta signaling domain or a fragment or variant thereof), combined with one or more costimulatory signaling domains, wherein said costimulatory signaling domains are entire costimulatory intracellular signaling domains or a fragment or variant thereof.

Examples of intracellular domains of a T cell costimulatory molecule include, but are not limited to, the signaling domains of proteins selected from the group consisting of TNFR2 (CD120b/TNFRSF1B), 4-1BB (CD137), ICOS (CD278), CD27, CD28, CTLA-4 (CD152), PD-1, an MHC class I molecule, BTLA, a Toll ligand receptor, OX40, CD30, CD40, lymphocyte function-associated antigen-1 (LFA-1), CD2, CD7, LIGHT, NKG2C, B7-H3, a ligand that specifically binds with CD83, CDS, ICAM-1, GITR, ARHR, BAFFR, HVEM (LIGHTR), SLAMF7, NKp80 (KLRF1), NKp44, NKp30, NKp46, CD160 (BY55), CD19, CD19a, CD4, CD8alpha, CD8beta, IL2ra, IL6Ra, IL2R beta, IL2R gamma, IL7R alpha, IL-13RA1/RA2, IL-33R (IL1RL1), IL-10RA/RB, IL-4R, IL-5R (CSF2RB), IL-21R, ITGA4, VLA1, CD49a, ITGA4, IA4, CD49D, ITGA6, VLA-6, CD49f, ITGAD, CD11d, ITGAE, CD103, ITGAL, CD11a/CD18, ITGAM, CD11b, ITGAX, CD11c, ITGB1, CD29, ITGB2, CD18, ITGB7, NKG2D, NKG2C, CD95, TNFR1 (CD120a/TNFRSF1A), TGFbR1/2/3, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, common gamma chain, a ligand that specifically binds with CD83, NKp44, NKp30, NKp46, NKG2D, and any combination thereof.

In some embodiments, the chimeric receptor comprises at least one intracellular domain of a T cell costimulatory molecule selected from the group consisting of TNFR2, 4-1BB, ICOS, CD27, OX40, CD28, CTLA4 and PD-1.

In some embodiments, the chimeric receptor comprises at least one costimulatory signaling domain, wherein said costimulatory signaling domain is an entire costimulatory signaling domain or a fragment or variant thereof.

In some embodiments, the T cell costimulatory signaling domain comprises or consists of the amino acid sequence of a TNFR2 costimulatory intracellular signaling domain (e.g., comprising or consisting of the amino acid sequence of SEQ ID NO: 34) or an amino acid sequence with at least about 70%, 75%, 80%, 85%, 90%, 91%, 92%, 93%, 94%, 95%, 96%, 97%, 98% or 99% identity to SEQ ID NO: 34. In some embodiments, the costimulatory signaling domain comprises or consists of an amino acid sequence having at least one, two or three modifications but not more than 20, 10 or 5 modifications compared to an amino acid sequence of SEQ ID NO: 34.

In some embodiments, the T cell costimulatory signaling domain is encoded by the nucleotide sequence of a TNFR2 costimulatory intracellular signaling domain (e.g., comprising or consisting of the amino acid sequence of SEQ ID NO: 35), or a nucleotide sequence with at least 70%, 75%, 80%, 85%, 90%, 91%, 92%, 93%, 94%, 95%, 96%, 97%, 98% or 99% identity to SEQ ID NO: 35.

In some embodiments, the TNFR2 intracellular costimulatory signaling domain comprises at least 2, 6, 10, 20, 30, 40, 50, 60, 70, 80, 90, 100, 110, 120, 130, 140, 150, 160, 170 or 174 amino acids from the sequence of SEQ ID NO: 34 or from a sequence having at least about 70%, 75%, 80%, 85%, 90%, 91%, 92%, 93%, 94%, 95%, 96%, 97%, 98% or 99% identity with SEQ ID NO: 34, e.g., at least 2, 10, 20, 30, 40, 50, 60, 70, 80, 90, 100, 110, 120, 130, 140, 150, 160, 170 or 174 contiguous amino acids from SEQ ID NO: 34.

In some embodiments, the TNFR2 intracellular costimulatory signaling domain is encoded by a nucleotide sequence of at least 6, 18, 30, 60, 90, 120, 150, 180, 210, 240, 260, 270, 300, 330, 360, 390, 420, 450, 480, 510 or 522 nucleotides from the sequence of SEQ ID NO: 35 or from a sequence having at least about 70%, 75%, 80%, 85%, 90%, 91%, 92%, 93%, 94%, 95%, 96%, 97%, 98% or 99% identity with SEQ ID NO:35, e.g., at least 6, 18, 30, 60, 90, 120, 150, 180, 210, 240, 260, 270, 300, 330, 360, 390, 420, 450, 480, 510 or 522 contiguous nucleotides from SEQ ID NO: 35.

In some embodiments, the intracellular costimulatory signaling domain comprises domains I and II, domains I-III, domains I-IV, or domains I-V of the TNFR2 intracellular costimulatory signaling domain (e.g., SEQ ID NO: 34). In certain embodiments, the intracellular costimulatory signaling domain comprises domains I and II of the TNFR2 intracellular costimulatory domain.

In some embodiments the intracellular costimulatory signaling domain comprises residues 1-20 (Δ151), 1-70 (Δ104), 1-115 (Δ59), or 1-156 (Δ18) of SEQ ID NO: 34.

In some embodiments, the T cell costimulatory signaling domain comprises or consists of the amino acid sequence of a 4-1BB costimulatory intracellular signaling domain (e.g., comprising or consisting of the amino acid sequence of SEQ ID NO: 36) or an amino acid sequence with at least about 70%, 75%, 80%, 85%, 90%, 91%, 92%, 93%, 94%, 95%, 96%, 97%, 98% or 99% identity to SEQ ID NO: 36. In some embodiments, the T cell costimulatory signaling domain comprises or consists of an amino acid sequence having at least one, two or three modifications but not more than 20, 10 or 5 modifications compared to the amino acid sequence of SEQ ID NO: 36.

In some embodiments, the T cell costimulatory signaling domain is encoded by a 4-1BB costimulatory intracellular signaling domain nucleotide sequence (e.g., comprising or consisting of the amino acid sequence of SEQ ID NO: 37), or a nucleotide sequence with at least about 70%, 75%, 80%, 85%, 90%, 91%, 92%, 93%, 94%, 95%, 96%, 97%, 98% or 99% identity to SEQ ID NO: 37.

In some embodiments, the 4-1BB costimulatory intracellular signaling domain comprises at least 2, 3, 6, 9, 12, 15, 18, 21, 24, 27, 30, 33, 36, 39, or 42 amino acids from the sequence of SEQ ID NO: 36 or from a sequence having at least about 70%, 75%, 80%, 85%, 90%, 91%, 92%, 93%, 94%, 95%, 96%, 97%, 98% or 99% identity with SEQ ID NO: 36, e.g., at least 2, 3, 6, 9, 12, 15, 18, 21, 24, 27, 30, 33, 36, 39, or 42 contiguous amino acids from SEQ ID NO: 36.

In some embodiments, the 4-1BB costimulatory intracellular signaling domain is encoded by a nucleotide sequence of at least 6, 18, 27, 36, 45, 54, 63, 72, 81, 96, 99, 108, 117 or 126 nucleotides from the sequence of SEQ ID NO: 37 or from a sequence having at least about 70%, 75%, 80%, 85%, 90%, 91%, 92%, 93%, 94%, 95%, 96%, 97%, 98% or 99% identity with SEQ ID NO: 37, e.g., at least 6, 18, 27, 36, 45, 54, 63, 72, 81, 96, 99, 108, 117 or 126 contiguous nucleotides from SEQ ID NO: 37.

In some embodiments, the T cell costimulatory signaling domain comprises or consists of the amino acid sequence of a CD27 costimulatory intracellular signaling domain (e.g., comprising or consisting of the amino acid sequence of SEQ ID NO: 38) or an amino acid sequence with at least about 70%, 75%, 80%, 85%, 90%, 91%, 92%, 93%, 94%, 95%, 96%, 97%, 98% or 99% identity to SEQ ID NO: 38. In some embodiments, the T cell costimulatory signaling domain comprises or consists of an amino acid sequence having at least one, two or three modifications but not more than 20, 10 or 5 modifications compared to the amino acid sequence of SEQ ID NO: 38.

In some embodiments, the T cell costimulatory signaling domain is encoded by a CD27 costimulatory intracellular signaling domain nucleotide sequence (e.g., comprising or consisting of the amino acid sequence of SEQ ID NO: 39), or a nucleotide sequence with at least about 70%, 75%, 80%, 85%, 90%, 91%, 92%, 93%, 94%, 95%, 96%, 97%, 98% or 99% identity to SEQ ID NO: 39.

In some embodiments, the CD27 costimulatory intracellular signaling domain comprises at least 2, 3, 6, 9, 12, 15, 18, 21, 24, 27, 30, 33, 36, 39, 42, 45 or 48 amino acids from the sequence of SEQ ID NO: 38 or from a sequence having at least about 70%, 75%, 80%, 85%, 90%, 91%, 92%, 93%, 94%, 95%, 96%, 97%, 98% or 99% identity with SEQ ID NO: 38, e.g., at least 2, 3, 6, 9, 12, 15, 18, 21, 24, 27, 30, 33, 36, 39, 42, 45 or 48 contiguous amino acids from SEQ ID NO: 38.

In some embodiments, the CD27 costimulatory intracellular signaling domain is encoded by a nucleotide sequence of at least 6, 18, 27, 36, 45, 54, 63, 72, 81, 96, 99, 108, 117, 126, 135 or 144 nucleotides from the sequence of SEQ ID NO: 39 or from a sequence having at least about 70%, 75%, 80%, 85%, 90%, 91%, 92%, 93%, 94%, 95%, 96%, 97%, 98% or 99% identity with SEQ ID NO: 39, e.g., at least 6, 18, 27, 36, 45, 54, 63, 72, 81, 96, 99, 108, 117, 126, 135 or 144 contiguous nucleotides from SEQ ID NO: 39.

In some embodiments, the T cell costimulatory signaling domain comprises or consists of the amino acid sequence of a CD28 costimulatory intracellular signaling domain (e.g., comprising or consisting of the amino acid sequence of SEQ ID NO: 40) or an amino acid sequence with at least about 70%, 75%, 80%, 85%, 90%, 91%, 92%, 93%, 94%, 95%, 96%, 97%, 98% or 99% identity to SEQ ID NO: 40. In some embodiments, the T cell costimulatory signaling domain comprises or consists of an amino acid sequence having at least one, two or three modifications but not more than 20, 10 or 5 modifications compared to the amino acid sequence of SEQ ID NO: 40.

In some embodiments, the T cell costimulatory signaling domain is encoded by a CD28 costimulatory intracellular signaling domain nucleotide sequence (e.g., comprising or consisting of SEQ ID NO: 41), or a nucleotide sequence with at least about 70%, 75%, 80%, 85%, 90%, 91%, 92%, 93%, 94%, 95%, 96%, 97%, 98% or 99% identity to SEQ ID NO: 41.

In some embodiments, the CD28 costimulatory intracellular signaling domain comprises at least 2, 3, 6, 9, 12, 15, 18, 21, 24, 27, 30, 33, 36, 39 or 41 amino acids from the sequence of SEQ ID NO: 40 or from a sequence having at least about 70%, 75%, 80%, 85%, 90%, 91%, 92%, 93%, 94%, 95%, 96%, 97%, 98% or 99% identity with SEQ ID NO: 40, e.g., at least 2, 3, 6, 9, 12, 15, 18, 21, 24, 27, 30, 33, 36, 39 or 41 contiguous amino acids from SEQ ID NO: 40.

In some embodiments, the CD28 costimulatory intracellular signaling domain is encoded by a nucleotide sequence of at least 6, 18, 27, 36, 45, 54, 63, 72, 81, 96, 99, 108, 117 or 123 nucleotides from the sequence of SEQ ID NO: 41 or from a sequence having at least about 70%, 75%, 80%, 85%, 90%, 91%, 92%, 93%, 94%, 95%, 96%, 97%, 98% or 99% identity with SEQ ID NO: 41, e.g., at least 6, 18, 27, 36, 45, 54, 63, 72, 81, 96, 99, 108, 117 or 123 contiguous nucleotides from SEQ ID NO: 41.

In some embodiments of the invention, the chimeric receptor comprises a combination of at least two intracellular domains of a T cell costimulatory molecule In certain embodiments, the at least two intracellular domains may be selected from an intracellular domain of TNFR2, an intracellular domain of 4-1BB, an intracellular domain of CD27 and an intracellular domain of CD28. In particular embodiments, the said costimulatory intracellular signaling domains are entire costimulatory intracellular signaling domains or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a 4-1BB costimulatory intracellular signaling domain (e.g., comprising or consisting of the amino acid sequence of SEQ ID NO: 36) or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a CD27 costimulatory intracellular signaling domain (e.g., comprising or consisting of the amino acid sequence of SEQ ID NO: 38) or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments, the chimeric receptor comprises the amino acid sequence of a 4-1BB costimulatory intracellular signaling domain (e.g., comprising or consisting of the amino acid sequence of SEQ ID NO: 36) or a fragment or variant thereof and the amino acid sequence of a CD27 costimulatory intracellular signaling domain (e.g., comprising or consisting of the amino acid sequence of SEQ ID NO: 38) or a fragment or variant thereof.

In some embodiments, the chimeric receptor comprises the amino acid sequence of a 4-1BB costimulatory intracellular signaling domain (e.g., comprising or consisting of the amino acid sequence of SEQ ID NO: 36) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments, the chimeric receptor comprises the amino acid sequence of a CD27 costimulatory intracellular signaling domain (e.g., comprising or consisting of the amino acid sequence of SEQ ID NO: 38) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments of the invention, the chimeric receptor comprises a combination of at least three intracellular domains of a T cell costimulatory molecule, e.g., selected from an intracellular domain of TNFR2, an intracellular domain of 4-1BB, an intracellular domain of CD27 and an intracellular domain of CD28.

In some embodiments of the invention, the chimeric receptor may comprise at least three costimulatory intracellular signaling domains, wherein said domains are entire costimulatory intracellular signaling domains or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a 4-1BB costimulatory intracellular signaling domain (e.g., comprising or consisting of the amino acid sequence of SEQ ID NO: 36) or a fragment or variant thereof and the amino acid sequence of a CD27 costimulatory intracellular signaling domain (e.g., comprising or consisting of the amino acid sequence of SEQ ID NO: 38) or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a 4-1BB costimulatory intracellular signaling domain (e.g., comprising or consisting of the amino acid sequence of SEQ ID NO: 36) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a CD27 costimulatory intracellular signaling domain (e.g., comprising or consisting of the amino acid sequence of SEQ ID NO: 38) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments, the chimeric receptor comprises the amino acid sequence of a 4-1BB costimulatory intracellular signaling domain (e.g., comprising or consisting of the amino acid sequence of SEQ ID NO: 36) or a fragment or variant thereof and the amino acid sequence of a CD27 costimulatory intracellular signaling domain e.g., comprising or consisting of the amino acid sequence of SEQ ID NO: 38) or a fragment or variant thereof and the amino acid sequence of a CD28 costimulatory intracellular signaling domain (e.g., comprising or consisting of the amino acid sequence of SEQ ID NO: 40) or a fragment or variant thereof.

In some embodiments of the invention, the chimeric receptor comprises a combination of at least four intracellular domains of a T cell costimulatory molecule, e.g., selected from an intracellular domain of TNFR2, an intracellular domain of 4-1BB, an intracellular domain of CD27 and an intracellular domain of CD28.

In some embodiments of the invention, the chimeric receptor may comprise a combination of at least four intracellular domains of a T cell costimulatory molecule wherein said costimulatory intracellular signaling domains are entire costimulatory intracellular signaling domains or a fragment or variant thereof.

In some embodiments, the chimeric receptor comprises the amino acid sequence of a TNFR2 costimulatory intracellular signaling domain (e.g., comprising or consisting of the amino acid sequence of SEQ ID NO: 34) or a fragment or variant thereof and the amino acid sequence of a 4-1BB costimulatory intracellular signaling domain (e.g., comprising or consisting of the amino acid sequence of SEQ ID NO: 36) or a fragment or variant thereof, and the amino acid sequence of a CD27 costimulatory intracellular signaling domain (e.g., comprising or consisting of the amino acid sequence of SEQ ID NO: 38) or a fragment or variant thereof, and the amino acid sequence of a CD28 costimulatory intracellular signaling domain (e.g., comprising or consisting of the amino acid sequence of SEQ ID NO: 40) or a fragment or variant thereof.

Thus, in some embodiments, the nucleic acid sequence encoding the T cell costimulatory signaling domain comprises the nucleic acid sequence of a TNFR2 costimulatory intracellular signaling domain (e.g., comprising or consisting of the amino acid sequence of SEQ ID NO: 35) or a fragment or variant thereof, and/or the nucleic acid sequence of a 4-1BB costimulatory intracellular signaling domain (e.g., comprising or consisting of the amino acid sequence of SEQ ID NO: 37) or a fragment or variant thereof, and/or the nucleic acid sequence of a CD27 costimulatory intracellular signaling domain (e.g., comprising or consisting of the amino acid sequence of SEQ ID NO: 39) or a fragment or variant thereof, and/or the nucleic acid sequence of a CD28 costimulatory intracellular signaling domain (e.g., comprising or consisting of the amino acid sequence of SEQ ID NO: 41) or a fragment or variant thereof.

In some embodiments, the intracellular signaling domain of a CAR of the invention comprises:
a TNFR2 costimulatory intracellular signaling domain with the amino acid sequence of SEQ ID NO: 34, or a fragment or variant thereof, and/or a 4-1BB costimulatory intracellular signaling domain with the amino acid sequence of SEQ ID NO: 36, or a fragment or variant thereof, and/or the a CD27 costimulatory intracellular signaling domain with the amino acid sequence of SEQ ID NO: 38, and/or a CD28 costimulatory intracellular signaling domain with the amino acid sequence of SEQ ID NO: 40; and/or
a CD3 zeta primary intracellular signaling domain with the amino acid sequence of SEQ ID NO: 28, 29, 30 or 31, or a fragment or variant thereof, wherein the sequences comprised in the intracellular domain are expressed in the same frame and as a single polypeptide chain.

Thus, in some embodiments, the nucleic acid sequence encoding the intracellular signaling domain of a CAR of the invention comprises:
a TNFR2 costimulatory intracellular signaling domain nucleic acid sequence of SEQ ID NO: 35 or a fragment or variant thereof, and/or a 4-1BB costimulatory intracellular signaling domain nucleic acid sequence of SEQ ID NO: 37 or a fragment or variant thereof, and/or a CD27 costimulatory intracellular signaling domain nucleic acid sequence of SEQ ID NO: 39 or a fragment or variant thereof, and/or a CD28 costimulatory intracellular signaling domain nucleic acid sequence of SEQ ID NO: 41 or a fragment or variant thereof; and/or
a CD3 zeta primary intracellular signaling domain of SEQ ID NO: 32 or SEQ ID NO: 33 or a fragment or variant thereof.

In some embodiments, the intracellular signaling domain of a CAR of the invention comprises at least two different domains (e.g., a primary signaling domain or a fragment or variant thereof and at least one intracellular domain of a T cell costimulatory molecule or a fragment or variant thereof) that may be linked to each other in a random or in a specified order.

Optionally, a short oligo- or polypeptide linker, for example, between 2 and 10 amino acids (e.g., 2, 3, 4, 5, 6, 7, 8, 9, or 10 amino acids) in length may form the linkage between distinct signaling domains. In some embodiments, a glycine-serine doublet (GS) is used as a suitable linker. In some embodiments, a single amino acid, e.g., an alanine (A), a glycine (G), is used as a suitable linker. Other examples of linker are described herein.

In some embodiments, the intracellular signaling domain of a CAR of the invention comprises two or more (e.g., 2, 3, 4, 5, or more) costimulatory intracellular signaling domains. In some embodiments, any or all of the two or more (e.g., 2, 3, 4, 5, or more) costimulatory signaling domains are separated by a linker molecule, e.g., a linker molecule as described herein.

In some embodiments, the intracellular signaling domain of a chimeric receptor of the invention comprises the primary intracellular signaling domain of CD3 zeta (e.g., SEQ ID NO: 28, 29, 30 or 31) and the costimulatory intracellular signaling domain of TNFR2 (e.g., SEQ ID NO: 34).

In some embodiments, the intracellular signaling domain of a chimeric receptor of the invention comprises the primary intracellular signaling domain of CD3 zeta (e.g., SEQ ID NO: 28, 29, 30 or 31) and the costimulatory intracellular signaling domain of 4-1BB (e.g., SEQ ID NO: 36).

In some embodiments, the intracellular signaling domain of a chimeric receptor of the invention comprises the primary intracellular signaling domain of CD3 zeta (e.g., SEQ ID NO: 28, 29, 30 or 31) and the costimulatory intracellular signaling domain of CD27 (e.g., SEQ ID NO: 38).

In some embodiments, the intracellular signaling domain of a chimeric receptor of the invention comprises the primary intracellular signaling domain of CD3 zeta (e.g., SEQ ID NO: 28, 29, 30 or 31) and the costimulatory intracellular signaling domain of CD28 (e.g., SEQ ID NO: 40).

In some embodiments, a CAR of the invention comprises any combination of an extracellular binding domain as described herein, a transmembrane domain as described herein, an intracellular signaling domain as described herein, and optionally a spacer or hinge domain as described herein.

In some embodiments, a CAR of the invention further comprises a leader sequence located N-terminally from the specific extracellular binding domain. A non-limiting example is a leader sequence of CD8 that may comprise or consist of the sequence of SEQ ID NO: 42.

In some embodiments, a CAR of the invention further comprises a tag, such as, for example, a tag for quality control, enrichment, tracking in vivo and the like. Said tag may be localized N-terminally, C-terminally and/or internally. Examples of tags that may be used in a CAR of the invention are well known by the skilled artisan. For example, but without limitation, a tag used in the invention can be a tag selected from the group consisting of streptavidin tag (e.g., SEQ ID NO: 47), hemagglutinin tag, poly arginine tag, poly histidine tag, Myc tag, strep tag, S-tag, HAT tag, 3× flag tag, calmodulin-binding peptide tag, SBP tag, chitin binding domain tag, GST tag, maltose-binding protein tag, fluorescent protein tag, T7 tag, V5 tag and Xpress tag. Other examples of tags include, without limitation, NWSHPQFEK (SEQ ID NO: 43) or SAWSHPQFEK (SEQ ID NO: 44).

In some embodiments, a CAR of the invention further comprises P2A (SEQ ID NO: 45) and/or GFP (SEQ ID NO: 46) sequences.

In some embodiments, a CAR of the invention comprises:
at least one extracellular binding domain,
optionally an extracellular hinge domain,
at least one TNFR2 transmembrane domain or a fragment or variant thereof, and
at least one intracellular primary signaling domain or a fragment or variant thereof
In some embodiments, a CAR of the invention comprises:
at least one extracellular binding domain;
at least one TNFR2 transmembrane domain (e.g., SEQ ID NO: 22) or a fragment or variant thereof; and
at least one CD3 zeta primary intracellular signaling domain (e.g., SEQ ID NO: 28, 29, 30 or 31) or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof
at least one transmembrane domain of TNFR2 (e.g., SEQ ID NO: 22) or a fragment or variant thereof; and
at least one CD3 zeta primary intracellular signaling domain (e.g., SEQ ID NO: 28, 29, 30 or 31) or a fragment or variant thereof. In particular embodiments, the at least one hinge domain is a CD8 hinge domain (e.g., SEQ ID NO: 14)

In some embodiments, a CAR of the invention comprises:
at least one extracellular binding domain,
optionally at least one extracellular hinge domain,
at least one transmembrane domain or a fragment or variant thereof, and
at least one costimulatory intracellular signaling domain or a fragment or variant thereof,
   wherein at least one of the transmembrane domain and costimulatory intracellular signaling domain is a TNFR2 transmembrane or a TNFR2 costimulatory intracellular domain or a fragment or variant thereof.

In some embodiments, a CAR of the invention comprises:
at least one extracellular binding domain;
at least one transmembrane domain selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof; and
at least one costimulatory intracellular signaling domain selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof,
   wherein at least one of the transmembrane domain and costimulatory intracellular signaling domain is a TNFR2 transmembrane or TNFR2 costimulatory intracellular signaling domain or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least one transmembrane domain selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof; and
at least one costimulatory intracellular signaling domain selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof,
   wherein at least one of the transmembrane domain and costimulatory intracellular signaling domain is a TNFR2 transmembrane or a TNFR2 costimulatory intracellular signaling domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
optionally at least one extracellular hinge domain,
at least one transmembrane domain or a fragment or variant thereof,
at least one intracellular costimulatory signaling domain or a fragment or variant thereof, and
at least one T cell primary signaling intracellular domain or a fragment or variant thereof,
   wherein at least one of the transmembrane domain and costimulatory intracellular signaling domain is a TNFR2 transmembrane or a TNFR2 costimulatory intracellular signaling domain or a fragment or variant thereof.

In some embodiments, a CAR of the invention comprises:
at least one extracellular binding domain;
at least one transmembrane domain selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least one costimulatory intracellular signaling domain selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
at least one CD3 zeta primary intracellular signaling domain (e.g., SEQ ID NO: 28, 29, 30 or 31) or a fragment or variant thereof;
   wherein at least one of the transmembrane domain and costimulatory intracellular signaling domain is a TNFR2 transmembrane or a TNFR2 costimulatory intracellular signaling domain or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least one costimulatory intracellular signaling domain selected from the group consisting of TNFR2 intracellular domain (e.g., SEQ ID NO: 34) or a fragment or variant thereof, 4-1BB intracellular domain (e.g., SEQ ID NO 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
at least one CD3 zeta primary intracellular signaling domain (e.g., SEQ ID NO: 28, 29, 30 or 31) or a fragment or variant thereof;
   wherein at the least one of the transmembrane domain and costimulatory intracellular signaling domain is a TNFR2 transmembrane or a TNFR2 costimulatory intracellular signaling domain or a fragment or variant thereof. In particular embodiments, the at least one hinge domain is a CD8 hinge domain (e.g., SEQ ID NO: 14).

According to some embodiments, a CAR of the invention comprises:
at least one extracellular binding domain,
optionally at least one extracellular hinge domain,
at least one transmembrane domain or a fragment or variant thereof,
at least two intracellular costimulatory signaling domains or fragments or variants thereof, and
optionally at least one T cell primary signaling domain or a fragment or variant thereof, wherein at least one of the transmembrane domain and/or costimulatory intracellular signaling domains is a TNFR2 transmembrane or a TNFR2 costimulatory intracellular signaling domain or a fragment or variant thereof.

In some embodiments, a CAR of the invention comprises:
at least one extracellular binding domain;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least two costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or a TNFR2 costimulatory intracellular signaling domain or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least two costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or a TNFR2 costimulatory intracellular signaling domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
optionally at least one extracellular hinge domain,
at least one transmembrane domain or a fragment or variant thereof,
at least three costimulatory intracellular signaling domains or fragments or variants thereof, and
optionally at least one T cell primary intracellular signaling domain or a fragment or variant thereof,
   wherein at least one of the transmembrane domain and/or costimulatory intracellular signaling domains is a TNFR2 transmembrane or a TNFR2 costimulatory intracellular signaling domain or a fragment or variant thereof.

In some embodiments, a CAR of the invention comprise:
at least one extracellular binding domain;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least three costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or costimulatory intracellular signaling domain or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least three costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or costimulatory intracellular signaling domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
optionally at least one extracellular hinge domain,
at least one transmembrane domain or a fragment or variant thereof,
at least four costimulatory intracellular signaling domains or fragments or variants thereof, and
optionally at least one T cell primary intracellular signaling domain or a fragment or variant thereof,
   wherein at least one of the transmembrane domain and/or costimulatory intracellular signaling domains is a TNFR2 transmembrane or a TNFR2 costimulatory intracellular signaling domain or a fragment or variant thereof.

In some embodiments, a CAR of the invention comprises:
at least one extracellular binding domain;
at least one transmembrane domain selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least four costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or costimulatory intracellular signaling domain or a fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (SEQ ID NO: 16) or a fragment or variant thereof, and IgD (e.g., SEQ ID NO: 18) or fragments or variant thereof;
at least one transmembrane domain selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least four costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domain and/or costimulatory intracellular signaling domains is a TNFR2 transmembrane or a TNFR2 costimulatory signaling intracellular domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
optionally at least one extracellular hinge domain,
at least 1, 2 or 3 transmembrane domains or fragments or variants thereof, and
optionally at least one T cell primary intracellular signaling domain or a fragment or variant thereof,
   wherein at least one of the transmembrane domains is a TNFR2 transmembrane domain or a fragment or variant thereof.

In some embodiments, a CAR of the invention comprises:
at least one extracellular binding domain;
at least 1, 2 or 3 transmembrane domains selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof; and
optionally at least one CD3 zeta primary signaling domain (e.g., SEQ ID NO: 28, 29, 30 or 31) or a fragment or variant thereof;
   wherein at least one of the transmembrane domains is a TNFR2 transmembrane domain or fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least 1, 2 or 3 transmembrane domains selected from the group consisting of TNFR2 (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof; and
optionally at least one CD3 zeta primary intracellular signaling domain (e.g., SEQ ID NO: 28, 29, 30 or 31) or a fragment or variant thereof;
   wherein at least one of the transmembrane domains is a TNFR2 transmembrane domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
   optionally at least one extracellular hinge domain,
at least 1, 2 or 3 transmembrane domains or fragments or variants thereof,
at least 1, 2, 3 or 4 intracellular costimulatory signaling domains or fragments or variants thereof, and
optionally at least one T cell primary signaling domain or a fragment or variant thereof,
   wherein at least one of the transmembrane domains and/or costimulatory intracellular signaling domains is a TNFR2 transmembrane or a TNFR2 costimulatory intracellular signaling domain or a fragment or variant thereof.

In some embodiments, a CAR of the invention comprises:
at least one extracellular binding domain;
at least 1, 2 or 3 transmembrane domains selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, and CD28 transmembrane domain (e.g., SEQ ID NO: 26) or a fragment or variant thereof;
at least 1, 2, 3 or 4 costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary signaling domain (e.g., SEQ ID NO: 28, 29, 30 or 31) or a fragment or variant thereof;
   wherein at least one of the transmembrane domains and/or costimulatory intracellular signaling domains is a TNFR2 transmembrane or a TNFR2 costimulatory intracellular signaling domain or fragment or variant thereof.

In some embodiments, a CAR of the invention comprises:
at least one extracellular binding domain;
at least one hinge domain selected from the group consisting of CD8 (e.g., SEQ ID NO: 14) or a fragment or variant thereof, CD28 (e.g., SEQ ID NO: 20) or a fragment or variant thereof, IgG4 (e.g., SEQ ID NO: 16) or a fragment or variant thereof, and IgD (e.g., SEQ ID NO: 18) or a fragment or variant thereof;
at least 1, 2 or 3 transmembrane domains selected from the group consisting of TNFR2 transmembrane domain (e.g., SEQ ID NO: 22) or a fragment or variant thereof, CD8 transmembrane domain (e.g., SEQ ID NO: 24) or a fragment or variant thereof, or CD28 transmembrane domain (e.g., SEQ ID NO: 26) or a fragment or variant thereof;
at least 1, 2, 3 or 4 costimulatory intracellular signaling domains selected from the group consisting of TNFR2 intracellular domain (e.g., SEQ ID NO: 34) or a fragment or variant thereof, 4-1BB intracellular domain (e.g., SEQ ID NO: 36) or a fragment or variant thereof, CD27 intracellular domain (e.g., SEQ ID NO: 38) or a fragment or variant thereof, and CD28 intracellular domain (e.g., SEQ ID NO: 40) or a fragment or variant thereof; and
optionally at least one CD3 zeta primary signaling domain (e.g., SEQ ID NO: 28, 29, 30 or 31) or a fragment or variant thereof;
   wherein at least one of the transmembrane domains and/or costimulatory intracellular signaling domains is a TNFR2 transmembrane or a TNFR2 costimulatory intracellular signaling domain or a fragment or variant thereof. In particular embodiments, the at least one hinge domain is a CD8 hinge domain (e.g., SEQ ID NO: 14).

In some embodiments, a CAR of the invention comprises:
at least one extracellular binding domain,
optionally at least one extracellular hinge domain,
at least one transmembrane domain,
at least one intracellular domain, wherein the at least one intracellular domain comprises at least one primary intracellular signaling domain and optionally at least one costimulatory intracellular signaling domain,
   and wherein the at least one transmembrane domain is a human TNFR2 transmembrane domain or a fragment or variant thereof or any transmembrane domain or a fragment or variant thereof or a combination thereof, and/or the at least one costimulatory intracellular signaling domain is a human TNFR2 costimulatory intracellular signaling domain or a fragment or variant thereof or any costimulatory intracellular signaling domain or a fragment or variant thereof or a combination thereof, and wherein at least one of the transmembrane domain and costimulatory intracellular signaling domain is a TNFR2 transmembrane domain or a fragment or variant thereof or a TNFR2 costimulatory intracellular signaling domain or a fragment or variant thereof.

In some embodiments, a CAR of the invention comprises a sequence comprising a hinge region of human CD8, a transmembrane domain of human TNFR2, a costimulatory intracellular signaling domain of human TNFR2 and a primary intracellular signaling domain of human CD3ζ chain, wherein said sequence corresponds to the amino acid sequence of SEQ ID NO: 48 or an amino acid sequence with at least about 70%, 75%, 80%, 85%, 90%, 91%, 92%, 93%, 94%, 95%, 96%, 97%, 98% or 99% identity to SEQ ID NO: 48.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 48 or a sequence with at least about 70%, 75%, 80%, 85%, 90%, 91%, 92%, 93%, 94%, 95%, 96%, 97%, 98% or 99% identity to SEQ ID NO: 48.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 48 or a sequence with at least about 70%, 75%, 80%, 85%, 90%, 91%, 92%, 93%, 94%, 95%, 96%, 97%, 98% or 99% identity to SEQ ID NO: 48.

In some embodiments, a CAR of the invention comprises an anti-CD19 scFv (e.g., comprising or consisting of the binding domain sequence of SEQ ID NO: 1 or 2), linked to the amino acid sequence of SEQ ID NO: 48 or a sequence with at least about 70%, 75%, 80%, 85%, 90%, 91%, 92%, 93%, 94%, 95%, 96%, 97%, 98% or 99% identity to SEQ ID NO: 48.

In some embodiments, a CAR of the invention comprises an anti-CD20 scFv (e.g., comprising or consisting of the binding domain sequence of SEQ ID NO: 4, 5 or 6), linked to the amino acid sequence of SEQ ID NO: 48 or a sequence or an amino acid sequence with at least about 70%, 75%, 80%, 85%, 90%, 91%, 92%, 93%, 94%, 95%, 96%, 97%, 98% or 99% identity to SEQ ID NO: 48.

In some embodiments, a CAR of the invention comprises a sequence comprising a hinge region of human CD8, a transmembrane domain of human TNFR2, a costimulatory intracellular signaling domain of human TNFR2 and a primary intracellular signaling domain of human CD3ζ chain, wherein said sequence corresponds to the amino acid sequence of SEQ ID NO: 49, 50, 51, or 110 or an amino acid sequence with at least about 70%, 75%, 80%, 85%, 90%, 91%, 92%, 93%, 94%, 95%, 96%, 97%, 98% or 99% identity to SEQ ID NO: 49, 50, 51, or 110.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 49, 50, 51 or 110 or a sequence with at least about 70%, 75%, 80%, 85%, 90%, 91%, 92%, 93%, 94%, 95%, 96%, 97%, 98% or 99% identity to SEQ ID NO: 49, 50, 51 or 110.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 49, 50, 51 or 110 or a sequence with at least about 70%, 75%, 80%, 85%, 90%, 91%, 92%, 93%, 94%, 95%, 96%, 97%, 98% or 99% identity to SEQ ID NO: 49, 50, 51 or 110.

In some embodiments, a CAR of the invention comprises an anti-CD19 scFv (e.g., comprising or consisting of the binding domain sequence of SEQ ID NO: 1 or 2) linked to an amino acid sequence of SEQ ID NO: 49, 50, 51, or 110 or a sequence with at least about 70%, 75%, 80%, 85%, 90%, 91%, 92%, 93%, 94%, 95%, 96%, 97%, 98% or 99% identity to SEQ ID NO: 49, 50, 51, or 110.

In some embodiments, a CAR of the invention comprises an anti-CD20 scFv (e.g., comprising or consisting of the binding domain sequence of SEQ ID NO: 4, 5 or 6) linked to an amino acid sequence of SEQ ID NO: 49, 50, 51, or 110 or a sequence with at least about 70%, 75%, 80%, 85%, 90%, 91%, 92%, 93%, 94%, 95%, 96%, 97%, 98% or 99% identity to SEQ ID NO: 49, 50, 51, or 110.

In some embodiments, a CAR of the invention comprises a sequence comprising a hinge region of human CD8, a transmembrane domain of human TNFR2 and a primary intracellular signaling domain of human CD3ζ chain, wherein said sequence corresponds to the amino acid sequence of SEQ ID NO: 52 or an amino acid sequence with at least about 70%, 75%, 80%, 85%, 90%, 91%, 92%, 93%, 94%, 95%, 96%, 97%, 98% or 99% identity to SEQ ID NO: 52.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 52 or a sequence with at least about 70%, 75%, 80%, 85%, 90%, 91%, 92%, 93%, 94%, 95%, 96%, 97%, 98% or 99% identity to SEQ ID NO: 52.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 52 or a sequence with at least about 70%, 75%, 80%, 85%, 90%, 91%, 92%, 93%, 94%, 95%, 96%, 97%, 98% or 99% identity to SEQ ID NO: 52.

In some embodiments, a CAR of the invention comprises an anti-CD19 scFv (e.g., comprising or consisting of the binding domain sequence of SEQ ID NO: 1 or 2) linked to an amino acid sequence of SEQ ID NO: 52 or a sequence with at least about 70%, 75%, 80%, 85%, 90%, 91%, 92%, 93%, 94%, 95%, 96%, 97%, 98% or 99% identity to SEQ ID NO: 52.

In some embodiments, a CAR of the invention comprises an anti-CD20 scFv (e.g., comprising or consisting of the binding domain sequence of SEQ ID NO: 4, 5 or 6) linked to an amino acid sequence of SEQ ID NO: 52 or a sequence with at least about 70%, 75%, 80%, 85%, 90%, 91%, 92%, 93%, 94%, 95%, 96%, 97%, 98% or 99% identity to SEQ ID NO: 52.

In some embodiments, a CAR of the invention comprises a sequence comprising a hinge region of human CD8, a transmembrane domain of human CD8, a primary intracellular signaling domain of human CD3ζ chain and a costimulatory intracellular signaling domain of human TNFR2, wherein said sequence corresponds to the amino acid sequence of SEQ ID NO: 53 or an amino acid sequence with at least about 70%, 75%, 80%, 85%, 90%, 91%, 92%, 93%, 94%, 95%, 96%, 97%, 98% or 99% identity to SEQ ID NO: 53.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 53 or a sequence with at least about 70%, 75%, 80%, 85%, 90%, 91%, 92%, 93%, 94%, 95%, 96%, 97%, 98% or 99% identity to SEQ ID NO: 53.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 53 or a sequence with at least about 70%, 75%, 80%, 85%, 90%, 91%, 92%, 93%, 94%, 95%, 96%, 97%, 98% or 99% identity to SEQ ID NO: 53.

In some embodiments, a CAR of the invention comprises an anti-CD19 scFv (e.g., comprising or consisting of the binding domain sequence of SEQ ID NO: 1 or 2) linked to an amino acid sequence of SEQ ID NO: 53 or a sequence with at least about 70%, 75%, 80%, 85%, 90%, 91%, 92%, 93%, 94%, 95%, 96%, 97%, 98% or 99% identity to SEQ ID NO: 53.

In some embodiments, a CAR of the invention comprises an anti-CD20 scFv (e.g., comprising or consisting of the binding domain sequence of SEQ ID NO: 4, 5 or 6) linked to an amino acid sequence of SEQ ID NO: 53 or a sequence with at least about 70%, 75%, 80%, 85%, 90%, 91%, 92%, 93%, 94%, 95%, 96%, 97%, 98% or 99% identity to SEQ ID NO: 53.

In some embodiments, a CAR of the invention comprises a sequence comprising a hinge region of human CD8, a transmembrane domain of human CD8, a costimulatory intracellular signaling domain of human TNFR2 and a primary intracellular signaling domain of human CD3ζ chain, wherein said sequence corresponds to the amino acid sequence of SEQ ID NO: 54 or an amino acid sequence with at least about 70%, 75%, 80%, 85%, 90%, 91%, 92%, 93%, 94%, 95%, 96%, 97%, 98% or 99% identity to SEQ ID NO: 54.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 54 or a sequence with at least about 70%, 75%, 80%, 85%, 90%, 91%, 92%, 93%, 94%, 95%, 96%, 97%, 98% or 99% identity to SEQ ID NO: 54.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 54 or a sequence with at least about 70%, 75%, 80%, 85%, 90%, 91%, 92%, 93%, 94%, 95%, 96%, 97%, 98% or 99% identity to SEQ ID NO: 54.

In some embodiments, a CAR of the invention comprises an anti-CD19 scFv (e.g., comprising or consisting of the binding domain sequence of SEQ ID NO: 1 or 2) linked to an amino acid sequence of SEQ ID NO: 54 or a sequence with at least about 70%, 75%, 80%, 85%, 90%, 91%, 92%, 93%, 94%, 95%, 96%, 97%, 98% or 99% identity to SEQ ID NO: 54.

In some embodiments, ae CAR of the invention comprises an anti-CD20 scFv (e.g., comprising or consisting of the binding domain sequence of SEQ ID NO: 4, 5 or 6) linked to an amino acid sequence of SEQ ID NO: 54 or a sequence with at least about 70%, 75%, 80%, 85%, 90%, 91%, 92%, 93%, 94%, 95%, 96%, 97%, 98% or 99% identity to SEQ ID NO: 54.

In some embodiments, a CAR of the invention comprises a sequence comprising a hinge region of human CD8, a combination of human CD8 transmembrane domain and human TNFR2 transmembrane domain, a costimulatory intracellular signaling domain of human TNFR2 and a primary intracellular signaling domain of human CD3ζ chain, wherein said sequence corresponds to the amino acid sequence of SEQ ID NO: 55, 56 or 57 or an amino acid sequence with at least about 70%, 75%, 80%, 85%, 90%, 91%, 92%, 93%, 94%, 95%, 96%, 97%, 98% or 99% identity to SEQ ID NO: 55, 56 or 57.

In some embodiments, a CAR of the invention comprises an anti-HLA-A2 scFv (e.g., comprising or consisting of the binding domain sequence of SEQ ID NO: 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106 or 107), linked to the amino acid sequence of SEQ ID NO: 55, 56 or 57 or a sequence with at least about 70%, 75%, 80%, 85%, 90%, 91%, 92%, 93%, 94%, 95%, 96%, 97%, 98% or 99% identity to SEQ ID NO: 55, 56 or 57.

In some embodiments, a CAR of the invention comprises an anti-IL-23R scFv (e.g., comprising or consisting of the binding domain sequence of SEQ ID NO: 65, 66 or 67), linked to the amino acid sequence of SEQ ID NO: 55, 56 or 57 or a sequence with at least about 70%, 75%, 80%, 85%, 90%, 91%, 92%, 93%, 94%, 95%, 96%, 97%, 98% or 99% identity to SEQ ID NO: 55, 56 or 57.

In some embodiments, a CAR of the invention comprises an anti-CD19 scFv (e.g., comprising or consisting of the binding domain sequence of SEQ ID NO: 1 or 2) linked to an amino acid sequence of SEQ ID NO: 55, 56 or 57 or a sequence with at least about 70%, 75%, 80%, 85%, 90%, 91%, 92%, 93%, 94%, 95%, 96%, 97%, 98% or 99% identity to SEQ ID NO: 55, 56 or 57.

In some embodiments, a CAR of the invention comprises an anti-CD20 scFv (e.g., comprising or consisting of the binding domain sequence of SEQ ID NO: 4, 5 or 6) linked to an amino acid sequence of SEQ ID NO: 55, 56 or 57 or a sequence with at least about 70%, 75%, 80%, 85%, 90%, 91%, 92%, 93%, 94%, 95%, 96%, 97%, 98% or 99% identity to SEQ ID NO: 55, 56 or 57.

In some embodiments, a CAR of the invention (e.g., any CAR described herein comprising a TNFR2 transmembrane domain or a fragment or variant thereof and/or an intracellular domain comprising a TNFR2 costimulatory intracellular signaling domain or a fragment or variant thereof) has at least one of the following properties:
a) exhibits less expression at the cell surface than a CAR with the same sequence except that the transmembrane domain is a CD8 transmembrane domain and the costimulatory intracellular signaling domain is a 4-1BB costimulatory intracellular signaling domain;
b) exhibits comparable levels of CAR-specific activation as a CAR with the same sequence except that the transmembrane domain is a CD8 transmembrane domain and the costimulatory intracellular signaling domain is a 4-1BB costimulatory intracellular signaling domain;
c) does not reduce cell surface levels of a regulatory T cell marker such as any one or more (e.g., all) of FoxP3, Helios, and CD62L after at least 1, 2, 3, 4, 5, 6, 7, 8, 9, 10, 11, 12, 13, 14, 15, 16, 17, 18, 19, 20, or more days of culture; and
d) does not increase cell surface levels of a non-regulatory T cell marker such as CD127 after at least 1, 2, 3, 4, 5, 6, 7, 8, 9, 10, 11, 12, 13, 14, 15, 16, 17, 18, 19, 20, or more days of culture.

### Immune Cells

The present invention further relates to an immune cell expressing a CAR as described herein, and to a population of such immune cells.

In some embodiments, a nucleic acid encoding a CAR of the present invention is introduced into an immune cell, thereby generating an engineered cell expressing the CAR on the cell surface.

As used herein, the term “immune cells” generally includes white blood cells (leukocytes) that are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, dendritic cells).

In some embodiments, an immune cell of the invention is genetically modified to express a chimeric receptor comprising a TNFR2 transmembrane domain or a fragment or variant thereof and/or a TNFR2 costimulatory intracellular signaling domain or a fragment or variant thereof.

In some embodiments, an immune cell of the invention is a mammalian immune cell, e.g., a human immune cell, an immune cell from a farm animal (e.g., a cow, pig, or horse), or an immune cell from a pet (e.g., a cat or a dog).

In some embodiments, the immune cell is selected from the group consisting of lymphocytes, myeloid-derived cells, and any combination thereof. In certain embodiments, the immune cell is a lymphocyte, e.g., selected from the group consisting of T cells, B cells, natural killer (NK) cells, and any combination thereof. In particular embodiments, the immune cell is a T cell, which in certain embodiments is selected from the group consisting of CD4⁺ T cells, CD8⁺ T cells, γδ T cells, double negative (DN) T cells, and any combination thereof. In certain embodiments, the immune cell is a CD4⁺ T cell, such as, for example, a T helper cell, a regulatory T cell, an effector T cell, and any combination thereof. In some embodiments, the immune cell is a CD8⁺ T cell, such as, for example, a cytotoxic CD8⁺ T cell or a CD8⁺ regulatory T cell. In some embodiments, the immune cell is a γδ T cell. In some embodiments the immune cell is a T cell engineered to express a defined Gamma delta TCR (TEGγδ) cells. In some embodiments, the immune cell is a DN T cell. In some embodiments, the immune cell is a B cell. In some embodiments, the immune cell is a NK cell.

In some embodiments, the immune cell is a myeloid-derived cell, e.g., selected from the group consisting of neutrophils, eosinophils, basophils, monocytes, macrophages, dendritic cells, or any combination thereof. In certain embodiments, the immune cell is a macrophage. In certain embodiments, the immune cell is a dendritic cell.

In some embodiments, the immune cell is a regulatory immune cell, such as, for example, any regulatory immune cell suitable for use in cellular therapy. In certain embodiments, the regulatory immune cell is selected from the group consisting of a regulatory T cell, a CD4⁺ regulatory T cell, a CD8⁺ regulatory T cell, a regulatory γδ T cell, a regulatory DN T cell, a regulatory B cell, a regulatory NK cell, a regulatory macrophage, a regulatory dendritic cell, and any combination thereof.

In some embodiments, the regulatory immune cell is a regulatory T cell (Treg), in particular, a thymus derived Treg or an adaptive or induced Treg. In certain embodiments, the immune cell is a CD4⁺ regulatory T cell (Treg). In certain embodiments, the Treg is a thymus derived Treg or an adaptive or induced Treg. In certain embodiments, the Treg is a CD4⁺FoxP3⁺ regulatory T cell or a CD4⁺FoxP3⁻ regulatory T cell (Tr1 cell). In particular embodiments, the immune cell is a CD4⁺FoxP3⁺ regulatory T cell.

In some embodiments, the immune cell is a CD8⁺ regulatory T cell. Examples of CD8⁺ regulatory T cells include, but are not limited to, a CD8⁺CD28⁻ regulatory T cell, a CD8⁺CD103⁺ regulatory T cell, a CD8⁺FoxP3⁺ regulatory T cell, a CD8⁺CD122⁺ regulatory T cell, and any combination thereof.

In some embodiments, the regulatory immune cell is a regulatory γδ T cell.

In some embodiments, the regulatory immune cell is a regulatory DN T cell.

In some embodiments, the regulatory immune cell is a regulatory B cell. An example of a regulatory B cell includes, but is not limited to, a CD19⁺CD24^{hi}CD38^{hi} B cell.

In some embodiments, the regulatory immune cell is a regulatory NK cell.

In some embodiments, the regulatory immune cell is a regulatory macrophage.

In some embodiments, the regulatory immune cell is a regulatory dendritic cell.

In some embodiments, the immune cell is an effector immune cell, such as, for example, any effector immune cell suitable for use in cellular therapy. In certain embodiments, the effector immune cell is selected from the group consisting of an effector T cell, a CD4⁺ effector T cell, a CD8⁺ effector T cell, an effector γδ T cell, an effector DN T cell, an effector B cell, an effector NK cell, an effector macrophage, an effector dendritic cell, and any combination thereof.

In some embodiments, the immune cell is an effector T cell. In certain embodiments, the effector immune cell is a CD4⁺ effector T cell. Examples of CD4⁺ effector T cells include, but are not limited to, Th1 cells, Th2 cells, Th9 cells, Th17 cells, Th22 cells, CD4⁺T follicular helper (Tfh) cells, and any combination thereof. In some embodiments, the effector immune cell is a CD8⁺ effector T cell. Examples of CD8⁺ effector T cells include, but are not limited to, a CD8⁺CD45RO⁺CCR7⁻CD62L⁻ effector T cell, a CD8⁺CD45RA⁺CCR7⁻CD62L⁻ effector T cell, and any combination thereof.

In one embodiment, the immune cell is an effector γδ T cell.

In one embodiment, the immune cell is an effector DN T cell.

In one embodiment, the immune cell is an effector B cell. Examples of effector B cells include, but are not limited to, a CD19⁺CD25^{hi} B cell, activated B cells and plasma cells, and any combination thereof.

In some embodiments, the immune cell is an effector NK cell.

In some embodiments, the immune cell is an effector macrophage.

In some embodiments, the immune cell is an effector dendritic cell.

In some embodiments, the immune cell is selected from the group consisting of T cells, natural killer (NK) cells, γδ T cells, double negative (DN) cells, regulatory immune cells, regulatory T cells, effector immune cells, effector T cells, B cells and myeloid-derived cells, and any combination thereof.

In some embodiments, a nucleic acid encoding a CAR of the present invention is introduced into a pluripotent stem cell (PSC), which may then be differentiated to a T cell. PSCs are cells capable to giving rise to any cell type in the body and include, for example, embryonic stem cells (ESCs), PSCs derived by somatic cell nuclear transfer, and induced PSCs (iPSCs). As used herein, the term “embryonic stem cells” refers to pluripotent stem cells obtained from early embryos; in some embodiments, this term refers to ESCs obtained from a previously established embryonic stem cell line and excludes stem cells obtained by recent destruction of a human embryo.

In some embodiments, a nucleic acid encoding a CAR of the present invention is introduced into a multipotent cell such as a hematopoietic stem cell (HSCs such as those isolated from bone marrow or cord blood), hematopoietic progenitor cells (e.g., lymphoid progenitor cell), or mesenchymal stem cells (MSC). Multipotent cells are capable of developing into more than one cell type, but are more limited than cell type potential than pluripotent cells. The multipotent cells may be derived from established cell lines or isolated from human bone marrow or umbilical cords. By way of example, the HSCs may be isolated from a patient or a healthy donor following G-CSF-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45 (B cells), GR-1 (granulocytes), and Iad (differentiated antigen-presenting cells). HSCs can then be positively selected by antibodies to CD34.

In some embodiments, a nucleic acid encoding a CAR of the present invention is introduced into a non-Treg lymphoid cell that is differentiated into a Treg cell after genome editing. The edited non-Treg cells may be differentiated into Treg cells before engrafting into a patient as described above. Alternatively, the edited non-Treg cells may be induced to differentiate into Treg cells after engrafting into a patient.

In some embodiments, the expression level of molecules is determined by flow cytometry, immunofluorescence or image analysis, for example high content analysis. In certain embodiments, the expression level of molecules is determined by flow cytometry. In particular embodiments, before conducting flow cytometry analysis, cells are fixed and permeabilized, thereby allowing detection of intracellular proteins.

In some embodiments, determining the expression level of a molecule in a cell population comprises determining the percentage of cells of the cell population expressing the molecule (i.e., cells “+” for the molecule). In certain embodiments, said percentage of cells expressing the molecule is measured by FACS.

The terms “expressing,” “positive,” or “+” and “not expressing,” “negative,” or −” are well known in the art and refer to the expression level of the cell marker of interest, in that the expression level of the cell marker corresponding to “+” is high or intermediate (also referred to as “+/−”), and the expression level of the cell marker corresponding to “−” is null.

The term “low” or “lo” or “lo/−” is well known in the art and refers to the expression level of the cell marker of interest, in that the expression level of the cell marker is low in comparison with the expression level of that cell marker in the population of cells being analyzed as a whole. More particularly, the term “lo” refers to a distinct population of cells that express the cell marker at a lower level than one or more other distinct populations of cells.

The term “high” or “hi” or “bright” is well known in the art and refers to the expression level of the cell marker of interest, in that the expression level of the cell marker is high in comparison with the expression level of that cell marker in the population of cells being analyzed as a whole.

Generally, cells in the top 2, 3, 4, or 5% of staining intensity are designated “hi,” with those falling in the top half of the population categorized as being “+”. Those cells falling below 50%, of fluorescence intensity are designated as “lo” cells and below 5% as “−” cells.

The expression level of the cell marker of interest is determined by comparing the Median Fluorescence Intensity or Mean Fluorescence Intensity (MFI) of the cells from the cell population stained with fluorescently labeled antibody specific for this marker to the fluorescence intensity (FI) of cells from the same cell population stained with fluorescently labeled antibody with an irrelevant specificity but with the same isotype, the same fluorescent probe and originated from the same species (referred to as isotype control). The cells from the population stained with fluorescently labeled antibody specific for this marker and that show equivalent MFI or a lower MFI than the cells stained with the isotype control are considered as not expressing this marker and are designated (−) or negative. The cells from the population stained with fluorescently labeled antibody specific for this marker and that show a MFI value superior to the cells stained with the isotype control are considered as expressing this marker and are designated (+) or positive.

The invention also relates to an isolated and/or substantially purified population of immune cells as defined herein.

Thus, the invention provides an isolated and/or substantially purified population of immune cells, wherein the cells of the population comprise a CAR as described herein, e.g., a CAR that comprises a TNFR2 transmembrane domain or a fragment or variant thereof and/or a TNFR2 costimulatory intracellular signaling domain or a fragment or variant thereof.

As used herein, an “isolated population” refers to a cell population that is removed from its natural environment (such as the peripheral blood) and that is isolated, purified or separated, and is at least about 75% free, 80% free, 85% free, and in certain embodiments about 90%, 95%, 96%, 97%, 98%, 99% free, from other cells with which it is naturally present, but which lack the cell surface markers based on which the cells were isolated.

The present invention further relates to an enriched population of immune cells as defined herein.

In some embodiments, the isolated, purified and/or enriched immune cell population of the invention has been frozen and thawed.

In some embodiments, the T cells of the immune cell population of the invention express a chimeric receptor (CAR) as described herein and may thus be defined as CAR-monospecific (i.e., all the Treg cells recognize the same antigen with the CAR they express).

In some embodiments, the Treg cell population is TCR-monospecific (i.e., all the Treg cells recognize the same antigen with their TCR). In some embodiments, the Treg cell population is TCR-polyspecific (i.e., the Treg cells may recognize different antigens with their TCR).

In some embodiments, the T cell population is TCR-monospecific, and the TCR recognizes an antigen, a fragment of an antigen, a variant of an antigen or a mixture thereof.

In some embodiments, the T cell population is TCR-monospecific, and the TCR is specific for a food antigen from the common human diet.

In some embodiments, the T cell population is TCR-monospecific, and the TCR is specific for an autoantigen, such as, for example, a multiple sclerosis-associated antigen, a joint-associated antigen, an eye-associated antigen, a human HSP antigen, a skin-associated antigen or an antigen involved in graft rejection or GVHD. Examples of such autoantigens are given herein.

In some embodiments, the T cell population is TCR-monospecific, and the TCR is specific for an inhaled allergen, an ingested allergen or a contact allergen.

In some embodiments, the T cell population is TCR-monospecific, and the TCR is specific for an antigen selected from the group consisting of ovalbumin, MOG, type II collagen, citrullinated vimentin, citrullinated type II collagen, citrullinated fibrinogen, and fragments, variants and mixtures thereof.

In some embodiments, the T cell population is TCR-monospecific, and the TCR is specific for ovalbumin or a fragment, variant, or mixture thereof.

In some embodiments, the T cell population is TCR-monospecific, and the TCR is specific for MOG or a fragment, variant, or mixture thereof.

In some embodiments, the T cell population is TCR-monospecific, and the TCR is specific for type II collagen or a fragment, variant, or mixture thereof.

In some embodiments, the T cell population is TCR-monospecific, and the TCR is specific for citrullinated vimentin, citrullinated type II collagen, citrullinated fibrinogen, or a fragment, variant, or mixture thereof.

In some embodiments, the T cell population is TCR-monospecific, and the TCR is specific for HLA-A2 or a fragment, variant, or mixture thereof (e.g., as described herein).

In some embodiments, the T cell population is TCR-monospecific, and the TCR is specific for IL-23R or a fragment, variant, or mixture thereof (e.g., as described herein).

In some embodiments, the T cell population is TCR-monospecific, and the TCR is specific for a B cell surface marker, such as, for example, CD19 or CD20 or a fragment, variant, or mixture thereof (e.g., as described herein).

In some embodiments, the T cell population is TCR-monospecific, and the TCR is specific for a cancer antigen or a fragment, variant, or mixture thereof, as described herein.

In some embodiments, the T cell population is TCR-monospecific, and the TCR recognizes infected cells. In some embodiments, the T cell population is TCR-monospecific, and the TCR is specific for a bacterial antigen or a fragment, variant, or mixture thereof. In some embodiments, the T cell population is TCR-monospecific, and the TCR is specific for a viral antigen or a fragment, variant, or mixture thereof. In some embodiments, the T cell population is TCR-monospecific, and the TCR is specific for a fungal antigen or a fragment, variant, or mixture thereof. In some embodiments, the T cell population is TCR-monospecific, and the TCR is specific for a parasitic antigen or a fragment, variant, or mixture thereof.

In some embodiments, immune cells expressing a CAR of the invention are suppressive against cells recognized by the CAR. In certain embodiments, the immune cells are T cells. In particular embodiments, the immune cells are Treg cells. In some embodiments, the Treg cells are obtained by in vitro differentiation of naïve T cells.

In some embodiments, immune cells of the invention are not cytotoxic. In certain embodiments, the immune cells are T cells. In particular embodiments, the immune cells are Treg cells.

In some embodiments, regulatory immune cells of the invention may be selected form the group consisting of CD4⁺CD25⁺FoxP3⁺ Treg, Tr1 cells, TGF-β secreting Th3 cells, regulatory NK T cells, regulatory γδ T cells, regulatory CD8⁺ T cells, and double negative regulatory T cells.

In some embodiments, the immune cells of the invention are cytotoxic. In certain embodiments, the immune cells are cytotoxic for cells expressing a cancer antigen or a fragment or variant thereof, as described herein. In particular embodiments, the immune cells of the invention are cytotoxic for cancerous cells. In particular embodiments, the immune cells of the invention are cytotoxic for infected cells.

In some embodiments, the immune cells of the invention are cytotoxic immune cells. Cytotoxic immune cells comprise, for example, cytotoxic T cells, CD8⁺ T cells, natural killer (NK) cells, cytotoxic B cells, macrophages, and monocytes. Upon activation, each of these cytotoxic immune cells triggers the destruction of target cells. For example, cytotoxic immune cells trigger the destruction of target cancer cells by either or both of the following means. First, upon activation, immune cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via FAS-FAS ligand interaction between the immune cells and target tumor cells.

In some embodiments, the cytotoxic immune cells are autologous cells.

In some embodiments, the cytotoxic immune cells are heterologous cells.

In some embodiments, the cytotoxic immune cells are allogenic cells.

In some embodiments, the immune cells of the invention are cytotoxic for proB cells, preB cells, immature (or transitional) B cells, mature naïve B cells, activated B cells, memory B cells, plasma cells, lymphoblasts, marginal zone B cells, germinal center B cells, plasmablasts and/or regulatory B cells (Breg cells).

In some embodiments, the immune cell of the invention is not cytotoxic for Breg cells.

In some embodiments, the monospecific Treg cell population of the invention is cytotoxic for B cells expressing and presenting an immunoglobulin at their surface. Examples of B cells expressing and presenting an immunoglobulin at their surface include, but are not limited to, preB cells, immature (or transitional) B cells, mature naïve B cells, activated B cells, memory B cells, marginal zone B cells, germinal center B cells, and regulatory B cells (Breg cells).

In some embodiments, the monospecific Treg cell population of the invention is cytotoxic for mature B cells, e.g., for mature activated B cells. In certain embodiments, the monospecific Treg cell population of the invention is cytotoxic for mature B cells (e.g., mature activated B cells) expressing at their surface a B cell surface marker recognized by the chimeric receptor expressed by the cells of the monospecific Treg cell population.

In some embodiments, the monospecific Treg cell population of the invention is cytotoxic for at least one cell type selected from proB cells, preB cells, immature (or transitional) B cells, mature naïve B cells, activated B cells, memory B cells, plasma cells, lymphoblasts, marginal zone B cells, germinal center B cells, plasmablasts and regulatory B cells (Breg cells). In particular embodiments, the monospecific Treg cell population is cytotoxic for at least one cell type selected from preB cells, immature (or transitional) B cells, mature naïve B cells, activated B cells, memory B cells, marginal zone B cells, germinal center B cells, and regulatory B cells (Breg cells).

In some embodiments, the monospecific Treg cell population of the invention is cytotoxic for at least one cell type selected from proB cells, preB cells, immature (or transitional) B cells, mature naïve B cells, activated B cells, memory B cells, plasma cells, lymphoblasts, marginal zone B cells, germinal center B cells, and plasmablasts. In particular embodiments, the monospecific Treg cell population is cytotoxic for at least one cell type selected from preB cells, immature (or transitional) B cells, mature naïve B cells, activated B cells, memory B cells, marginal zone B cells, and germinal center B cells.

In some embodiments, an immune cell population of the invention expresses at its cell surface a CAR of the invention (herein referred to as “first receptor”), and another receptor (herein referred to as “second receptor”) that binds to another, distinct ligand. In certain embodiments, the second receptor comprises an extracellular ligand binding domain, optionally a hinge domain, at least one transmembrane domain, and at least one intracellular signaling domain, e.g., as described herein.

In some embodiments, the second receptor is endogenous (such as, for example, the endogenous TCR). In some embodiments, the second receptor is exogenous, and its expression is induced in the immune cell population of the invention by transformation or transduction of a nucleic acid encoding it. Said exogenous receptor may be an exogenous TCR or a CAR. Therefore, in some embodiments, the immune cell population of the invention expresses two CARs, wherein the first one and the second one each recognize a distinct ligand. In some embodiments, the immune cell population of the invention expresses two CARs, wherein the first one recognizes a first epitope on an antigen, and the second one recognizes a second, distinct epitope on the same antigen. In some embodiments, the immune cell population of the invention expresses two CARs, wherein the first one recognizes an antigen, and the second one recognizes a second, distinct antigen (such as, for example, an antigen variant).

In some embodiments, at least one of the CAR of the invention and the second receptor (e.g., a second CAR) is inducible, i.e., its expression on the cell surface may be induced.

In some embodiments, the expression of at least one of the CAR of the invention and the second receptor (e.g., second CAR) is induced by the activation of the other receptor. In certain embodiments, the expression of the CAR of the invention is induced by the activation of the second receptor. In certain embodiments, the expression of the second receptor is induced by the activation of the CAR of the invention. Inducible CARs have been described in the art, such as, for example, by Roybal et al (Cell 167(2):419-432 (2016)).

In some embodiments, the second receptor (e.g., a second CAR) is specific for an antigen, a fragment of an antigen, a variant of an antigen or a mixture thereof.

In some embodiments, the second receptor (e.g., a second CAR) is specific for a food antigen from the common human diet.

In some embodiments, the second receptor (e.g., a second CAR) is specific for an autoantigen (e.g., an autoantigen described herein), such as, for example, a multiple sclerosis-associated antigen, a joint-associated antigen, an eye-associated antigen, a human HSP antigen, a skin-associated antigen or an antigen involved in graft rejection or GVHD. In certain embodiments, the antigen is a skin-associated antigen. In certain embodiments, the antigen is an antigen involved in graft rejection or GVHD.

In some embodiments, the second receptor (e.g., a second CAR) is specific for an inhaled allergen, an ingested allergen or a contact allergen.

In some embodiments, the second receptor (e.g., a second CAR) is specific for an antigen selected from the group consisting of ovalbumin, MOG, type II collagen fragments, citrullinated vimentin, citrullinated type II collagen, citrullinated fibrinogen, and fragments, variants and mixtures thereof.

In some embodiments, the second receptor (e.g., a second CAR) is specific for ovalbumin or a fragment, variant or mixture thereof.

In some embodiments, the second receptor (e.g., a second CAR) is specific for MOG or a fragment, variant or mixture thereof.

In some embodiments, the second receptor (e.g., a second CAR) is specific for type II collagen or a fragment, variant or mixture thereof.

In some embodiments, the second receptor (e.g., a second CAR) is specific for citrullinated vimentin, citrullinated type II collagen, citrullinated fibrinogen or a fragment, variant or mixture thereof.

In some embodiments, the second receptor (e.g., a second CAR) is specific for HLA-A2 or a fragment, variant or mixture thereof.

In some embodiments, the second receptor (e.g., a second CAR) is specific for IL-23R or a fragment, variant or mixture thereof.

In some embodiments, the second receptor (e.g., a second CAR) is specific for a B cell surface marker, such as, for example, CD19 or CD20, or a fragment, variant or mixture thereof.

In some embodiments, the second receptor (e.g., a second CAR) is specific for a cancer antigen or a fragment, variant or mixture thereof, as described herein.

In some embodiments, the second receptor (e.g., a second CAR) recognizes infected cells. In some embodiments, the second receptor (e.g., a second CAR) is specific for a bacterial antigen or a fragment, variant or mixture thereof. In some embodiments, the second receptor (e.g., a second CAR) is specific for a viral antigen or a fragment, variant or mixture thereof. In some embodiments, the second receptor (e.g., a second CAR) is specific for a fungal antigen or a fragment, variant or mixture thereof.

In some embodiments, the extracellular binding domain of the second receptor is a protein or a fragment or a variant thereof, such as for example, an autoantigen or a fragment or variant thereof.

In some embodiments, the second receptor (e.g., a second CAR) is specific for an autoantibody, such as for example, an autoantibody expressed on a B cell or a fragment, variant or mixture thereof.

In some embodiments, a CAR of the invention comprises a first intracellular signaling domain, and the second receptor comprises a distinct second intracellular signaling domain. In certain embodiments, a CAR of the invention comprises a T cell primary signaling domain (such as, for example, CD3 zeta), and the second receptor comprises a costimulatory signaling domain (such as, for example, the costimulatory domain of TNFR2 or CD8 or a combination costimulatory signaling domain of TNFR2 and CD8). In certain embodiments, a CAR of the invention comprises a costimulatory intracellular signaling domain (such as, for example, the costimulatory intracellular signaling domain of TNFR2, 4-1BB, CD27, or CD28 or a combination costimulatory intracellular signaling domain of TNFR2 and 4-1BB), and the second receptor comprises a T cell primary intracellular signaling domain (such as, for example, CD3 zeta). According to these embodiments, the complete activation of the immune cell population of the invention requires both the binding of the CAR of the invention to the ligand to which it is directed, and the binding of the second receptor to the ligand to which it is directed.

In some embodiments, the ligand recognized by the second receptor is expressed or present at the diseased tissue or organ, or at the site of the autoimmune response. Consequently, suppressive activity for cells expressing the ligand of the first CAR of the invention will be induced only at the diseased tissue or organ or at the site of the autoimmune response, when said ligand will be present and recognized by the second receptor on the cells of the immune cell (e.g., Treg) population.

In some embodiments, the second chimeric receptor further comprises an extracellular ligand binding domain recognizing a ligand distinct from the ligand recognized by the first chimeric receptor of the invention. In some embodiments, said ligand binding domain is an antibody or an antigen binding fragment thereof. In some embodiments, the second chimeric receptor further comprises an extracellular ligand binding domain recognizing a distinct epitope of the same antigen recognized by the first chimeric receptor.

In some embodiments, the chimeric receptor of the invention comprises an extracellular ligand binding domain comprising a first ligand binding domain that binds to a first ligand and a second ligand binding domain that binds to a second ligand distinct from said first ligand. In some embodiments, said ligand binding domain is a bifunctional antibody recognizing both the first and the second ligand. In some embodiments, said ligand binding domain is a bifunctional antibody recognizing two distinct epitopes of the same antigen.

### Nucleic Acids, Vectors, and CAR Preparation

The present invention also relates to a nucleic acid sequence encoding a CAR as described herein, wherein said nucleic acid sequence comprises:
at least one nucleic acid sequence of an extracellular binding domain,
optionally at least one nucleic acid sequence of an extracellular hinge domain, at least one nucleic acid sequence of a transmembrane domain,
at least one nucleic acid sequence of an intracellular domain, wherein the at least one nucleic acid sequence of the intracellular domain comprises at least one nucleic acid sequence of a primary intracellular signaling domain and optionally at least one nucleic acid sequence of a costimulatory intracellular signaling domain,
   wherein the nucleic acid sequence of the transmembrane domain is a nucleic acid sequence of a human TNFR2 transmembrane domain or a fragment or variant thereof or a nucleic acid sequence of any transmembrane domain or a fragment or variant thereof or a combination thereof, and/or the nucleic acid sequence of the costimulatory intracellular signaling domain is a nucleic acid sequence of a human TNFR2 costimulatory intracellular signaling domain or a fragment or variant thereof or a nucleic acid sequence of any costimulatory intracellular signaling domain or a fragment or variant thereof or a combination thereof, wherein the nucleic acid sequence of the transmembrane domain is a nucleic acid sequence of a TNFR2 transmembrane domain or a fragment or variant thereof and/or the nucleic acid sequence of the costimulatory intracellular signaling domain is a TNFR2 costimulatory intracellular signaling domain or a fragment or variant thereof.

The invention also provides a vector comprising a CAR-encoding nucleic acid sequence as described herein. Examples of vectors that may be used in the present invention include, but are not limited to, a DNA vector, a RNA vector, a plasmid, a phagemid, a phage derivative, a virus and a cosmid.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses that are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (see, e.g., PCT Patent Publications WO 01/96584 and WO01/29058 and U.S. Pat. No. 6,326,193, incorporated herein by reference).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. Further, a number of adenovirus vectors are known in the art. In some embodiments, lentivirus vectors are used.

Additional transcriptionally active elements, e.g., promoters and enhancers, regulate the frequency of transcriptional initiation. Typically core promoter, these are located in the region 30-110 bp upstream of the start site, although a number of promoters have recently been shown to contain functional elements downstream of the start site as well, and enhancer elements are generally located 500-2000 bp upstream of the start site. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-la (EF-la). Another example of a suitable promoter is phosphoglycerate kinase (PGK) promoter. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence that it is operatively linked to when such expression is desired, or turning off the expression when expression is not desired. Examples of inducible promoters include, but are not limited to, a metallothionine promoter, a glucocorticoid promoter, a progesterone promoter, and a tetracycline promoter. In addition, bi-directional promoters allowing efficient and coordinated expression of two or more genes may also be of interest in the present invention. Examples of bi-directional promoters include, but are not limited to, the promoters described by Luigi Naldini in U.S. Patent Publication 2006/200869, incorporated herein by reference, disclosing a bi-directional promoter comprising i) a first minimal promoter sequence derived from cytomegalovirus (CMV) or mouse mammary tumor virus (MMTV) genomes and ii) a full efficient promoter sequence derived from an animal gene.

In order to assess the expression of a CAR polypeptide or portions thereof, the expression vector to be introduced into a T cell can also contain either a selectable marker gene such as CD34, CD271 or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in host cells. Useful selectable markers include, for example, antibiotic-resistance genes, such as neomycin and the like.

In some embodiments of the invention, suicide gene technology may be used. Different suicide gene technologies are described in the art depending on their mechanism of action (see, e.g., Jones et al., Frontiers in Pharmacology 5:254 (2014)). Examples of gene-directed enzyme prodrug therapy (GDEPT) converting a nontoxic drug to a toxic drug include herpes simplex virus thymidine kinase (HSV-TK) and cytosine deaminase (CD). Other examples are chimeric proteins composed of a drug binding domain linked to apoptotic components such as for example the inducible Fas (iFas) or the inducible Caspase 9 (iCasp9) systems. Other examples include systems mediated by therapeutic antibodies such as inducing overexpression of c-myc at the surface of the engineered cell to induce its deletion by administration of an anti-c-myc antibody. The use of EGFR is described as a similar system compared to the c-myc system.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (see, e.g., Ui-Tei et al., FEBS Letters 479:79-82 (2000)). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., a mammalian, bacterial, yeast, or insect cell, by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, of the invention, a polynucleotide is introduced into a host cell using calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-10 (1991)). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances that may be naturally occurring or synthetic lipids. For example, lipids include the fatty droplets that naturally occur in the cytoplasm as well as the class of compounds that contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; and dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, the immune cells of the invention are modified through the introduction of RNA. In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In certain embodiments, the template for in vitro transcription is the CAR of the present invention.

In some embodiments, the DNA to be used for PCR contains an open reading frame. The DNA may be, e.g., from a naturally occurring DNA sequence from the genome of an organism. In some embodiments, the DNA is a full-length gene of interest or a portion of a gene. The gene can include some or all of the 5′ and/or 3′ untranslated regions (UTRs). The gene can include exons and introns. In some embodiments, the DNA to be used for PCR is a human gene. In some embodiments, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR may be used to generate a template for in vitro transcription of mRNA that is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), which may include 5′ and 3′ UTRs. The primers can also be designed to amplify a portion of a gene that encodes a particular domain of interest. In some embodiments, the primers are designed to amplify the coding region of a human cDNA, including all or portions of the 5′ and 3′ UTRs. Primers useful for PCR are generated by synthetic methods that are well known in the art.

“Forward primers” are primers that contain a region of nucleotides that are substantially complementary to nucleotides on the DNA template that are upstream of the DNA sequence that is to be amplified. “Upstream” is used herein to refer to a location 5′ to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. In some embodiments, the RNA may have 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA. The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but do not appear to be required for all RNAs to enable efficient translation. The requirement for Kozak sequences for many mRNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail that determine ribosome binding, initiation of translation and stability of the mRNA in the cell. On a circular DNA template, for instance, plasmid DNA, RNA polymerase produces a long concatemeric product that is not suitable for expression in eukaryotic cells. The transcription of plasmid DNA linearized at the end of the 3′ UTR results in normal sized mRNA that is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequences integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method that allows construction of DNA templates with polyA/T 3′ stretch without cloning is highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines.

Poly(A) tails of RNAs can be further extended following in vitro transcription with the use of a poly(A) polymerase, such as E. coli polyA polymerase (E-PAP). In some embodiments, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on RNAs may also provide stability to RNA molecules. In some embodiments, RNAs produced by the methods disclosed herein include a 5′ cap. The 5′ cap is provided using techniques known in the art and described herein (Cougot et al., Trends in Biochem. Sci. 29:436-444 (2001); Stepinski et al., RNA 7:1468-95 (2001); Elango, et al., Biochim. Biophys. Res. Commun. 330:958-966 (2005)).

The RNAs produced by the methods disclosed herein can also contain an internal ribosome entry site (IRES) sequence. The IRES sequence may be any viral, chromosomal or artificially designed sequence that initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants, can be included.

RNA can be introduced into target cells using any of a number of different methods, for instance, commercially available methods which include, but are not limited to, electroporation (e.g., Amaxa Nucleofector-II (Amaxa Biosystems, Cologne, Germany), ECM 830 (BTX) (Harvard Instruments, Boston, Mass.), Gene Pulser II (BioRad, Denver, Colo.), or Multiporator (Eppendort, Hamburg Germany)), cationic liposome mediated transfection using lipofection, polymer encapsulation, peptide mediated transfection, or biolistic particle delivery systems such as “gene guns” (see, for example, Nishikawa et al. Hum Gene Ther. 12(8):861-70 (2001)).

In some embodiments, CAR sequences described herein are delivered into immune cells of the invention by using a retroviral or lentiviral vector. CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In some embodiments, the CAR sequences are delivered into immune cells of the invention by using in vitro transcribed mRNA. In vitro transcribed mRNA CARs can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In some embodiments, the desired CAR can be expressed in the cells by way of transposons.

In some embodiments, an immune cell of the invention is, e.g., a T cell. Prior to expansion and genetic modification of T cells (such as Treg cells) as described herein, the cells are obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In certain embodiments of the present invention, T cells can be obtained from a unit of blood collected from a subject using any number of techniques known to the skilled artisan, such as Ficoll™ separation, centrifugation through a PERCOLL™ gradient following red blood cell lysis and monocyte depletion, counterflow centrifugal elutriation, leukapheresis, and subsequent cell surface marker-based magnetic or flow cytometric isolation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, cells from the circulating blood of an individual are obtained by leukapheresis.

In some embodiments, cells collected by leukapheresis may be washed to remove the plasma fraction and to place the cells in an appropriate buffer or media for subsequent processing steps. In some embodiments of the invention, the cells are washed with phosphate buffered saline (PBS). In some embodiments, the wash solution lacks calcium and may lack magnesium or may lack many if not all divalent cations. After washing, the cells may be resuspended in any of a variety of biocompatible buffers, such as, for example, Ca²⁺-free, Mg²⁺-free PBS, PlasmaLyte A, or other saline solutions with or without buffer.

Alternatively, the undesirable components of the leukapheresis sample may be removed and the cells directly resuspended in culture media.

A specific subpopulation of T cells can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values therebetween. In a further embodiment, the time period is at least 1, 2, 3, 4, 5, or 6 hours. In some embodiments, the time period is 10 to 24 hours. In certain embodiments, the incubation time period is 24 hours. Longer incubation times may be used to isolate T cells in any situation where there are few T cells as compared to other cell types. Thus, by simply shortening or lengthening the time that T cells are allowed to bind to the anti-CD3/anti-CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention.

In some embodiments, it may be desirable to perform the selection procedure and use the “unselected” cells in the activation and expansion process. “Unselected” cells can also be subjected to further rounds of selection. Enrichment of a T cell population by negative selection can be accomplished with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in some embodiments, a concentration of 2 billion cells/mL is used. In some embodiments, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In some embodiments, a concentration of cells of 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and may be desirable to obtain.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation.

Also contemplated in the context of the invention is the collection of blood samples or leukapheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In some embodiments, a blood sample or a leukapheresis product is taken from a generally healthy subject. In certain embodiments, a blood sample or a leukapheresis product is taken from a generally healthy subject who is at risk of developing a disease, but who has not yet developed a disease, and the cells of interest are isolated and frozen for later use. In certain embodiments, the T cells may be expanded, frozen, and used at a later time.

Whether prior to or after genetic modification of the Treg cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041; and U.S. Patent Publication 2006/0121005, incorporated herein by reference.

Generally, the T cells (e.g., Treg cells) of the invention are expanded by contact with a surface having attached thereto an agent that stimulates a CD3/TCR complex associated signal and a ligand that stimulates a co-stimulatory molecule on the surface of the cells. In particular, the T cells (e.g., Treg cells) may be stimulated as described herein, such as by contact with an anti-CD3 antibody or antigen-binding fragment thereof or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of CD4⁺ T cells, an anti-CD3 antibody and an anti-CD28 antibody may be used. Examples of an anti-CD28 antibody include, without being limited to, 9.3, B-T3, XR-CD28 (Diaclone, Besancon, France). Other expansion methods commonly known in the art can be used (Berg et al., Transplant Proc. 30(8):3975-3977 (1998); Haanen et al., J. Exp. Med. 190(9):1319-1328 (1999); Garland et al., J. Immunol Meth. 227(1-2):53-63 (1999)).

In certain embodiments, the primary stimulatory signal and the co-stimulatory signal for the T cells (e.g., Tregs) of the invention may be provided by different protocols. For example, the agents providing each signal may be in solution and/or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In some embodiments, the agents may be in soluble form, and then cross-linked to a surface, such as a cell expressing Fc receptors or an antibody or other binding agent that will bind to the agents. In this regard, see for example, U.S. Patent Publications 2004/0101519 and 2006/0034810, incorporated herein by reference, for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4⁺ T cell expansion and T cell growth is used. In certain aspects of the present invention, a ratio of anti CD3:anti-CD28 antibodies bound to the beads is used such that an increase in T cell expansion is observed as compared to the expansion observed using a ratio of 1:1. In some embodiments, an increase of from about 1 to about 3 fold is observed as compared to the expansion observed using a ratio of 1:1. In some embodiments, the ratio of anti-CD3:anti-CD28 antibody bound to the beads ranges from 100:1 to 1:100 and all integer values therebetween. In some embodiments, more anti-CD28 antibody is bound to the particles than anti-CD3 antibody, i.e., the ratio of CD3:CD28 is less than one. In certain embodiments of the invention, the ratio of anti-CD28 antibody to anti CD3 antibody bound to the beads is greater than 2:1. In one particular embodiment, a 1:100 CD3:CD28 ratio of antibody bound to beads is used. In some embodiments, a 1:75 CD3:CD28 ratio of antibody bound to beads is used. In a further embodiment, a 1:50 CD3:CD28 ratio of antibody bound to beads is used. In some embodiments, a 1:30 CD3:CD28 ratio of antibody bound to beads is used. In certain embodiments, a 1:10 CD3:CD28 ratio of antibody bound to beads is used. In some embodiments, a 1:3 CD3:CD28 ratio of antibody bound to the beads is used. In some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads may only bind a few cells, while larger beads may bind many. In certain embodiments, T cells can be stimulated with a ratio of cells to particles ranging from 1:100 to 100:1 and any integer values in between. In particular embodiments, the ratio comprises 1:9 to 9:1 and any integer values in between. The ratio of anti-CD3- and anti-CD28-coupled particles to T cells that result in T cell stimulation can vary as noted above; however certain embodiments of values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one particular ratio being at least 1:1 particles per T cell. In some embodiments, a ratio of particles to cells of 1:1 or less is used. In certain embodiments, the particle: cell ratio is 1:5. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the immune cells are combined with agent-coated beads, the beads and the cells are subsequently separated, and then the cells are cultured. In alternative embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 antibodies are attached (3×28 beads) to contact the Treg cells of the invention. In some embodiments, the cells (for example, 10⁴ to 10⁹ T cells) and beads (for example, DYNABEADS® M-450 CD3/CD28 T paramagnetic beads at a ratio of 1:1) are combined in a buffer such as PBS (e.g., without divalent cations such as calcium and magnesium). Again, those of ordinary skill in the art can readily appreciate that any cell concentration may be used depending on the circumstance. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and/or cell expansion. Further, use of high cell concentrations allows more efficient capture of cells that may weakly express target antigens of interest, such as CD28-negative T cells. Such populations of cells may have therapeutic value and may be desirable to obtain in certain embodiments.

In some embodiments of the present invention, the mixture may be cultured for several hours (e.g., about 3 hours) to about 14 days or any hourly integer value in between. In some embodiments, the mixture may be cultured for 21 days. In some embodiments of the invention, the beads and the T cells are cultured together for about eight days. In some embodiments, the beads and T cells are cultured together for 2-3 days. Several cycles of stimulation may also be desired such that culture time of the T cells may be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (Th, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of Th cells, while after about days 8-9, the population of T cells comprises an increasingly greater population of Tc cells. Depending on the purpose of treatment, in some embodiments, infusing a subject with a T cell population comprising predominately of Th cells may be advantageous. In some embodiments, if an antigen-specific subset of Tc cells has been isolated, it may be beneficial to expand this subset to a greater degree.

Further, in addition to CD4 and CD8 markers, other phenotypic markers vary significantly, but in large part, reproducibly during the course of the cell expansion process. Such reproducibility enables the ability to tailor an activated T cell product for specific purposes.

In some embodiments of the invention, the T cells may be cultured in the presence of rapamycin in order to obtain regulatory T cells, as described for example in PCT Patent Publication WO 2007/110785 (incorporated herein by reference). Another method to generate regulatory T cells is described in U.S. Patent Publication 2016/024470 (incorporated herein by reference), wherein T cells are cultured with a T cell receptor (TCR)/CD3 activator such as for example TCR/CD3 antibodies, a TCR co-stimulator activator such as for example CD28, CD137 (4-1 BB), GITR, B7-1/2, CD5, ICOS, OX40, CD40 or CD137 antibodies, and rapamycin.

In some embodiments of the invention, T cells genetically modified by expression of a CAR as described herein may also have been genetically modified by expression of at least one intracellular factor such as ROR-C, FoxP3, Foxo1, T-bet, or Gata 3, c-Maf, or AhR. In some embodiments, the genetically modified immune cell of the invention expresses FoxP3. In some embodiments, the genetically modified immune cell of the invention expresses Foxo1.

In some embodiments, the genetically modified immune cell of the invention may be an allogeneic immune cell. In such instances, the cell may be engineered to reduce host rejection to the cell (graft rejection) and/or the cell's potential attack on the host (graft-versus-host disease). By way of example, the cell may be engineered to have a null genotype for one or more of the following: (i) T cell receptor (TCR alpha chain or beta chain); (ii) a polymorphic major histocompatibility complex (MHC) class I or II molecule (e.g., HLA-A, HLA-B, or HLA-C; HLA-DP, HLA-DM, HLA-DOA, HLA-DOB, HLA-DQ, or HLA-DR; or β2-microglobulin (B2M)); (iii) a transporter associated with antigen processing (e.g., TAP-1 or TAP-2); (iv) Class II MHC transactivator (CIITA); (v) a minor histocompatibility antigen (MiHA; e.g., HA-1/A2, HA-2, HA-3, HA-8, HB-1H, or HB-1Y); and (vi) any combination thereof. The allogeneic engineered cells may also express an invariant HLA or CD47 to protect the engineered Treg cells from host rejection. These further genetic modifications may be performed by the gene editing techniques known in the art and those described herein.

The further-edited allogeneic cells are particularly useful because they can be used in multiple patients without compatibility issues. The allogeneic cells thus can be called “universal” and can be used “off the shelf” The use of “universal” cells greatly improves the efficiency and reduces the costs of adopted cell therapy.

In certain embodiments, the allogeneic immune cell can be engineered such that it does not express any functional TCR on its surface, engineered such that it does not express one or more subunits that comprise a functional TCR or engineered such that it produces very little functional TCR on its surface. For example, an immune cell as described herein can be engineered such that cell surface expression of TCR molecules is downregulated. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

In certain embodiments, the allogeneic immune cell can be engineered such that it does not express a functional HLA on their surface. For example, an immune cell as described herein can be engineered such that cell surface expression of HLA, e.g., HLA class 1 and/or HLA class II and/or non-classical HLA molecules is downregulated.

In certain embodiments, the T cell can lack a functional TCR and a functional HLA such as HLA class I and/or HLA class II.

Modified immune cells that lack expression of a functional TCR and/or HLA can be obtained by any suitable means, including a knock out or knock down of one or more subunit of TCR and/or HLA. For example, the Treg cell can include a knock down of TCR and/or HLA using siRNA, shRNA, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain).

In some embodiments, the nucleic acid encoding a CAR as described herein is inserted at a specific locus in the genome of an immune cell, such as, for example, at the locus of a gene to be deleted. In some embodiments, the nucleic acid encoding a CAR as described herein is inserted within a TCR and/or HLA locus, thereby resulting in the inhibition of TCR and/or HLA expression.

In some embodiments, TCR and/or HLA expression can be inhibited using siRNAs or shRNAs that targets a nucleic acid encoding a TCR and/or HLA in a T cell. Expression of siRNAs and shRNAs in T cells can be achieved using any conventional expression system, e.g., such as a lentiviral expression system. Exemplary siRNAs and shRNAs that downregulate expression of HLA class I and/or HLA class II genes are described, e.g., in U.S. Patent Publication 2007/0036773. Exemplary shRNAs that downregulate expression of components of the TCR are described, e.g., in U.S. Patent Publication 2012/0321667.

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas,” as used herein, refers to a CRISPR-associated protein. A “CRISPR/Cas” system refers to a system derived from CRISPR and Cas that can be used to silence or mutate a TCR and/or HLA gene.

Naturally-occurring CRISPR/Cas systems are found in approximately 40% of sequenced eubacteria genomes and 90% of sequenced archaea. See, e.g., Grissa et al. (BMC Bioinformatics 8:172 (2007)). This system is a type of prokaryotic immune system that confers resistance to foreign genetic elements such as plasmids and phages and provides a form of acquired immunity. See, e.g., Barrangou et al., Science 315:1709-1712 (2007); Marragini et al., Science 322:1843-1845 (2008). The CRISPR/Cas system has been modified for use in gene editing (silencing, enhancing or changing specific genes) in eukaryotes such as mice or primates. See, e.g., Wiedenheft et al., Nature 482: 331-8 (2012). This is accomplished by introducing into the eukaryotic cell a plasmid containing a specifically designed CRISPR and one or more appropriate Cas-encoding sequences. The CRISPR sequence, sometimes called a CRISPR locus, comprises alternating repeats and spacers. In naturally-occurring CRISPR, the spacers usually comprise sequences foreign to the bacterium such as a plasmid or phage sequence; in the TCR and/or HLA CRISPR/Cas system, the spacers are derived from the TCR and/or HLA gene sequence. RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. See, e.g., Horvath et al., Science 327:167-170 (2010); Makarova et al., Biology Direct 1:7 (2006). The spacers thus serve as templates for RNA molecules, analogously to siRNAs. See, e.g., Pennisi, Science 341:833-836 (2013). The CRISPR/Cas system thus can be used to edit a TCR and/or HLA gene (adding or deleting a base pair), or introduce a premature stop that decreases expression of a TCR and/or HLA. The CRISPR/Cas system alternatively or additionally can be used like RNA interference, turning off the TCR and/or HLA gene in a reversible fashion. In a mammalian cell, for example, the RNA can guide the Cas protein to a TCR and/or HLA promoter, sterically blocking RNA polymerases.

Artificial CRISPR/Cas systems can be generated that inhibit TCR and/or HLA, using technology known in the art, e.g., that described in U.S. Patent Publication 2014/0068797, and Cong, Science 339:819-823 (2013). Other artificial CRISPR/Cas systems that are known in the art may also be generated to inhibit TCR and/or HLA, e.g., those described in Tsai, Nature Biotechnol. 32:6569-576 (2014) and U.S. Pat. Nos. 8,871,445; 8,865,406; 8,795,965; 8,771,945; and 8,697,359.

It is understood that the above procedures may also be carried out by CRISPR systems that utilize endonucleases other than Cas, such as Cpf1 and C2c1/2/3.

“TALEN” or “TALEN to TCR and/or HLA” or “TALEN to inhibit TCR and/or HLA” refers to a transcription activator-like effector nuclease, an artificial nuclease that can be used to edit the TCR and/or HLA gene. TALENs are produced artificially by fusing a TAL effector DNA binding domain to a DNA cleavage domain. Transcription activator-like effectors (TALEs) can be engineered to bind any desired DNA sequence, including a portion of the TCR and/or HLA gene. By combining an engineered TALE with a DNA cleavage domain, a restriction enzyme can be produced that is specific to any desired DNA sequence, including a TCR and/or HLA sequence. These can then be introduced into a cell, wherein they can be used for genome editing. See, e.g., Boch, Nature Biotech. 29:135-6 (2011); Boch et al., Science 326:1509-12 (2009); and Moscou et al., Science 326:3501 (2009).

TALEs are proteins secreted by Xanthomonas bacteria. The DNA binding domain contains a repeated 33-34 amino acid sequence that is highly conserved with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. See, e.g., Cermak et al., Nucl. Acids Res. 39:e82 (2011); Miller et al., Nature Biotech. 29:143-8 (2011); Hockemeyer et al., Nature Biotech. 29:731-734 (2011); Wood et al., Science 333:307 (2011); Doyon et al., Nature Methods 8:74-79 (2010); Szczepek et al., Nature Biotech. 25:786-793 (2007); and Guo et al., J. Mol. Biol. 200:96 (2010). The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity (Miller et al., Nature Biotech. 29:143-8 (2011)). A TCR and/or HLA TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the TCR and/or HLA gene or introduce such a defect into a wt HLA gene, thus decreasing expression of TCR and/or HLA. TALENs specific to sequences in TCR and/or HLA can be constructed using any method known in the art, including various schemes using modular components (Zhang et al., Nature Biotech. 29:149-53 (2011); Geibler et al., PLoS ONE 6:e19509 (2011)).

“ZFN” or “Zinc Finger Nuclease” or “ZFN to TCR and/or HLA” or “ZFN to inhibit TCR and/or HLA” refers to a zinc finger nuclease, an artificial nuclease that can be used to edit the TCR and/or HLA gene. Like a TALEN, a ZFN comprises a Fokl nuclease domain (or derivative thereof) fused to a DNA-binding domain. In the case of a ZFN, the DNA-binding domain comprises one or more zinc fingers. See, e.g., Carroll et al., Genetics Society of America 188:773-782 (2011); and Kim et al., Proc. Natl. Acad. Sci. USA 93:1156-1160 (1996). A zinc finger is a small protein structural motif stabilized by one or more zinc ions. A zinc finger can comprise, for example, Cys₂His₂, and can recognize an approximately 3 bp sequence. Various zinc fingers of known specificity can be combined to produce multi-finger polypeptides that recognize about 6, 9, 12, 15 or 18 bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs is required to target non-palindromic DNA sites. The two individual ZFNs must bind opposite strands of the DNA with their nucleases properly spaced apart (Bitinaite et al., Proc. Natl. Acad. Sci. USA 95:10570-5 (1998)). Also like a TALEN, a ZFN can create a double-stranded break in the DNA, which can create a frame-shift mutation if improperly repaired, leading to a decrease in the expression and amount of TCR and/or HLA in a cell. ZFNs can also be used with homologous recombination to mutate the TCR and/or HLA gene. ZFNs specific to sequences in TCR and/or HLA can be constructed using any method known in the art. See, e.g., Provasi, Nature Med. 18:807-815 (2011); Torikai, Blood 122:1341-1349 (2013); Cathomen et al., Mol. Ther. 16:1200-7 (2008); Quo et al., J. Mol. Biol. 400:96 (2010); and U.S. Patent Publications 2011/0158957 and 2012/0060230.

“Meganuclease” or “meganuclease to TCR and/or HLA” or “meganuclease to inhibit TCR and/or HLA” refers to a monomeric endonuclease with large (>14 base pairs) recognition sites, which can be used to edit the TCR and/or HLA gene. Meganucleases (mn) are monomeric proteins with innate nuclease activity that are derived from bacterial homing endonucleases and engineered for a unique target site. Homing endonucleases are DNA-cleaving enzymes that can generate double strand breaks at individual loci in their host genomes, and thereby drive site-specific gene conversion events (Stoddard, Structure 19(1):7-15 (2011)). Despite their small size, homing endonucleases recognize long DNA sequences (typically 20 to 30 base pairs). Homing endonucleases are extremely widespread and are found in microbes, as well as in phages and viruses. The LAGLIDADG and His-Cys box enzymes (which are the most sequence-specific of these enzymes) rely upon antiparallel (3-sheets that dock into the major grooves of their DNA target sites (Flick et al., Nature 394(6688):96-101 (1998); Jurica et al., Mol. Cell. 2(4):469-76 (1998). There they establish a collection of sequence-specific and non-specific contacts that are distributed nonuniformly across multiple consecutive basepairs (Chevalier et al., J Mol Biol 329(2):253-269 (2003); Scalley-Kim et al., J Mol Biol. 372(5):1305-19 (2007).

The LAGLIDADG homing endonuclease (LHE) family is the primary source of the engineered enzymes used for gene targeting applications. The LHE family is primarily encoded within archaea and in the chloroplast and mitochondrial genomes of algae and fungi (Chevalier et al., in Homing Endonucleases and Inteins. Nucleic Acids and Molecular Biology, vol. 16 (2005); Dalgaard et al., Nucleic Acids Res. 25(22):4626-38 (1997); Sethuraman et al., Mol Biol Evol. 26(10):2299-315 (2009). Meganucleases that possess a single conserved LAGLIDADG motif (SEQ ID NO: 58) per protein chain form homodimeric proteins that cleave palindromic and nearly palindromic DNA target sequences, while those that contain two such motifs per protein chain form larger, pseudo-symmetric monomers that can target completely asymmetric DNA sequences.

Meganucleases can be engineered to target TCR and/or HLA and thus create a double-stranded break in the DNA, which can create a frame-shift mutation if improperly repaired, leading to a decrease in the expression and amount of TCR and/or HLA in a cell.

“MegaTAL” or “megaTAL to TCR and/or HLA” or “megaTAL to inhibit TCR and/or HLA” refers to an artificial nuclease, which can be used to edit the TCR and/or HLA gene. MegaTALs are hybrid monomeric nucleases obtained through the fusion of minimal TAL (transcription activator-like) effector domains to the N-terminus of meganucleases derived from the LAGLIDADG homing endonuclease family (Boissel et al., Nucleic Acids Res. 42(4):2591-601 (2014); Takeuchi et al, Methods Mol Biol. 1239:105-32 (2015)). MegaTALs thus consist of a site-specific meganuclease cleavage head with additional affinity and specificity provided by a TAL effector DNA binding domain.

MegaTALs can be engineered to target TCR and/or HLA and thus create a double-stranded break in the DNA, which can create a frame-shift mutation if improperly repaired, leading to a decrease in the expression and amount of TCR and/or HLA in a cell. A variant of the I-OnuI meganuclease (mn) was used to design a TCRα-megaTAL to knockout the T-cell receptor alpha (TCRα) gene. The TCRa mn was fused to a 10.5 repeat TALE array designed to bind a DNA sequence upstream of the TCRa mn binding site. It was found that the megaTAL targeting TCRa achieved extremely high gene disruption with no detectable off-target cleavage in human primary T-cells (Boissel et al, Nucleic Acids Res 42(4):2591-601 (2014)).

In some embodiments, a T cell population expressing a CAR of the invention is one that lacks an endogenous T cell receptor (TCR) or has been altered to reduce or eliminate the expression or activity of the endogenous TCR. It is understood that any of the methods described herein for inhibiting or eliminating HLA expression may also be used to target one or more components of the endogenous TCR.

In some embodiments, transfection with a telomerase gene can lengthen the telomeres of a T cell and improve persistence of the T cell in the patient. See, e.g., June, Journal of Clinical Investigation 117: 1466-1476 (2007). Thus, in some embodiments, a genetically modified Treg cell of the invention ectopically expresses a telomerase subunit, e.g., the catalytic subunit of telomerase, e.g., TERT, e.g., hTERT. In some aspects, this disclosure provides a method of producing a chimeric receptor-expressing cell of the invention, comprising contacting the cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding the chimeric receptor (e.g., a CAR as described herein).

The present invention further relates to a method for obtaining an immune cell of the invention, wherein said method comprises transducing at least one immune cell with a nucleic acid encoding a CAR as described herein, and optionally expanding the transduced cells. In some embodiments, the method is an ex vivo method.

In some embodiments, the method for obtaining immune cells of the invention comprises:
a step of isolating/enriching immune cells from a PBMC population (e.g., recovered by leukapheresis),
optionally an activation step,
a transduction step with a vector comprising a nucleic acid sequence encoding a CAR as described herein,
optionally an expansion step,
optionally a washing step and,
optionally a freezing step.

In some embodiments, engineered Treg cells of the invention may be cultured in tissue culture media containing rapamycin and/or a high concentration of IL-2 to maintain the Treg phenotype and/or to increase expression of FoxP3 and the transgene.

### Compositions

Another object of the invention is a composition comprising, consisting or consisting essentially of least one immune cell or immune cell population of the invention as described herein. In some embodiments, said immune cell or immune cell population of the invention is selected from the group consisting of T cells, natural killer (NK) cells, γδ T cells, double negative (DN) cells, regulatory immune cells, regulatory T cells, effector immune cells, effector T cells, B cells and myeloid-derived cells, and any combination thereof.

As used herein, the term “consisting essentially of,” in reference to a pharmaceutical composition or medicament, means that the at least one immune cell or cell population of the invention is the only therapeutic agent or agent with a biologic activity within said pharmaceutical composition or medicament.

In some embodiments, said composition comprises, consists or consists essentially of at least one isolated and/or substantially purified immune cell population of the invention as described herein.

In some embodiments, said composition comprises, consists or consists essentially of at least one immune cell population of the invention, as described herein, engineered to express at the cell surface a CAR described herein (e.g., a CAR comprising: at least one extracellular binding domain, optionally at least one extracellular hinge domain, at least one transmembrane domain, and at least one intracellular domain, wherein the at least one intracellular domain comprises at least one primary intracellular signaling domain and optionally at least one costimulatory intracellular signaling domain), wherein the at least one transmembrane domain is a human TNFR2 transmembrane domain or a fragment or variant thereof or any transmembrane domain or a fragment or variant thereof or a combination thereof, and/or the at least one costimulatory intracellular signaling domain is a human TNFR2 costimulatory intracellular signaling domain or a fragment or variant thereof or any costimulatory intracellular signaling domain or a fragment or variant thereof or a combination thereof, wherein at least one of the transmembrane domain and costimulatory intracellular signaling domain is a TNFR2 transmembrane domain or a fragment or variant thereof or a TNFR2 costimulatory intracellular signaling domain or a fragment or variant thereof.

In some embodiments, said composition has been frozen and thawed.

Another object of the invention is a pharmaceutical composition comprising, consisting or consisting essentially of at least one immune cell or population as described herein and at least one pharmaceutically acceptable excipient.

Another object of the invention is a medicament comprising, consisting or consisting essentially of at least one immune cell population as described herein.

In some embodiments, the pharmaceutical composition or medicament comprises at least one isolated and/or substantially purified immune cell population of the invention as described herein.

In some embodiments, the pharmaceutical composition or medicament comprises a combination of immune cell populations as described herein (i.e., at least two distinct immune cell populations of the invention).

In some embodiments, the composition, pharmaceutical composition or medicament of the invention further comprises at least a second, distinct immune cell population, wherein cells of said second immune cell population express on the cell surface a second, distinct CAR specific for an antigen, a fragment of an antigen, a variant of an antigen or a mixture thereof. In some embodiments, the second CAR is specific for a food antigen from the common human diet. In some embodiments, the second CAR is specific for an autoantigen, such as, for example, a multiple sclerosis-associated antigen, a joint-associated antigen, an eye-associated antigen, a human HSP antigen, a skin-associated antigen or an antigen involved in graft rejection or GVHD. In some embodiments, the second CAR is specific for an inhaled allergen, an ingested allergen or a contact allergen. In some embodiments, the second CAR is specific for an antigen selected from the group consisting of ovalbumin, MOG, type II collagen, citrullinated vimentin, citrullinated type II collagen, citrullinated fibrinogen, and fragments, variants and mixtures thereof.

In some embodiments, the second CAR is specific for ovalbumin or a fragment, variant, or mixture thereof. In some embodiments, the second CAR is specific for MOG or a fragment, variant, or mixture thereof. In some embodiments, the second CAR is specific for type II collagen or a fragment, variant, or mixture thereof. In some embodiments, the second CAR is specific for citrullinated vimentin, citrullinated type II collagen or citrullinated fibrinogen, or fragments, variants and mixtures thereof. In some embodiments, the second CAR is specific for HLA-A2 or a fragment, variant, or mixture thereof. In some embodiments, the second CAR is specific for IL-23R or a fragment, variant, or mixture thereof. In some embodiments, the second CAR is specific for a B cell surface marker, such as, for example, CD19, CD20, or fragments, variants and mixtures thereof. In some embodiments, the second CAR is specific for a cancer antigen as described herein or a fragment, variant, or mixture thereof.

In some embodiments, the second CAR recognizes infected cells. In some embodiments, the second CAR is specific for a viral antigen or a fragment, variant, or mixture thereof; a bacterial antigen or a fragment, variant, or mixture thereof; or a fungal antigen or a fragment, variant, or mixture thereof.

In some embodiments, the cells of said second immune cell population express on the cell surface a CAR wherein the extracellular binding domain of said CAR is a protein or a fragment or variant thereof, such as for example, an autoantigen or a fragment or variant thereof.

In some embodiments, the cells of said other immune cell population express on the cell surface a CAR specific for an autoantibody, such as for example, an autoantibody expressed on a B cell.

The compositions and medicaments described herein may comprise e.g., buffers such as sterilized water, physiological saline, neutral buffered saline, phosphate buffered saline and the like; salts; antibiotics; isotonic agents; carbohydrates such as glucose, mannose, sucrose, dextrans, and mannitol; proteins such as human serum albumin); polypeptides; amino acids such as glycine and arginine; antioxidants; chelating agents such as EDTA or glutathione; adjuvants (e.g., aluminum hydroxide); and preservatives. The compositions and medicaments may additionally comprise factors that are supportive of the Treg phenotype and growth (e.g., IL-2 and rapamycin or derivatives thereof), anti-inflammatory cytokines (e.g., IL-10, TGF-β, and IL-35), and other cells for cell therapy (e.g., CAR T effector cells for cancer therapy or cells for regenerative therapy). For storage and transportation, the cells optionally may be cryopreserved. Prior to use, the cells may be thawed and diluted in a pharmaceutically acceptable carrier.

The compositions of the present invention are in some embodiments formulated for intravenous administration.

In some embodiments, cells expressing CARs of the invention exhibit reduced tonic signaling as compared to cells expressing CARs without a TNFR2 transmembrane domain or a fragment or variant thereof and/or without a TNFR2 intracellular costimulatory signaling domain or a fragment or variant thereof.

The term “tonic signaling” as used herein refers to an antigen-independent background of activation.

Methods for measuring tonic signaling are well known to the skilled artisan, and include, without limitation, measuring metabolic activity of the CAR-expressing cells, measuring one or more indicators of cell activation in the absence of stimulation by an antigen recognized by the receptor, measuring one or more phenotypical changes related to cell aging or cell senescence, determining cell cycle progression in the absence of antigenic stimulation; and measuring the size of cells expressing the receptor compared to the size of unmodified cells.

In some embodiments, the CAR-engineered immune cells expressing a TNFR2 transmembrane domain or a fragment or variant thereof and/or a TNFR2 intracellular costimulatory signaling domain or a fragment or variant thereof have reduced tonic signaling as compared to CAR-engineered immune cells that do not express a TNFR2 transmembrane domain or a fragment or variant thereof and/or a TNFR2 intracellular costimulatory domains or a fragment or variant thereof when measured in the conditions of TEST A.

### Test A:

An indicator of T cell activation is used to gauge of tonic signaling. In the conditions of TEST A, the level of CD69 positive cells is measured in the cell population before and after activation of the CAR of the invention. The activation assay is performed at day 9 of the immune cell culture. Briefly, 0.05×10⁶ immune cells are seeded in 96 U bottom plates alone or in the presence of anti-CD28/anti-CD3 coated beads (in a 1:1 immune cell to beads ratio), or in the presence of freshly thawed autologous B cells (in a 1:1 immune cell to B cell ratio) in a 200 μl final volume. After 24 h at 37° C., 5% CO2, cells are stained for CD69 and a marker related to a specific immune cell population (e.g., CD19 for B cells, or CD4 or CD8 for T cells) and then analyzed using flow cytometry. The monitoring of CD69 spontaneous expression (meaning without any antigenic simulation) by cells expressing a CAR described herein (e.g., comprising a TNFR2 transmembrane domain or a fragment or variant thereof and/or a TNFR2 intracellular costimulatory signaling domain or a fragment or variant thereof), as compared to untransduced cells, allows determination of tonic signaling intensity. Cells transduced with a CAR that does not comprise a TNFR2 transmembrane or TNFR2 intracellular costimulatory signaling domain are used as a control.

In some embodiments, the CAR constructs of the invention may be used to produce an engineered immune cell with reduced tonic signaling. In some embodiments, the present invention provides a method to produce an engineered immune cell (i.e., a cell expressing a CAR) with reduced tonic signaling. Currently available CAR constructs, especially those used in the CAR effector field, are often associated with a high tonic signaling, limiting the potency of CAR engineered cells. Indeed, such tonic signaling is associated with constitutive activation of CAR engineered cells, leading to their premature exhaustion or even death. The CAR constructs of the invention (comprising a TNFR2 transmembrane domain or a fragment or variant thereof and/or a TNFR2 costimulatory intracellular signaling domain or a fragment or variant thereof) provide diminished tonic signaling and constitutive activation of cells.

### Therapeutic Uses

The invention provides a method for treating one or more diseases, disorders, symptoms, or condition in a subject in need thereof, wherein said method comprises administering to the subject a CAR-engineered immune cell or composition as described herein. The present invention also provides at least one immune cell population as described herein (e.g., in a composition, pharmaceutical composition or medicament as described herein) for use as a medicament. The present invention also provides at least one immune cell population as described herein (e.g., in a composition, pharmaceutical composition or medicament as described herein) for use in the preparation of a medicament. Diseases that may be treated with engineered immune cells of the invention include, but are not limited to, inflammatory diseases, autoimmune diseases, allergic diseases, organ transplantation conditions, cancer and infectious diseases.

In some embodiments, the method for treating a disease or disorder in a subject in need thereof comprises administering to the subject at least one immune cell population as described herein, e.g., an engineered regulatory immune cell population (such as a Treg cell population), wherein said disease or disorder is an inflammatory disease, an autoimmune disease, an allergic disease, or an organ transplantation condition. In certain embodiments, the disease or disorder is graft rejection or graft-versus-host disease.

In some embodiments, the method for treating a disease or disorder in a subject in need thereof comprises administering to the subject at least one immune cell population as described herein, e.g., an engineered effector immune cell population (such as a Treg cell population), wherein said disease or disorder is a cancer or an infectious disease.

In some embodiments, the method for treating a disease or disorder in a subject in need thereof comprises administering to the subject at least one immune cell population as described herein, e.g., an engineered effector immune cell population (such as a Treg cell population), wherein said subject is in need of gene therapy (e.g., AAV-based gene therapy) for said disease or disorder.

In some embodiments, the method is a cell therapy method.

In some embodiments, the cell therapy is autologous.

In some embodiments, the cell therapy is heterologous.

In some embodiments, the cell therapy is allogenic.

In some embodiments, the method is a gene therapy method and involves administration of a nucleic acid or vector as described herein.

### 1. Inflammatory Diseases

In some embodiments, the CAR-engineered immune cells of the invention may be used in the treatment of one or more inflammatory diseases, disorders, symptoms, or conditions in a subject in need thereof. In certain embodiments, the CAR-engineered immune cells of the invention may be used to promote immune tolerance in this context. The present invention provides a method of treating an inflammatory disease or disorder in a subject in need thereof, wherein said method comprises administering a therapeutically effective amount of at least one immune cell population as described herein. The present invention also relates to at least one immune cell population as described herein (e.g., in a composition, pharmaceutical composition or medicament as described herein) for use in the treatment of an inflammatory disease or disorder. The present invention also provides at least one immune cell population as described herein (e.g., in a composition, pharmaceutical composition or medicament as described herein), for use in the preparation of a medicament for treating an inflammatory disease or disorder.

The terms “inflammatory disorder” and “inflammatory disease” are used interchangeably and as used herein refer to any abnormality associated with inflammation.

In some embodiments, the inflammatory condition comprises inflammatory diseases and inflammation linked to an infection or linked to cancer.

In some embodiments, the inflammatory condition comprises inflammatory diseases and inflammation linked to an autoimmune diseases.

Exemplary inflammatory diseases, disorders, or conditions include, but are not limited to, arthritis, rheumatoid arthritis, ankylosing spondylitis, osteoarthritis, psoriatic arthritis, juvenile idiopathic arthritis, juvenile rheumatoid arthritis, arthritis uratica, gout, chronic polyarthritis, periarthritis humeroscapularis, cervical arthritis, lumbosacral arthritis, enteropathic arthritis and ankylosing spondylitis, asthma, dermatitis, psoriasis, scleroderma, polymyositis, dermatomyositis, juvenila dermatomyositis, primary biliary cirrhosis, fibrosis, cystic fibrosis, pulmonary fibrosis, cirrhosis, endomyocardial fibrosis, dediastinal fibrosis, myelofibrosis, retroperitoneal fibrosis, nephrogenic fibrosis, keloids, scleroderma, arthrofibrosis, post transplantation late and chronic solid organ rejection, multiple sclerosis, systemic lupus erythematosus, lupus nephritis, pemphigus, pemphigus vulgaris, pemphigus herpetiformis, pemphigus vegetans, IgA pemphigus, pemphigus erythematosus, bullous pemphigoid, pemphigoid gestationis, mucous membrane dermatosis, pemphigoid nodularis, linear IgA bullous dermatosis, bullous lichen planus, epidermolysis bullosa acquisita, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, small artery disease, acute disseminated encephalomyelitis, idiopathic thrombocytopenic purpura, multiple sclerosis, systemic sclerosis, antiphospholipid syndrome, Sjoegren's syndrome, autoimmune hemolytic anemia, colitis, Crohn's disease, ulcerative colitis, inflammatory bowel disease (IBD), embolism, pulmonary embolism, arterial embolism, venous embolism, allergic inflammation, cardiovascular disease, graft-related diseases, graft versus host disease (GVHD), disorders associated with graft transplantation rejection, chronic rejection, and tissue or cell allografts or xenografts, autoimmune diseases, degeneration after trauma, stroke, transplant rejection, allergic conditions and hypersensitivity, e.g., allergic rhinitis, allergic eczema and the like, skin diseases, dermal inflammatory disorders, and any combination thereof.

Examples of skin diseases include, but are not limited to, acne; actinic keratosis; atopic dermatitis; contact dermatitis; decubitus ulcers (bedsores); eczema; erythroderma; hemangioma, such as, for example, hemangioma of childhood; hypertrophic scarring; lichen planus; lichenoid disorders; lymphangiogenesis; psoriasis; pyogenic granulomas; molluscum contagious; neurofibromatosis; rosacea; recessive dystrophic epidermolysis bullosa; scars (keloids); scleroderma; seborrheic keratosis; skin cancers such as angiosarcoma, basal cell carcinoma, hemangioendothelioma, Karposi's sarcoma, malignant melanoma, melanoma, squamous cell carcinoma; skin ulcers; skin damages following skin grafts such as autotransplantation and allotransplantation; Steven-Johnson syndromes and toxic epidermal necrolysis; Sturge-Weber syndrome; tuberous sclerosis; venous ulcers; verruca vulgaris; warts, such as, for example, viral warts; wounds; and the like.

Examples of dermal inflammatory disorders include, but are not limited to, psoriasis, guttate psoriasis, inverse psoriasis, pustular psoriasis, erythroderma psoriasis, acute febrile neutrophilic dermatosis, eczema, asteatotic eczema, dyshidrotic eczema, vesicular palmoplanar eczema, acne vulgaris, atopic dermatitis, contact dermatitis, allergic contact dermatitis, dermatomyositis, exfoliative dermatitis, hand eczema, pompholyx, rosacea, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, Sweet's disease, neutrophilic hidradenitis, sterile pustulosis, drug eruptions, seborrheic dermatitis, pityriasis rosea, cutaneous kikuchi disease, pruritic urticarial papules and plaques of pregnancy, Stevens-Johnson syndrome and toxic epidermal necrolysis, tattoo reactions, Wells syndrome (eosinophilic cellulitis), reactive arthritis (Reiter's syndrome), bowel-associated dermatosis-arthritis syndrome, rheumatoid neutrophilic dermatosis, neutrophilic eccrine hidradenitis, neutrophilic dermatosis of the dorsal hands, balanitis circumscripta plasmacellularis, balanoposthitis, Behcet's disease, erythema annulare centrifugum, erythema dyschromicum perstans, erythema multiforme, granuloma annulare, hand dermatitis, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

### 2. Autoimmune Diseases

In some embodiments, the CAR-engineered immune cells of the invention may be used in the treatment of one or more autoimmune diseases, disorders, symptoms, or conditions in a subject in need thereof. In certain embodiments, CAR-modified immune cells of the invention may be used to promote immune tolerance in this context. The present invention thus provides a method of treating an autoimmune disease in a subject in need thereof, wherein said method comprises administering a therapeutically effective amount of at least one immune cell population as described herein. The present invention also provides at least one immune cell population as described herein (e.g., in a composition, pharmaceutical composition or medicament as described herein) for use in the treatment of an autoimmune disease. The present invention also provides at least one immune cell population as described herein (e.g., in a composition, pharmaceutical composition or medicament as described herein) for use in the manufacture of a medicament for treating an autoimmune disease.

Examples of autoimmune diseases include, but are not limited to, lupus (e.g., lupus erythematosus, lupus nephritis, etc.), Hashimoto's thyroiditis, primary myxedema, Graves' disease, pernicious anemia, autoimmune atrophic gastritis, Addison's disease, diabetes (e.g. insulin dependent diabetes mellitus, type I diabetes mellitus, type II diabetes mellitus, etc.), Goodpasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulcerative colitis, Sjogren's syndrome, rheumatic diseases (e.g., rheumatoid arthritis), polymyositis, scleroderma, psoriasis, mixed connective tissue disease, and the like.

### 3. Allergic Diseases

In some embodiments, the CAR-engineered immune cells of the invention may be used in the treatment of one or more allergic disease, disorders, symptoms, or conditions in a subject in need thereof. In certain embodiments, CAR-modified immune cells of the invention may be used to promote immune tolerance in this context. The present invention thus provides a method of treating an allergic disease, disorder, symptom, or condition in a subject in need thereof, wherein said method comprises administering a therapeutically effective amount of at least one immune cell population as described herein. The present invention also provides at least one immune cell population as described herein (e.g., in a composition, pharmaceutical composition or medicament as described herein) for use in the treatment of an allergic disease, disorders, symptoms, or conditions. The present invention also provides at least one immune cell population as described herein (e.g., in a composition, pharmaceutical composition or medicament as described herein) for use in the manufacture of a medicament for treating an allergic disease, disorders, symptoms, or conditions.

Examples of allergic diseases include, but are not limited to, allergic diseases against an inhaled allergen, an ingested allergen or a contact allergen. Other examples of allergic diseases include, but are not limited to, allergic asthma, hypersensitivity lung diseases, food allergy, atopic dermatitis, allergic rhinitis, allergic rhinoconjunctivitis, chronic urticaria, delayed-type hypersensitivity disorders and systematic anaphylaxis.

### 4. Transplantation

In some embodiments, the CAR-engineered immune cells of the invention may be used in the treatment of one or more diseases, disorders, symptoms, or conditions associated with organ or tissue transplant (e.g., organ or tissue rejection/dysfunction, GVHD, and/or conditions associated therewith). Transplant rejection involves the destruction of the donor's transplanted tissue by the recipient's immune cells through an immune response. An immune response is also involved in GVHD; however, in this case, the recipient's tissues are destroyed by the donor's immune cells transferred to the recipient via the transplant. Accordingly, CAR-mediated redirection and activation of immune cells provide a method of suppressing rejection of mismatched cells and/or tissues by immune effector cells in transplant recipients or inhibiting the pathogenic action of transplanted immunocompetent cells in the case of GVHD. In some embodiments, the mismatched cells and/or tissues comprise HLA-A2 mismatched cells and/or tissues. The CAR-modified immune cells of the invention may be used to promote immune tolerance, operational tolerance, and/or immune accommodation in a subject, in particular following organ or tissue transplantation. The present invention thus provides a method of promoting immune tolerance, operational tolerance, and/or immune accommodation in a subject, the method comprising administering to the subject a CAR-engineered immune cell, or a pharmaceutical composition, as described herein. In some embodiments, the method may be for promoting immune tolerance, operational tolerance, and/or immune accommodation to a transplanted organ or tissue in a subject. The present invention also provides at least one immune cell population as described herein (e.g., in a composition, pharmaceutical composition or medicament as described herein) for use in promoting immune tolerance, operational tolerance, and/or immune accommodation to a transplanted organ or tissue in a subject or to a transplanted organ or tissue in a subject. The present invention also provides at least one immune cell population as described herein (e.g., in a composition, pharmaceutical composition or medicament as described herein) for use in the manufacture of a medicament for promoting immune tolerance, operational tolerance, and/or immune accommodation to a transplanted organ or tissue in a subject or to a transplanted organ or tissue in a subject.

In some embodiments, a CAR-engineered immune cell (e.g., a CAR-engineered Treg cell) of the invention is administered at the same time as, before, or after the transplantation of the organ or tissue.

In some embodiments, a CAR-engineered immune cell (e.g., a CAR-engineered Treg cells) of the invention may be used to prevent or treat rejection of a transplanted organ or tissue. Examples of rejection of a transplanted organ or tissue include, but are not limited to, hyperacute rejection of a transplanted organ or tissue, and antibody-mediated rejection of a transplanted organ or tissue.

In some embodiments, the method of the invention comprises administering CAR-engineered immune cells (e.g., CAR-engineered Treg cells) of the present invention to a subject exposed to a transplanted organ or tissue.

In some embodiments, the transplanted organ or tissue may encompass a bone marrow transplant, an organ transplant, a blood transfusion or any other foreign tissue or cell that is purposefully introduced into a subject.

In some embodiments, CAR-engineered immune cells (e.g., CAR-engineered Treg cells) of the present invention may be used as a therapy to inhibit graft rejection following transplantation, including, without limitation, allograft rejection or xenograft rejection.

Another object of the invention is a method of preventing or treating organ or tissue transplant rejection in a subject, the method comprising administering to the subject CAR-engineered immune cells (e.g., CAR-engineered Treg cells) of the invention, or a pharmaceutical composition comprising said immune cells.

Another object of the invention is a method of increasing the time period of graft survival in a subject, the method comprising administering to the subject CAR-engineered immune cells (e.g., CAR-engineered Treg cells) of the present invention, or a pharmaceutical composition comprising the same.

In some embodiments, the method provides a time period of graft survival of 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 20 years, 30 years, 40 years, 50 years, 60 years, 70 years, 80 years, 90 years, 100 years, or the lifetime of the subject.

In some embodiments, the administration of an immune cell or composition of the invention allows reduction of the amount of an immunosuppressant agent therapy received by the subject. In some embodiments, the subject does not require, and/or is not undergoing, any immunosuppressant agent therapies.

In some embodiments, the graft is an allograft. In some embodiments, the transplant may be exposed to CAR-engineered immune cells (e.g., CAR-engineered Treg cells) of the present invention at the same time as, before, or after transplantation of the transplant into the recipient. In some embodiments, the organ or tissue transplant may be a heart, heart valve, lung, kidney, liver, pancreas, intestine, skin, blood vessels, bone marrow, stem cells, bone, or, islet cells. However, the invention is not limited to a specific type of transplantation.

In some embodiments, the donor transplant may be “preconditioned” or “pretreated” by treating the organ or tissue transplant prior to transplantation into the recipient with CAR-engineered immune cells of the invention in order to reduce the immunogenicity of the transplant against the recipient, thereby reducing or preventing graft rejection.

In some embodiments, the transplant host or recipient is HLA-A2 negative. In some embodiments, the transplant host or recipient is HLA-A2 negative and is positive for an HLA-A subtype selected from the group consisting of HLA-A25, HLA-A29 and HLA-A30.

In some embodiments, the transplant is HLA-A2 positive.

In some embodiments, the CAR-engineered immune cells (e.g., CAR-engineered Treg cells) of the present invention may be used to prevent or treat graft versus host disease (GVHD). In certain embodiments, the GVHD may occur after hematopoietic stem cell transplantation. In some embodiments, the method comprises administering CAR-engineered immune cells (e.g., CAR-engineered Treg cells) of the present invention to a subject exposed to a transplanted organ or tissue. In some embodiments, the transplanted organ or tissue may encompass a bone marrow transplant, an organ transplant, a blood transfusion, or any other foreign tissue or cell that is purposefully introduced into a subject. For example, GVHD may occur after heart, heart valve, lung, kidney, liver, pancreas, intestine, skin, blood vessel, bone marrow, stem cell, bone or islet cell transplantation. However, the invention is not limited to a specific type of transplantation.

Another object of the invention is a method of preventing or treating graft versus host disease (GVHD) in a subject, the method comprising administering to the subject CAR-engineered immune cells (e.g., CAR-engineered Treg cells) or a pharmaceutical composition as described herein.

In some embodiments, the invention provides a method of contacting a donor transplant, for example, a biocompatible lattice or a donor tissue, organ or cell, with CAR-engineered immune cells (e.g., CAR-engineered Treg cells) of the present invention at the same time as, before, or after the transplantation of the transplant into a recipient.

In some embodiments, the CAR-engineered immune cells (e.g., CAR-engineered Treg cells) of the present invention may be used to ameliorate, inhibit or reduce an adverse response by the donor transplant against the recipient, thereby preventing or treating GVHD.

Another object of the present invention is a method of preventing or delaying onset of GVHD in a subject, the method comprising administering to the subject CAR-engineered immune cells (e.g., CAR-engineered Treg cells) or a pharmaceutical composition as described herein.

In some embodiments, the onset of GVHD is delayed for 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 20 years, 30 years, 40 years, 50 years, 60 years, 70 years, 80 years, 90 years, 100 years, or the lifetime of the subject.

In some embodiments, the administration of an immune cell or composition of the invention allows reduction of the amount of an immunosuppressant agent therapy received by the subject. In some embodiments, the subject does not require, and/or is not undergoing, any immunosuppressant agent therapies.

In some embodiments, the GVHD may be acute GVHD or chronic GVHD.

In some embodiments, the donor transplant may be “preconditioned” or “pretreated” by treating the transplant prior to transplantation into the recipient with CAR-engineered immune cells (e.g., CAR-engineered Treg cells) of the invention in order to reduce the immunogenicity of the transplant against the recipient, thereby reducing or preventing GVHD. In some embodiments, the transplant may be contacted with cells or a tissue from the recipient prior to transplantation in order to activate T cells that may be associated with the transplant. Following the treatment of the transplant with cells or a tissue from the recipient, the cells or tissue may be removed from the transplant. The treated transplant may then be contacted with CAR-engineered immune cells (e.g., CAR-engineered Treg cells) of the present invention to reduce, inhibit or eliminate the activity of the immune effector cells that were activated by the treatment with the cells or tissue from the recipient. Following this treatment, the CAR-engineered immune cells may be removed from the transplant prior to transplantation into the recipient. However, some CAR-engineered immune cells may adhere to the transplant, and therefore, may be introduced to the recipient with the transplant. In this situation, the CAR-engineered immune cells introduced into the recipient may suppress an immune response against the recipient caused by a cell associated with the transplant.

In some embodiments, the transplant host or recipient is HLA-A2 negative. In some embodiments, the transplant host or recipient is HLA-A2 negative and is positive for an HLA-A subtype selected from the group consisting of HLA-A25, HLA-A29 and HLA-A30.

In some embodiments, the transplant is HLA-A2 positive.

The immune cells may be obtained from any source. For example, in some embodiments, immune cells may be obtained from the tissue donor, the transplant recipient or an otherwise unrelated source (e.g., a different individual or species altogether) for generation of CAR-modified immune cells of the present invention. Accordingly, CAR-modified immune cells of the present invention may be autologous, allogeneic or xenogeneic to the transplant recipient or from an otherwise unrelated source. In some embodiments, the CAR-modified immune cells are CAR-Treg cells that may be autologous, allogeneic or xenogeneic to the transplant recipient. In some embodiments, the CAR-Treg cells may be autologous to the transplant recipient.

### 5. Cancers

In some embodiments, the CAR-engineered immune cells of the invention may be used in the treatment of one or more cancers in a subject in need thereof. In certain embodiments, CAR-engineered immune cells of the invention may be used to promote a specific immune response against cancer cells. The present invention thus provides a method of treating a cancer in a subject in need thereof, wherein said method comprises administering a therapeutically effective amount of at least one immune cell population as described herein. The present invention also provides at least one immune cell population as described herein (e.g., in a composition, pharmaceutical composition or medicament as described herein) for use in the treatment of cancer. The present invention also provides at least one immune cell population as described herein (e.g., in a composition, pharmaceutical composition or medicament as described herein) for use in the manufacture of a medicament for treating cancer.

As used herein, a “cancer” may be any cancer that is associated with a surface antigen or cancer marker.

Examples of cancers include, but are not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, Burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous T cell lymphoma, embryonal tumors, endometrial cancer, ependymoblastoma, ependymoma, esophageal cancer, esthesioneuroblastoma, Ewing sarcoma family of tumors, extracranial germ cell tumor, extragonadal germ cell tumor, extrahepatic bile duct cancer, eye cancer fibrous histiocytoma of bone and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone, medulloblastoma, medulloepithelioma, melanoma, Merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), myeloid leukemia, acute myeloid leukemia (AML), multiple myeloma, myeloproliferative disorders, nasal cavity and paranasal sinus cancer, nasopharyngeal cancer, neuroblastoma, non-Hodgkin lymphoma, non-small cell lung cancer, oral cancer, oral cavity cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, Sezary syndrome, small cell lung cancer, small intestine cancer, soft tissue sarcoma, squamous cell carcinoma, squamous neck cancer, stomach (gastric) cancer, supratentorial primitive neuroectodermal tumors, T cell lymphoma, cutaneous cancer, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenstrom macroglobulinemia, and Wilms Tumor.

In some aspects, the cancer is a B cell malignancy. Examples of B cell malignancies include, but are not limited to, non-Hodgkin's lymphomas (NHL), diffuse large B cell lymphoma (DLBCL), small lymphocytic lymphoma (SLL/CLL), mantle cell lymphoma (MCL), follicular lymphoma (FL), marginal zone lymphoma (MZL), extranodal (e.g., MALT) lymphoma, nodal (e.g., monocytoid B cell) lymphoma, splenic lymphoma, diffuse large cell lymphoma, B cell chronic lymphocytic leukemia/lymphoma, Burkitt's lymphoma and lymphoblastic lymphoma.

### 6. Infectious Diseases

In some embodiments, the CAR-engineered immune cells of the invention may be used in the treatment of one or more infectious diseases, disorders, symptoms, or conditions in a subject in need thereof. In certain embodiments, CAR-modified immune cells of the invention may be used to promote immune tolerance in this context. In some embodiments, the present invention provides a method of treating an infectious disease in a subject in need thereof, wherein said method comprises administering a therapeutically effective amount of at least one immune cell population as described herein. The present invention also provides at least one immune cell population as described herein (e.g., in a composition, pharmaceutical composition or medicament as described herein) for use in the treatment of an infectious disease. The present invention also provides at least one immune cell population as described herein (e.g., in a composition, pharmaceutical composition or medicament as described herein) for use in the manufacture of a medicament for treating an infectious disease.

In some embodiments, the infectious disease is a viral infectious disease. As used herein, a “viral infectious disease” may be an infection caused by any virus that causes a disease or pathological condition in the host.

Examples of viral infectious diseases include, but are not limited to, a viral infection caused by an Epstein-Barr virus (EBV); a viral infection caused by a hepatitis A virus, a hepatitis B virus or a hepatitis C virus; a viral infection caused by a herpes simplex type 1 virus, a herpes simplex type 2 virus, or a herpes simplex type 8 virus; a viral infection caused by a cytomegalovirus (CMV); a viral infection caused by a human immunodeficiency virus (HIV); a viral infection caused by an influenza virus; a viral infection caused by a measles or mumps virus; a viral infection caused by a human papillomavirus (HPV); a viral infection caused by a parainfluenza virus; a viral infection caused by a rubella virus; a viral infection caused by a respiratory syncytial virus (RSV); or a viral infection caused by a varicella-zoster virus. In some aspects, a viral infection may lead to or result in the development of cancer in a subject with the viral infection (e.g., HPV infection may cause or be associated with the development of several cancers, including cervical, vulvar, vaginal, penile, anal, and oropharyngeal cancers, and HIV infection may cause the development of Kaposi's sarcoma).

In some embodiments, the infectious disease is a bacterial infectious disease. As used to herein, a “bacterial infectious disease” may be an infection caused by any bacteria that causes a disease or pathological condition in the host.

Examples of bacterial infectious diseases include, but are not limited to, pneumonia, otitis media, sinusitis, bronchitis, tonsillitis and mastoiditis associated with infection by Streptococcus pneumoniae, Haemophilus influenzae, Moraxella catarrhalis, Staphylococcus aureus or genus Peptostreptococcus; pharyngitis, rheumatic fever and glomerulonephritis caused by infection by Streptococcus pyogenes, Group C and G streptococcus, Clostridium diptheriae or Actinobacillus haemolyticum; airway infections associated with infection by Mycoplasma pneumoniae, Legionella pneumophila, Streptococcus pneumoniae, Haemophilus influenzae, or Chlamydia pneumoniae; non-complex skin and soft-tissue infections, boils, osteomyelitis and puerperal fever associated with infection by Staphylococcus aureus, coagulase-positive Staphylococcus (e.g., S. epidermidis, S. hemolyticus, etc.), Streptococcus pyogenes, Streptococcus agalactiae, Streptococcus group C-F (micro-colony Streptococcus), Viridans streptococcus, Corynebacterium minutissimum, genus Clostridium, or Bartonella henselae; uncomplexed acute urinary tract infection; urethritis and cervicitis associated with infection by Staphylococcus saprophyticus or genus Enterococcus; and sexually transmitted disease associated with infection by Chlamydia trachomatis, Haemophilus ducreyi, Treponema pallidum, Ureaplasma urealyticum, or Neiserria gonorrheae; toxic diseases associated with infection by S. aureus (food poisoning and toxic shock syndrome), groups A, B and C streptococci; ulcers associated with infection by Helicobacter pylori; systemic fever syndrome associated with infection by Borrelia recurrentis; Lyme disease associated with infection by Borrelia burgdorferi; conjunctivitis, keratitis and dacryocystitis associated with infection by Chlamydia trachomatis, Neisseria gonorrhoeae, S. aureus, S. pneumoniae, S. pyogenes, H. influenzae or genus Listeria; diffuse Mycobacterium avium syndrome (MAC) disease associated with infection by Mycobacterium avium, Mycobacterium intracellulare; gastroenteritis associated with infection by Campylobacter jejuni; dental infection associated with infection by Viridans streptococcus; persistent cough associated with infection by Bordetella pertussis; gas gangrene associated with infection by Clostridium perfringens or the genus Bacteroides; and atherosclerosis accompanied by infection by Helicobacter pylori or Chlamydia pneumoniae. In certain embodiments, the bacterial infection may be caused by, for example, Escherichia genus, Listeria genus, Salmonella genus, or Staphylococcus genus bacteria.

In some embodiments, the infectious disease is a fungal infectious disease. As used to herein, a “fungal infectious disease” may be an infection caused by any fungus that causes a disease or pathological condition in the host.

Examples of infectious diseases caused by fungus include, but are not limited to, topical, mucosal and/or systemic fungal infections caused by, for example, Candida albicans, Cryptococcus neoformans, Aspergillus flavus, Aspergillus fumigatus, Coccidioides, Paracoccidioides, Histoplasma or Blastomyces. Other exemplary fungal associated disorders include oral thrush, vaginal candidiasis, aspergillosis, candidosis, chromomycosis, coccidioidiocycosis, cryptocococcosis, entomophthoromycosis, epizootic lymphangitis, geotrichosis, histoplasmosis, mucormycosis, mycetoma, North American blastomycosis, oomycosis, paecilimycosis, penicilliosis, rhinosporidiosis, and sprotrichiosis in animals (e.g., humans).

In some embodiments, the infectious disease is a parasistic infectious disease. As used to herein, a “parasitic infectious disease” may be an infection caused by any protozoa, helminths, or ectoparasites that cause a disease or pathological condition in the host.

Examples of protozoa that may be infectious to humans include, but are not limited to, Entamoeba; Giardia, Leishmania balantidium, Plasmodium, and Cryptosporidium.

Examples of helminths that may be infectious to humans include, but are not limited to, Filariasis, Onchocerciasis, Ascariasis, Trichuriasis, Necatoriasis, Trichostrongyliasis, Dracunculiasis, Baylisascaris, Echinococcosis, Hymenolepiasis, Taeniasis, Cysticercosis, Coenurosis, Amphistomiasis, Clonorchiasis, Fascioliasis, Fasciolopsiasis, Opisthorchiasis, Paragonimiasis, Schistosomiasis and Bilharziasis.

Examples of ectoparasites that may be infectious to humans include, but are not limited to, insects (six-legged arthropods) and arachnids (eight-legged arthropods).

### CAR Administration

The CAR-engineered immune cells of the present invention may be administered either alone or as a pharmaceutical composition described herein (e.g., in combination with diluents and/or with other components, including, without limitation, IL-2 or other cytokines or cell populations).

The pharmaceutical compositions of the present invention may be administered to a subject in any suitable manner, including by aerosol inhalation, injection, ingestion, transfusion, implantation or transplantation. In some embodiments, the pharmaceutical compositions described herein may be administered to a subject by parenteral administration. In certain embodiments, the pharmaceutical compositions described herein may be administered to a subject subcutaneously, intradermally, intratumorally, intranodally, intramedullary, intramuscularly, intrasternally, by intravenous (i.v.) injection, by infusion techniques or intraperitoneally. In particular embodiments, the CAR-modified immune cell compositions of the present invention may be administered to a subject by intradermal or subcutaneous injection. In some embodiments, the CAR-modified immune cell compositions of the present invention may be administered by i.v. injection. In some embodiments, the compositions of CAR-modified immune cells may be injected directly into a lymph node, site of infection, site of inflammation or site of tissue or organ rejection. In some embodiments, the compositions of CAR-modified immune cells may be injected directly into the site of the autoimmune and/or inflammatory disease.

In some embodiments, the subject is administered (or is to be administered) with autologous cells.

In some embodiments, the subject is administered (or is to be administered) with allogenic cells.

In some embodiments, the subject may be a mammal. In particular embodiments, the subject may be a human.

Pharmaceutical compositions of the present invention may be administered in a manner appropriate to the disease to be prevented or treated. The quantity and frequency of administration will be determined by such factors as the condition of the subject and the type and severity of the subject's disease, although appropriate dosages may be determined by clinical trials.

When an “effective amount” or “therapeutic amount” is indicated, the precise amount of the compositions of the present invention to be administered may be determined with consideration of individual differences in age, weight, antibody titer, and condition of the subject. It can generally be stated that a pharmaceutical composition comprising the CAR-engineered immune cells as described herein may be administered at a dosage of at least 1×10², 1×10³, 1×10⁴, 1×10⁵, 1×10⁶, 1×10⁷, 1×10⁸ or 1×10⁹ cells/kg body weight or 1×10⁵ to 100×10⁵ cells/kg body weight, including all integer values within those ranges. CAR-engineered immune cell compositions may also be administered multiple times at any of these dosages or any combination thereof. The CAR-engineered immune cells can be administered by using infusion techniques that are commonly known in immunotherapy. The optimal dosage and treatment regimen for a particular subject can readily be determined by monitoring the subject for signs of disease and adjusting the treatment accordingly.

In some embodiments, the CAR-engineered immune cells of the present invention may be administered to a subject in conjunction with (e.g., before, simultaneously or following) any number of relevant treatment modalities, including but not limited to treatment with agents such as antiviral therapy, chemotherapy (i.e., a chemotherapeutic agent), alkylating agents, radiation, immunosuppressive agents, antibodies, immunoablative agents, cytokines, irradiation and anti-infective agents.

In some embodiments, the CAR-engineered immune cells of the present invention may be administered in conjunction with a chemotherapeutic agent. Any chemotherapeutic agent known in the art may be used. Examples of chemotherapeutic agents include, but are not limited to, an anthracycline (e.g., doxorubicin (e.g., liposomal doxorubicin)), a vinca alkaloid (e.g., vinblastine, vincristine, vindesine, or vinorelbine), an alkylating agent (e.g., cyclophosphamide, decarbazine, melphalan, ifosfamide, or temozolomide), an immune cell antibody (e.g., alemtuzamab, gemtuzumab, rituximab, ofatumumab, tositumomab, or brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

Other chemotherapeutic agents considered for use in combination therapies of the invention include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

The CAR-engineered immune cells of the present invention may be administered to the subject before, after, or concomitant with the chemotherapeutic agent.

In some embodiments, the CAR-engineered immune cells of the present invention may be administered in conjunction with an alkylating agent. Any alkylating agent known in the art may be used. Examples of alkylating agents include, without limitation, nitrogen mustards, ethylenimine derivatives, alkyl sulfonates, nitrosoureas and triazenes, uracil mustard (Aminouracil Mustard®, Chlorethaminacil®, Demethyldopan®, Desmethyldopan®, Haemanthamine®, Nordopan®, Uracil nitrogen mustard®, Uracillost®, Uracilmostaza®, Uramustin®, and Uramustine®), chlormethine (Mustargen®), cyclophosphamide (Cytoxan®, Neosar®, Clafen®, Endoxan®, Procytox®, and Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, and Hexastat®), triethylenethiophosphoramine, Temozolomide (Temodar®), thiotepa (Thioplex®), busulfan (Busilvex® and Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, and Thioplex®); Cyclophosphamide (Endoxan®, Cytoxan®, Neosar®, Procytox®, and Revimmune®); and Bendamustine HCl (Treanda®).

The CAR-engineered immune cells of the present invention may be administered to the subject before, after, or concomitant with the alkylating agent.

In some embodiments, the CAR-engineered immune cells of the present invention may be administered in conjunction with an immunosuppressant agent. Any immunosuppressant agent known in the art may be used. Examples of immunosuppressant agents include, but are not limited to, cyclosporine, azathioprine, methotrexate, methoxsalen, rapamycin, mycophenolate mofetil, mycophenolic acid, rituximab, sirolimus, basiliximab, daclizumab, muromonab-CD3, tacrolimus, glucorticosteroids, adrenocortical steroids such as prednisone and prednisolone, and any combination thereof.

The CAR-engineered immune cells of the present invention may be administered to the subject before, after, or concomitant with the immunosuppressant agent.

The CAR-engineered immune cells of the present invention and/or the immunosuppressant agent(s) may be administered to the subject after transplantation. Alternatively, or in addition, the CAR-engineered immune cells of the present invention and/or the immunosuppressant agent(s) may be administered to the subject before transplantation. In some embodiments, the CAR-engineered immune cells of the present invention and/or the immunosuppressant agent may be administered to the subject during transplantation surgery.

In some embodiments, the administration of CAR-engineered immune cells to the subject is carried out once immunosuppressive therapy has been initiated.

In some embodiments, the method is carried out more than once, e.g., to monitor the transplant recipient over time, and, if applicable, in different immunosuppressive therapy regimes.

In some embodiments, immunosuppressive therapy is reduced if the transplant recipient is predicted to be tolerant of the transplant. In some embodiments, no immunosuppressive therapy is prescribed, e.g., immunosuppressive therapy is ceased, if the transplant recipient is predicted to be tolerant of the transplant.

The CAR-engineered immune cells of the present invention may be administered following a diagnosis of transplant organ or tissue rejection followed by doses of both the CAR-engineered immune cells of the invention and immunosuppressant agent(s) until symptoms of organ or tissue rejection subside.

In a further embodiment, the CAR-engineered immune cell compositions of the present invention may be administered to a subject in conjunction with (e.g., before, simultaneously with, or following) bone marrow transplantation.

In some embodiments, the CAR-engineered immune cells of the present invention may be administered following B cell ablative therapy such as agents that react with CD20, e.g., rituximab. For example, in some embodiments, subjects may undergo standard treatment with high dose chemotherapy followed by peripheral blood stem cell transplantation. In certain embodiments, following the transplant, subjects may receive an infusion of the expanded CAR-engineered immune cells of the present invention. In certain embodiments, expanded CAR-engineered immune cells may be administered before or following surgery.

In some embodiments, the CAR-engineered immune cells of the present invention may be administered in conjunction with an anti-infective agent. Any anti-infective agent known in the art may be used. Examples of anti-infective agents include, but are not limited to, amebicides, aminoglycosides, anthelmintics, antiparasitics, antifungals (azole antifungals, echinocandins, miscellaneous antifungals, and polyenes), antimalarial agents (antimalarial combinations, antimalarial quinolines, and miscellaneous antimalarials), antituberculosis agents (aminosalicylates, antituberculosis combinations, diarylquinolines, hydrazide derivatives, miscellaneous antituberculosis agents, nicotinic acid derivatives, rifamycin derivatives, and streptomyces derivatives), antiviral agents (adamantane antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors (NRTIs), protease inhibitors, and purine nucleosides), carbapenems, carbapenems/beta-lactamase inhibitors, cephalosporins (cephalosporins/beta-lactamase inhibitors, first generation cephalosporins, fourth generation cephalosporins, next generation cephalosporins, second generation cephalosporins, and third generation cephalosporins), antibiotics, glycopeptide antibiotics, glycylcyclines, leprostatics, lincomycin derivatives, macrolide derivatives (ketolides and macrolides), miscellaneous antibiotics, oxazolidinone antibiotics, penicillins (aminopenicillins, antipseudomonal penicillins, beta-lactamase inhibitors, natural penicillins, penicillinase, and resistant penicillins), quinolones, sulfonamides, tetracyclines, and urinary anti-infectives.

In some embodiments, the subject (e.g., human) receives an initial administration of an immune cell or population of the invention, and one or more subsequent administrations, wherein the one or more subsequent administrations are administered less than 15 days, e.g., 14, 13, 12, 11, 10, 9, 8, 7, 6, 5, 4, 3, or 2 days after the previous administration.

In some embodiments, a therapeutically effective amount of immune cells of the invention is administered or is to be administered to the subject.

In some embodiments, the number of immune cells of the immune cell population of the invention administered to the subject is at least of 10², 10³, 10⁴, 10⁵, 10⁶, 10⁷, 10⁸ or 10⁹ cells.

In some embodiments, the number of immune cells of the immune cell population of the invention administered to the subject ranges from about 10² to about 10⁹, from about 10³ to about 10⁸, from about 10⁴ to about 10⁷, or from about 10⁵ to about 10⁶ cells.

In some embodiments, the number of immune cells of the immune cell population of the invention administered to the subject ranges from about 10² to about 10⁹, from about 10² to 10⁸, from about 10² to 10⁷, from about 10² to 10⁶, from about 10² to 10⁵, from about 10² to 10⁴, or from about 10² to 10³ cells. In some embodiments, the number of immune cells of the immune population of the invention administrated to the subject is about 10², about 10³, about 10⁴, about 10⁵, about 10⁶, about 10⁷, about 10⁸, or about 10⁹ cells.

In some embodiments, the number of immune cells of the immune cell population of the invention administered to the subject is at least 10², 10³, 10⁴, 10⁵, 10⁶, 10⁷, 10⁸ or 10⁹ cells/kg body weight.

In some embodiments, the number of immune cells of the immune cell population of the invention administered to the subject ranges from about 10² to 10⁹ cells/kg body weight or 10³ to 10⁸ cells/kg body weight, including all integer values within those ranges.

In some embodiments, the subject receives more than one administration of the immune cell population of the invention per week, e.g., 2, 3, or 4 administrations of a Treg cell population of the invention administered per week to the subject.

In some embodiments, the Treg cell population is administered to the subject in need thereof in combination with an active agent. According to some embodiments, the immune cell population is administered before, at the same time as, or after the administration of an active agent.

In some embodiments, it may be desirable to administer activated immune cells of the invention to a subject and then subsequently redraw blood (or have an apheresis performed), activate immune cells therefrom according to the present invention, and reinfuse the subject with these activated and expanded immune cells. This process can be carried out multiple times every few weeks. In certain embodiments, immune cells can be activated from blood draws of from 10 cc to 400 cc. In certain embodiments, immune cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of immune cells.

It is understood that the CARs, cell populations, and compositions described herein may be used in a method of treatment as described herein, may be for use as a medicament as described herein, may be for use in a treatment as described herein, and/or may be for use in the manufacture of a medicament for a treatment as described herein.

### Articles of Manufacture and Kits

The present invention also provides articles of manufacture comprising any of the nucleic acids, vectors, cell populations, or compositions described herein, as well as methods for manufacturing said articles.

Further, the present invention provides kits comprising in a suitable container any of the nucleic acids, vectors, cell populations, or compositions described herein.

### Embodiments

Particular embodiments of the invention are listed below:
1. A chimeric antigen receptor (CAR) comprising:
   at least one extracellular binding domain,
   optionally at least one extracellular hinge domain,
   at least one transmembrane domain,
   at least one intracellular domain,
   wherein the at least one intracellular domain comprises optionally at least one costimulatory intracellular signaling domain and at least one primary intracellular signaling domain, and
   wherein
   the at least one transmembrane domain is a human TNFR2 transmembrane domain or a fragment or variant thereof or any transmembrane domain or a fragment or variant thereof or a combination thereof, and/or
   the at least one costimulatory intracellular signaling domain is a human TNFR2 costimulatory intracellular signaling domain or a fragment or variant thereof or any costimulatory intracellular signaling domain or a fragment or variant thereof or a combination thereof, and
   wherein at least one of the transmembrane domain and costimulatory intracellular signaling domain is TNFR2 transmembrane domain or a fragment or variant thereof or TNFR2 costimulatory intracellular signaling domain or a fragment or variant thereof
2. The CAR according to embodiment 1, wherein said human TNFR2 transmembrane domain comprises at least 2 amino acids from the sequence SEQ ID NO: 22 or from a sequence having at least about 70% identity with SEQ ID NO: 22, preferably at least 2 contiguous amino acids from SEQ ID NO: 22 or from a sequence having at least about 70% identity with SEQ ID: 22.
3. The CAR according to embodiment 1 or embodiment 2, wherein said human TNFR2 transmembrane domain is combined with at least one other transmembrane domain.
4. The CAR according to any one of embodiments s 1 to 3, wherein said human TNFR2 costimulatory intracellular signaling domain comprises at least 2 amino acids from the sequence SEQ ID NO: 34 or from a sequence having at least about 70% identity with SEQ ID NO: 34, preferably at least 2 contiguous amino acids from SEQ ID NO: 34 or from a sequence having at least about 70% identity with SEQ ID: 34.
5. The CAR according to any one of embodiments s 1 to 4, wherein said TNFR2 costimulatory intracellular signaling domain is combined with least one other costimulatory intracellular signaling domain.
6. The CAR according to any one of embodiments 1 to 5, wherein the primary intracellular signaling domain comprises an immune cell primary intracellular signaling domain, preferably a T cell primary intracellular signaling domain of human CD3, more preferably a T cell primary intracellular signaling domain of human CD3 zeta having a sequence SEQ ID NO: 28, 29, 30 or 31 or a sequence having at least about 70% identity with SEQ ID: 28, 29, 30 or 31.
7. The CAR according to any one of embodiments 1 to 6, wherein the hinge domain is a hinge region of human CD8, preferably SEQ ID NO: 14 or a sequence having at least about 70% identity with SEQ ID NO: 14.
8. The CAR according to any one of embodiments 1 to 7, wherein the CAR comprises at least one TNFR2 transmembrane domain and at least one intracellular domain, wherein said intracellular domain comprises optionally at least one costimulatory intracellular signaling domain and at least one immune cell primary intracellular signaling domain.
9. The CAR according to any one of embodiments 1 to 7, wherein the CAR comprises at least one transmembrane domain and at least one intracellular domain, wherein said intracellular domain comprises at least one TNFR2 costimulatory intracellular signaling domain and at least one immune cell primary intracellular signaling domain.
10. A nucleic acid sequence encoding the CAR according to any one of embodiments 1 to 9.
11. A vector comprising the nucleic acid sequence according to embodiment 10.
12. An immune cell population comprising the nucleic acid sequence of embodiment 10, or the vector of embodiment 11, or expressing a CAR according to any one of embodiments 1 to 9.
13. The immune cell population according to embodiment 12, wherein said immune cell population is selected from the group comprising T cells, natural killer (NK) cells, γδ T cells, double negative (DN) cells, regulatory immune cells, regulatory T cells, effector immune cells, effector T cells, B cells and myeloid-derived cells, and any combination thereof
14. A composition comprising at least one cell population according to any one of embodiments 12 to 13.
15. The composition according to embodiment 14, being a pharmaceutical composition and further comprises at least one pharmaceutically acceptable excipient.
16. A method for treating a disease or disorder in a subject in need thereof, comprising administering to a subject at least one immune cell population according to embodiments 12 or 13 or a composition according to embodiments 14 or 15.
17. The method according to embodiment 16, wherein said method is a cell therapy method.
18. The method according to embodiments 16 or 17, wherein said disease or disorder include inflammatory diseases, autoimmune diseases, allergic diseases, organ transplantation conditions, cancers and infectious diseases.
19. The method according to any one of embodiments 16 to 18, wherein the immune cell population is a regulatory immune cell population, or a Treg cell population and wherein said disease or disorder is an inflammatory disease, an autoimmune disease, an allergic disease, or an organ transplantation condition, preferably selected from graft rejection and graft-versus-host disease.
20. The method according to any one of embodiments 16 to 18, wherein the immune cell population is a T effector cell population, a NK cell population or a γδ cell population and wherein said disease or disorder is a cancer or an infectious disease.

The present invention relates to a chimeric antigen receptor (CAR) comprising:
at least one extracellular binding domain,
optionally at least one extracellular hinge domain,
at least one transmembrane domain (e.g., a human TNFR2 transmembrane domain or a fragment or variant thereof, any transmembrane domain or a fragment or variant thereof, or any combination thereof), and
at least one intracellular domain (comprising at least one primary intracellular signaling domain and optionally comprising at least one costimulatory intracellular signaling domain or a fragment or variant thereof, wherein the at least one costimulatory intracellular signaling domain is a human TNFR2 costimulatory intracellular signaling domain or a fragment or variant thereof, any costimulatory intracellular signaling domain or a fragment or variant thereof, or any combination thereof),
   wherein the transmembrane domain is a TNFR2 transmembrane domain or a fragment or variant thereof, and/or the costimulatory intracellular signaling domain is a TNFR2 costimulatory intracellular signaling domain or a fragment or variant thereof.

In some embodiments, said human TNFR2 transmembrane domain or fragment or variant thereof comprises at least 2 amino acids (e.g., at least 2 contiguous amino acids) from the sequence of SEQ ID NO: 22 or from a sequence having at least about 70% identity with SEQ ID NO: 22.

In some embodiments, said human TNFR2 transmembrane domain or fragment or variant thereof is combined with at least one other transmembrane domain or fragment or variant thereof.

In some embodiments, said human TNFR2 costimulatory intracellular signaling domain or variant or fragment thereof comprises at least 2 amino acids (e.g., at least 2 contiguous amino acids) from the sequence of SEQ ID NO: 34 or from a sequence having at least about 70% identity with SEQ ID NO: 34.

In some embodiments, said TNFR2 costimulatory intracellular signaling domain or variant or fragment thereof is combined with least one other costimulatory intracellular signaling domain or fragment or variant thereof.

In some embodiments, the primary intracellular signaling domain comprises an immune cell primary intracellular signaling domain. In certain embodiments, the primary intracellular signaling domain is a T cell primary intracellular signaling domain of human CD3. In particular embodiments, the primary intracellular signaling domain is a T cell primary intracellular signaling domain of human CD3 zeta having a sequence of SEQ ID NO: 28, 29, 30 or 31 or a sequence having at least about 70% identity with SEQ ID: 28, 29, 30 or 31.

In some embodiments, the hinge domain is a hinge region of human CD8, preferably SEQ ID NO: 14 or a sequence having at least about 70% identity with SEQ ID NO: 14.

In some embodiments, the CAR comprises at least one TNFR2 transmembrane domain or a fragment or variant thereof, and at least one intracellular domain, wherein said intracellular domain comprises at least one immune cell primary intracellular signaling domain and optionally comprises at least one costimulatory intracellular signaling domain.

In some embodiments, the CAR comprises at least one transmembrane domain and at least one intracellular domain, wherein said intracellular domain comprises at least one TNFR2 costimulatory intracellular signaling domain or a fragment or variant thereof and at least one immune cell primary intracellular signaling domain.

The present invention further relates to a nucleic acid sequence encoding a CAR according to the present invention.

The present invention further relates to a vector comprising the nucleic acid sequence according to the present invention.

The present invention further relates to an immune cell population comprising the nucleic acid sequence according to the present invention, or the vector according to the present invention, or expressing a CAR according to the present invention. In some embodiments, the immune cell population is selected from the group consisting of T cells, natural killer (NK) cells, γδ T cells, double negative (DN) cells, regulatory immune cells, regulatory T cells, effector immune cells, effector T cells, B cells and myeloid-derived cells, and any combination thereof.

The present invention further relates to a composition comprising at least one cell population according to the present invention. In some embodiments, the composition is a pharmaceutical composition and further comprises at least one pharmaceutically acceptable excipient.

The present invention further relates to a method for treating a disease or disorder in a subject in need thereof, comprising administering to a subject at least one cell population or composition according to the present invention. In some embodiments, the method is a cell therapy method. In some embodiments, said diseases or disorders include inflammatory diseases, autoimmune diseases, allergic diseases, organ transplantation conditions, cancers and infectious diseases. In some embodiments, the cell population is a regulatory immune cell population or a Treg cell population, and said disease or disorder is an inflammatory disease, an autoimmune disease, an allergic disease, or an organ transplantation condition (e.g., graft rejection or graft-versus-host disease). In some embodiments, the immune cell population is a T effector cell population, a NK cell population or a γδ cell population and said disease or disorder is a cancer or an infectious disease.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### EXAMPLES

The present invention is further illustrated by the following examples.

### Example 1: TNFR2-Derived CAR-Tregs

### Materials and Methods

### PBMC Isolation

Blood cells of anonymous healthy donors were collected by the Etablissement Français du Sang (EFS) following EFS guidelines and informed consent of the donors. The day after blood collection, peripheral blood mononuclear cells (PBMC) were isolated from buffy coats by Ficoll gradient centrifugation, which enables removal of unwanted fractions of blood product such as granulocytes, platelets and remaining red blood cell contaminants. Cells of interest were then isolated as described below.

### FoxP3 Treg Isolation

CD4⁺CD25⁺CD127^{low} Tregs were isolated using the Human CD4⁺CD127^{low}CD25⁺ Regulatory T Cell Isolation Kit (StemCell), following manufacturer's instructions. Briefly, CD25⁺ cells were first isolated from 400-500×10⁶ PBMC by column-free, immunomagnetic positive selection using EasySep™ Releasable RapidSpheres™. Then, bound magnetic particles were removed from the EasySep™-isolated CD25⁺ cells, and unwanted non-Tregs were targeted for depletion. The final isolated fraction contained highly purified CD4⁺CD127^{low}CD25⁺ cells that express high levels of FoxP3 and were immediately used for downstream applications. Autologous B cells isolation

Autologous CD19⁺CD20⁺ B cells were isolated using the Human B Cell Isolation Kit (StemCell) following manufacturer's instructions. Briefly, CD19⁺CD20⁺ B cells were isolated from 200×10⁶ PBMC by immunomagnetic negative selection. After isolation, cells were immediately frozen for further use as CD19⁺CD20⁺ presenting cells.

CD4⁻CD25⁻ Conventional T Cells Isolation

CD4⁺CD25⁻ T cells were isolated by carrying out the optional protocol of the Regulatory T Cell Isolation Kit (StemCell) allowing the isolation of CD4⁺CD25⁻ T cells for use in functional studies in parallel to Tregs.

### Activation and Culture of Isolated Tregs

Isolated Treg cells were activated and cultured for 9 days. Briefly, at day 0, Treg cells (0.5×10⁶) were cultured into 24 wells plate (Costar) in Xvivo15 serum-free medium containing human transferrin (OZYME) and supplemented with 1000 U/mL IL-2 (Euromedex) and 100 nM rapamycin (Sigma-Aldrich). Polyclonal activation was performed with anti-CD3/anti-CD28 Dynabeads from Life Technology (0.5×10⁶ beads per well). At days 2, 4 or 5, and 7 or 8, cells were fed with fresh culture medium supplemented with 1000 U/mL IL-2. Finally, at day 8 or 9, cells were recovered, counted and reactivated.

### Transduction Protocol

Tregs were transduced with a chimeric antigen receptor (see below) 2 days after their activation. Briefly, transduction was carried out by loading 0.5×10⁷ Transduction Units (TU) per mL to each well. After 6 hours at 37° C., viral particles were removed by washout. The plates were then incubated at 37° C. with 5% CO₂. After five days, the transduction efficiency was analyzed by measuring the percentage of GFP positive cells in flow cytometry.

### Quantification of CAR Expression

The quantification of cell surface CAR expression was performed by labelling the CAR with APC-conjugated protein L or an APC-conjugated anti-hemagglutinin (HA) antibody for HA-tagged CARs, and analyzed using flow cytometry.

The quantification of total CAR expression was performed by Western blot analysis. Briefly, 5×10⁴ transduced Tregs or untransduced (“Blank”) Tregs were lysed in RIPA-buffer, subjected to SDS-PAGE in denaturating conditions, and blotted on a PVDF-membrane. Then, the CAR-expressed CD3ζ as well as the endogenous CD3ζ were stained with CD3ζ specific antibody (anti-CD247, BD Pharmingen). Finally, the membrane was washed and reprobed with β-actin antibody as loading control. Image J software was used to quantify band intensity.

### CAR Constructs Used for Transduction

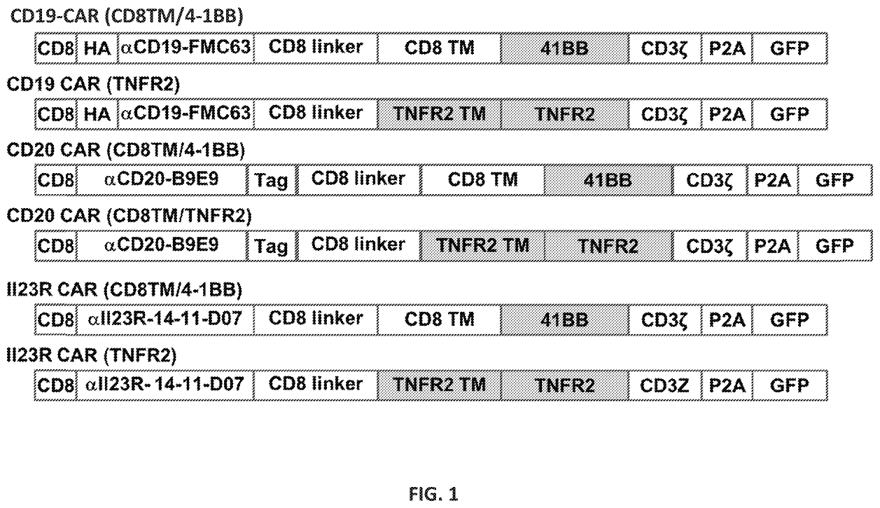

CARs composed of the TNFR2, TNFR1, or CD8 transmembrane (TM) and costimulatory intracellular signaling domains in tandem with CD3ζ and associated with a prototypical scFv directed against CD19 (FMC63), CD20 (B9E9), and/or IL-23R (14-11-D07; PCT Patent Publication WO 2016/184570; SEQ ID NO: 65) were designed. The constructs used in this study are listed and described in Table 1 and FIG. 1

TABLE 1
CAR constructs

| | | | costimulatory | |
|---|---|---|---|---|
| | | | intracellular | |
| | | | signaling | |
| Name of the CAR | scFv | TM | domain | CD3ζ |
| CD19-CAR (CD8TM/4- | FMC63 | CD8 | 4-1BB | YES |
| 1BB) | | | | |
| CD19-CAR (TNFR2) | FMC63 | TNFR2 | TNFR2 | YES |
| CD20-CAR (CD8TM/4- | B9E9 | CD8 | 4-1BB | YES |
| 1BB) | | | | |
| CD20-CAR (TNFR2) | B9E9 | TNFR2 | TNFR2 | YES |
| CD20-CAR (TNFR1) | B9E9 | TNFR1 | TNFR1 | YES |
| IL-23R-CAR (CD8TM/4- | 14-11-D07 | CD8 | 4-1BB | YES |
| 1BB) | | | | |
| IL-23R-CAR (TNFR2) | 14-11-D07 | TNFR2 | TNFR2 | YES |

More precisely, the anti-CD19 CARs are composed of the human CD8 leader sequence (aa1-22), a Hemagglutinin tag, the FMC63 scFv directed against human CD19, and a linker derived from human CD8 alpha (aa138-182).

Following the linker, the CD19-CAR (CD8TM/4-1BB) construct is composed of the transmembrane (TM) domain of human CD8 alpha (aa183-206) and the costimulatory intracellular signaling domain of human 4-1BB (aa214-255) and CD3 zeta (aa52-164); while the CD19-CAR (TNFR2) construct is composed of the TM and intracellular domain of human TNFR2 (aa258-461) and CD3 zeta.

The anti-CD20 CARs are composed of the human CD8 leader sequence (aa1-22), the B9E9 scFv directed against human CD20, a streptavidin tag, and a linker derived from human CD8 alpha (aa138-182).

Following the linker, the CD20-CAR (CD8TM/4-1BB) construct is composed of the TM domain of human CD8 alpha (aa183-206) and the costimulatory intracellular signaling of human 4-1BB (aa214-255) and CD3 zeta (aa52-164); while:
the CD20-CAR (TNFR2) construct is composed of the TM and costimulatory intracellular signaling domains of human TNFR2 (aa258-461) and CD3 zeta; and
the CD20-CAR (TNFR1) is composed of the TM and costimulatory intracellular signaling domains of human TNFR1 (aa212-455) and CD3 zeta.

The anti-IL-23R CARs are composed of the human CD8 leader sequence (aa1-22), an scFv directed against human IL-23R, and a linker derived from human CD8 alpha (aa138-182).

Following the linker, the IL-23R-CAR (CD8TM/4-1BB) construct is composed of the transmembrane domain of human CD8 alpha (aa183-206) and the costimulatory intracellular signaling domain of human 4-1BB (aa214-255) and CD3 zeta (aa52-164); while the IL-23R-CAR (TNFR2) construct is composed of the transmembrane and costimulatory intracellular signaling domain of human TNFR2 (aa258-461) and CD3 zeta.

All the CAR constructs are cloned in phase with a P2A linker and the open reading frame of enhanced green fluorescent protein (GFP).

### Phenotype Analysis of Transduced Treg

At day 9 of the culture, Treg phenotype was analyzed by flow cytometry, to ensure that the transduction procedure did not impact Treg status. Markers used for this analysis are listed in Table 2.

TABLE 2
Materials and reagents

| Reagents | Manufacturer | Cat. No. |
|---|---|---|
| CD4 VioGreen | Miltenyi | 130-096-900 |
| Helios eF450 (HamIgG) | eBioscience | 48-9883-42 |
| CD25 PE | Miltenyi | 130-109-020 |
| CD152 (CTLA-4) PE/Cy7 (mIgG2a) | Biolegend | 369614 |
| FoxP3 AF647 (mIgG1; 2 μl) | BD | 560045 |
| CD127-APC-Vio770 | Miltenyi | 130-109-438 |

### Activation Assay of CARs

CAR activation was evaluated either in the Jurkat-Lucia-NFAT cell line (Invivogen) or in human primary Tregs. For the reporter cell line, following CAR engagement with CD19⁺ Daudi cells (1:1 ratio), the NFAT activation was evaluated by monitoring Lucia luciferase activity (see manufacturer's instructions) and using a GloMax luminometer (Promega).

For Tregs, the activation assay was performed at day 9 of the culture. Briefly, 0.05×10⁶ Tregs were seeded in a 96 U bottom plate alone or in the presence of anti-CD28/anti-CD3 coated beads (in a 1:1 Treg to beads ratio), or in the presence of freshly thawed autologous B cells or CD19⁺ Daudi B cells (in a 1:1 Treg to B cell ratio) in a 200 μL final volume. After 24 h at 37° C., 5% CO2, cells were stained for CD4 and CD69 and then analyzed using flow cytometry. The monitoring of CD69 spontaneous expression by CAR Treg cells as compared to untransduced Treg cells allows determination of tonic signaling intensity.

### Suppression Assay of T Cell Proliferation

The suppression assay was performed at day 9 of the culture. Briefly, Tregs were recovered, counted and activated either through the TCR using anti-CD28/anti-CD3 coated beads (in a 1:1 Treg to beads ratio), or through the CAR using freshly thawed autologous B cells (in a 1:1 Treg to beads ratio), or kept without activation to evaluate their spontaneous suppressive activity.

In parallel, allogeneic Tconvs (conventional T cells) were thawed, stained with Dye 450 and activated with anti-CD28/anti-CD3 coated beads (in a 3:1 Tconv to beads ratio). The day after, beads were removed from Tconvs before their co-culture with unactivated or activated Tregs (untransduced or transduced).

At day 3, cells were harvested, and proliferation of Tconvs was assessed by flow cytometry through determination of Dye 450 dilution. The percentage of inhibition of Tconv proliferation was calculated as followed:

$$100 - \frac{\% of Tconv proliferation in presence of CAR \text{-} Treg \times 100}{\% of Tconv proliferation in absence of CAR \text{-} Treg}$$

### Results

### Transduction Efficiency and CAR Expression at Cell Surface

Transduction efficiency was determined by assessing the percentage of GFP positive cells, and CAR expression was monitored by assessing recombinant protein L, an immunoglobulin kappa light chain-binding protein for CD20-CAR (CD8TM/4-1BB and TNFR2) and IL-23R CAR (CD8TM/4-1BB and TNFR2), or an antibody directed against HA Tag for CD19-CAR (CD8TM/4-1BB and TNFR2).

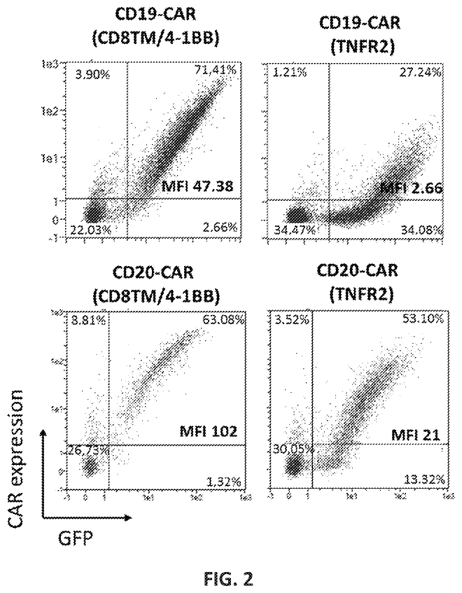

Results for the percentage of transduction efficiency and the percentage of transduced cells that expressed the CAR at cell surface are shown in Table 3 (FIG. 2 shows an example of raw data), as an overview of all donors tested in this Example (n=5).

TABLE 3
Transduction efficiency and CAR expression at cell surface

| | CD19-CAR | CD19- | CD20-CAR | CD20- | IL-23R-CAR | IL-23R- |
|---|---|---|---|---|---|---|
| CAR | (CD8TM/ | CAR | (CD8TM/ | CAR | (CD8TM/ | CAR |
| constructs | 4-1BB) | (TNFR2) | 4-1BB) | (TNFR2) | 4-1BB) | (TNFR2) |
| TU/mL for | 5 × 10⁶ | | 2 × 10⁶ | | 2 × 10⁶ | 5 × 10⁶ |
| transduction | | | | | | |
| % of | 71.4 ± 2.4 | 65.3 ± 2.0 | 62 ± 2.7 | 66 ± 0.1 | 54.4 ± 7.7 | 47.2 ± 4.1 |
| transduction | | | | | | |
| efficacy | | | | | | |
| % of CAR | 96.2 ± 0.15 | 46.8 ± 2.5 | 98 ± 0.1 | 74.3 ± 8 | 97.2 ± 1.4 | 74.1 ± 8.9 |
| expression at | | | | | | |
| cell surface | | | | | | |
| MFI of the | 47.9 ± 0.3 | 3.2 ± 0.4 | 92.5 ± 13.5 | 16.5 ± 6.4 | 191.1 ± 53.2 | 20.5 ± 3.4 |
| CAR | | | | | | |

As shown in FIG. 2 and/or Table 3, CD19-CAR (CD8TM/4-1BB), CD20-CAR (CD8TM/4-1BB), and IL-23R-CAR (CD8TM/4-1BB) transduced cells harbored more than 95% of the CAR at the cell-surface, while CD19-CAR (TNFR2), CD20-CAR (TNFR2), and IL-23R-CAR (TNFR2) transduced cells expressed 46% to 75% of the CAR at the cell surface. Furthermore, the mean fluorescence intensity (MFI) representing the number of CARs per cell was decreased about 15-fold for CD19-CAR (TNFR2), about 6-fold for CD20-CAR (TNFR2), and about 9-fold for IL-23R-CAR (TNFR2). This strong decrease of the CAR expression at the cell surface was not the consequence of lower transduction efficiency since GFP expression was comparable in all of the experimental conditions.

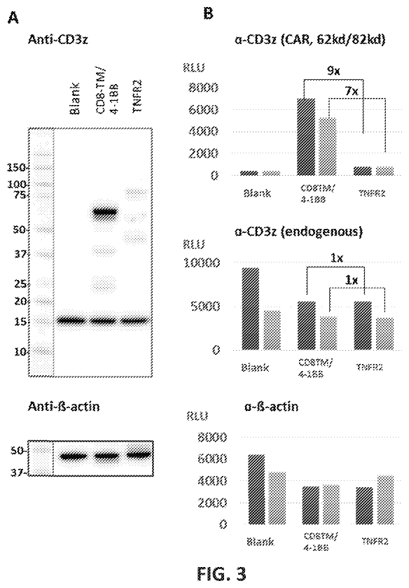

Moreover, as shown in FIG. 3, Panel A, cells transduced with CD20-CAR (CD8TM/4-1BB) expressed a 62 kD protein corresponding to the CAR after staining with CD3ζ antibody and a 82 kD protein for cells transduced with CD20-CAR (TNFR2), while untransduced cells were only labeled with a band at 16 kD corresponding to the endogenous CD3ζ. Interestingly, quantitation of band intensity revealed a lower expression of CD20-CAR (TNFR2) compared to CD20-CAR (CD8TM/4-1BB) (FIG. 3, Panel B) as observed using flow cytometry (FIG. 2).

In summary, the results demonstrated that the TNFR2 intracellular domain and TNFR2 transmembrane domain surprisingly led to a reduced global expression of the CAR, especially at the cell surface.

### CAR-Specific Activation

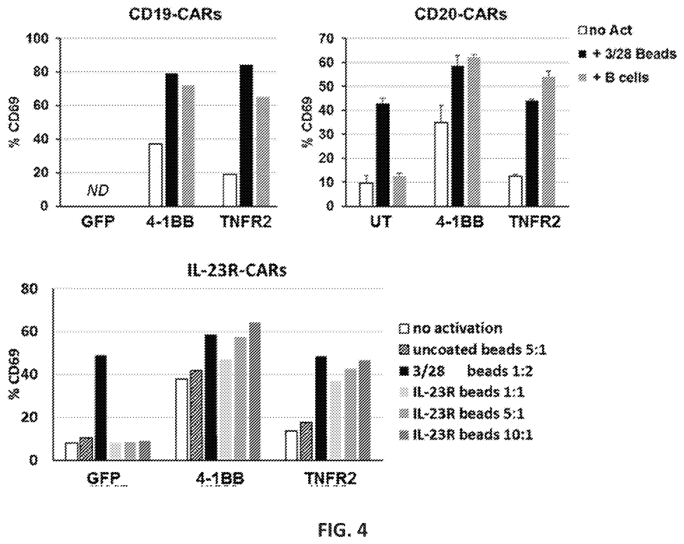

A complete reduction of antigen-independent tonic signaling is observed in Treg cells transduced with CARs comprising TNFR2 domains for the three different scFv targets (CD19, CD20, and IL-23R) as compared to classical 4-1BB/CD3 constructs (FIG. 4, Panels A-C). Furthermore, despite a strong reduction in TNFR2-derived CAR expression (CD19, CD20, or IL-23R) at the cell surface (Table 3), the CAR-specific activation is maintained.

Thus, these results demonstrate that the presence of a TNFR2 transmembrane and TNFR2 intracellular domain surprisingly led to a strong reduction of the activation background in CAR Treg cells and consequently increased the ratio between CAR specific activation and ligand-independent tonic signaling. This phenomenon is independent of the scFv of interest since three different scFvs were tested and behaved in the same manner (CD19, CD20, and IL-23R).

### CAR-Mediated Suppressive Activity

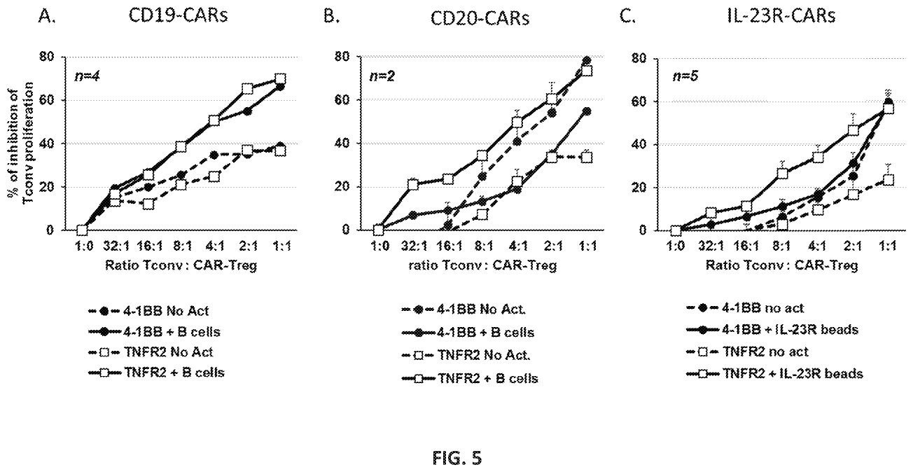

For the CD19-CAR construct harboring TNFR2-derived domains, a CAR-specific triggering of suppressive activity comparable to that of the 4-1BB derived construct was observed even with a 15-fold decrease of the CAR expression at the cell surface (FIG. 5, Panel A).

Most interestingly, in the case of the CD20-CAR and IL-23R-CAR constructs, whereas the spontaneous suppressive activity of the 4-1BB derived construct was too strong to highlight a CAR-mediated suppressive activity, the TNFR2-derived CAR construct provided a strongly reduced background of suppressive activity that for the first time allowed the observation of a specific CAR-mediated suppressive activity (FIG. 5, Panels B and 5C).

In conclusion, the reduction of ligand-independent tonic signaling in a TNFR2/CD3 derived CAR allowed observation for the first time of CD20-CAR- and IL-23R-CAR-mediated suppressive activity, while the same scFvs fused to a 4-1BB/CD3ζ domain had no observable activity over background.

### Example 2: Comparison of TNFR2 Derived CD19-CARs

### Materials and Methods

Except for CAR constructs, the Materials and Methods are the same as those described in Example 1. Here, two types of cells were transduced with CAR constructs: human Tregs and Jurkat-Lucia-NFAT cells.

### CAR Constructs Used for Transduction

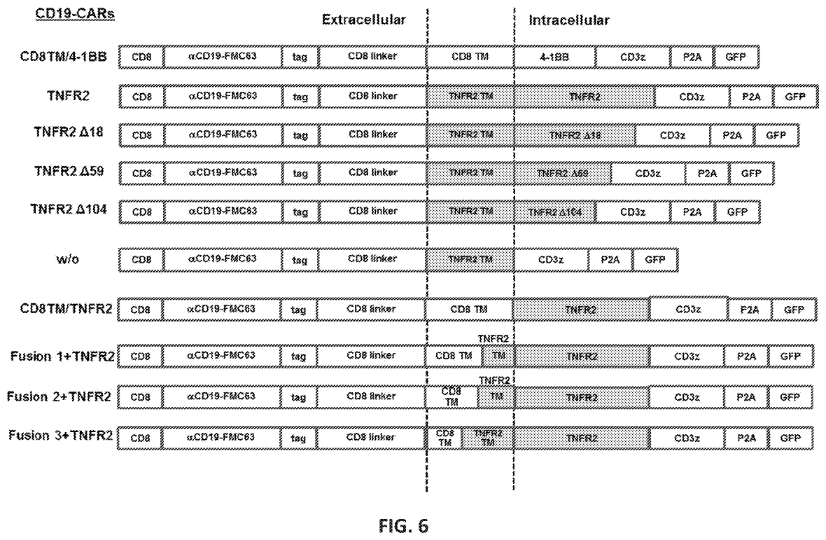

The CAR constructs used in this study are listed and described in Table 4 and FIG. 6.

TABLE 4
CD19-CARs constructs

| | | | costimulatory | |
|---|---|---|---|---|
| | | | intracellular | |
| | | | signaling | |
| Name of the CAR | scFv | TM | domain | CD3ζ |
| CD19-CAR (CD8TM/4-1BB) | FMC63 | CD8 | 4-1BB | YES |
| CD19-CAR (TNFR2) | FMC63 | TNFR2 | TNFR2 | YES |
| CD19-CAR (TNFR2 Δ18) | FMC63 | TNFR2 | TNFR2 Δ18 | YES |
| CD19-CAR (TNFR2 Δ59) | FMC63 | TNFR2 | TNFR2 Δ59 | YES |
| CD19-CAR (TNFR2 Δ104) | FMC63 | TNFR2 | TNFR2 Δ104 | YES |
| CD19-CAR (TNFR2 Δ151) | FMC63 | TNFR2 | TNFR2 Δ151 | YES |
| CD19-CAR (without) | FMC63 | TNFR2 | without | YES |
| CD19-CAR (CD8TM/TNFR2) | FMC63 | CD8 | TNFR2 | YES |
| CD19-CAR (Fusion 1 + TNFR2) | FMC63 | Fusion 1 CD8/TNFR2 | TNFR2 | YES |
| CD19-CAR (Fusion 2 + TNFR2) | FMC63 | Fusion 2 CD8/TNFR2 | TNFR2 | YES |
| CD19-CAR (Fusion 3 + TNFR2) | FMC63 | Fusion 3 CD8/TNFR2 | TNFR2 | YES |

The anti-CD19 CARs are composed of the human CD8 leader sequence (aa1-22), the FMC63 scFv directed against human CD19, a streptavidin tag, and a linker derived from human CD8 alpha (aa138-182).

In some cases, the TNFR2 costimulatory intracellular signaling domain is a fragment of TNFR2 domain where 18, 59, 104, or 151 amino acid residues have been removed at the C-terminus (Δ18, Δ59, Δ104, or Δ151, respectively). The TNFR2 signaling domain can be subdivided into 5 domains, each of which is important for interaction with different signaling molecules to trigger a variety of signaling pathways. Additionally, certain domains are described to modulate the endocytosis of TNFR2 (Ji et al., Arterioscler Thromb Vasc Biol. 32(9):2271-9 (2012). To analyze whether a certain domain is responsible for the respective phenotype, several deletion mutants were constructed. The deletions were performed in concert with the mapped intracellular domains, deleting TNFR2 domains from the C-terminus. In detail, domain V was deleted in construct TNFR2 Δ18; domains V and IV in TNFR2 Δ59; domains V, IV and III in TNFR2 Δ104, and domains V, IV, III and II in TNFR2 Δ151. The putative functions of the domains are listed in Table 5:

TABLE 5
TNFR2 Domains

| Domain | Putative function |
|---|---|
| I | Unknown |
| II | JNK activation, localization |
| III | JNK activation, localization; TRAF2 degradation |
| IV | TRAF1/2/3 binding |
| V | Bmx, tyrosine kinase/Akt-activation/Contribution |
| | Traf2 binding/NFkb signaling |

To further dissect the influence of the TNFR2 transmembrane domain, a construct harboring a CD8 TM followed by a TNFR2 transmembrane domain was generated, as well as constructs having only the TNFR2 TM followed by CD3z or membrane hybrids, consisting of different amounts of CD8 and TNFR2 derived amino acids.

Fusions of transmembrane domains are composed of a full or part of the human CD8 alpha transmembrane domain fused with a part of the human TNFR2 transmembrane domain. Fusion amino acid sequences used in Example 2 are described in Table 6.

TABLE 6
CD19-CARs transmembrane fusion sequence

| Name of the | | |
|---|---|---|
| Fusion sequence | CD8 sequence | TNFR2 sequence |
| Fusion 1 | IYIWAPLAGTCGVLLLSLVIT | CVIMTQV |
| | (SEQ ID NO: 59) | (SEQ ID NO: 62) |
| Fusion 2 | IYIWAPLAGTCGVLLLSLVIT | VNCVIMTQV |
| | (SEQ ID NO: 60) | (SEQ ID NO: 63) |
| Fusion 3 | IYIWAPLAG | TALGLLIIGVVNCVIMTQV |
| | (SEQ ID NO: 61) | (SEQ ID NO: 64) |

### Results

### CD19-CAR Expression at Cell Surface of Human Tregs

CD19-CARs comprising a TNFR2 transmembrane domain and optionally an entire TNFR2 costimulatory intracellular signaling domain, or fragments thereof, were used to determine if the TNFR2 costimulatory intracellular signaling domain is involved in reducing CAR expression at the cell surface. CD19-CARs comprising a CD8 transmembrane domain and 4-1BB intracellular domain were used as a control.

CD19-CARs comprising a TNFR2 costimulatory intracellular signaling domain and optionally a TNFR2 transmembrane domain or a CD8/TNFR2 fused transmembrane domain were used to determine if the TFNR2 transmembrane domain is involved in reducing CAR expression at the cell surface. CD19-CARs comprising a TNFR2 costimulatory intracellular signaling domain and CD8 transmembrane domain were used as control.

As shown in Table 7, a reduction of CAR expression at the cell surface was observed with CARS comprising a TNFR2 transmembrane domain as well as with CARs comprising a TNFR2 intracellular domain, while not with CARs comprising a CD8 transmembrane domain and 4-1BB intracellular domain in the absence of any TNFR2 domains.

TABLE 7
CD19-CARs expression at cell surface
of human Tregs without activation

| | | Surface CAR | |
|---|---|---|---|
| | Transduced cells | expression % | |
| CAR constructs | (% GFP⁺) | (MFI) | |
| CD19-CAR (CD8TM/4-1BB) | 52% | 76% | (33) |
| CD19-CAR (TNFR2) | 59% | 22% | (2) |
| CD19-CAR (TNFR2 Δ18) | 73% | 30% | (3) |
| CD19-CAR (TNFR2 Δ59) | 73% | 25% | (2) |
| CD19-CAR (TNFR2 Δ104) | 78% | 23% | (3) |
| CD19-CAR (TNFR2 Δ151) | 82% | 52% | (12) |
| CD19-CAR (without) | 84% | 70% | (17) |
| CD19-CAR (CD8TM/TNFR2) | 74% | 80% | (25) |
| CD19-CAR (Fusion 1 + TNFR2) | 73% | 78% | (19) |
| CD19-CAR (Fusion 2 + TNFR2) | 64% | 28% | (2) |
| CD19-CAR (Fusion 3 + TNFR2) | 70% | 27% | (2) |

Globally, these results demonstrate that the TNFR2 transmembrane and costimulatory intracellular signaling domains are both involved in reduced CAR expression at the cell surface of human Tregs.

### CD19-CAR-Specific Activation in Human Tregs

CD19-CARs comprising a TNFR2 transmembrane domain and optionally an entire TNFR2 costimulatory intracellular signaling domain or fragments thereof were used to determine if the TNFR2 costimulatory intracellular signaling domain is involved in CAR activation. CD19-CARs comprising a 4-1BB costimulatory intracellular signaling domain and CD8 transmembrane domain were used as control.

CD19-CARs comprising a TNFR2 costimulatory intracellular signaling domain and optionally a TNFR2 transmembrane domain or a CD8/TNFR2 fused transmembrane domain were used to determine if the TFNR2 transmembrane domain is involved in CAR activation. CD19-CARs comprising a 4-1BB costimulatory intracellular signaling domain and CD8 transmembrane domain were used as control.

As shown in Table 8, a reduction of tonic signaling, evaluated by the expression level of the CD69 early activation marker, was observed with CARs comprising a TNFR2 costimulatory intracellular signaling domain and CD8/TNFR2 fusion transmembrane domains, but not with CARs comprising a CD8 transmembrane domain/4-1BB costimulatory intracellular signaling domain. Despite a low level of expression, TNFR2-derived CARs were efficiently activated with CD19⁺ Daudi cells. In untransduced cells, the CD69 expression background is 9% in the absence of or 32% in the presence of Daudi cells.

TABLE 8
CD19-CARs activation in human Tregs

| | % CD69⁺ pre/post |
|---|---|
| CAR constructs | CAR-ligand binding |
| CD19-CAR (CD8TM/4-1BB) | 20%/79% |
| CD19-CAR (TNFR2) | 12%/59% |
| CD19-CAR (TNFR2 Δ18) | 11%/59% |
| CD19-CAR (TNFR2 Δ59) | 16%/65% |
| CD19-CAR (TNFR2 Δ104) | 19%/81% |
| CD19-CAR (TNFR2 Δ151) | 23%/74% |
| CD19-CAR (without) | 20%/75% |
| CD19-CAR (CD8TM/TNFR2) | 13%/70% |
| CD19-CAR (Fusion 1 + TNFR2) | 14%/70% |
| CD19-CAR (Fusion 2 + TNFR2) | 8%/67% |
| CD19-CAR (Fusion 3 + TNFR2) | 11%/61% |

### CD19-CAR Expression at Cell Surface of Jurkat-T Cells

As with human Treg cells, CD19-CAR expression was also analyzed in Jurkat-Lucia-NFAT cells using the CAR constructs described in Table 5. This reporter cell line is derived from the immortalized human T lymphocyte Jurkat cell.

As shown in Table 9, a reduction of CAR expression at the cell surface was also observed in Jurkat-T cells with CARs comprising a TNFR2 transmembrane domain as well as with CARs comprising a TNFR2 costimulatory intracellular signaling domain, while not with CARs comprising a CD8 transmembrane domain/4-1BB costimulatory intracellular signaling domain construct.

TABLE 9
CD19-CARs expression at cell surface
of Jurkat-T cells without activation

| | | CAR labelling | |
|---|---|---|---|
| | Transduced cells | among GFP⁺ | |
| CAR constructs | (% GFP ) | cells % (MFI) | |
| CD19-CAR (CD8TM/4-1BB) | 88% | 81% | (104) |
| CD19-CAR (TNFR2) | 89% | 31% | (7) |
| CD19-CAR (TNFR2 Δ19) | 83% | 20% | (6) |
| CD19-CAR (TNFR2 Δ59) | 90% | 34% | (5) |
| CD19-CAR (TNFR2 Δ104) | 95% | 29% | (10) |
| CD19-CAR (TNFR2 Δ151) | 94% | 52% | (37) |
| CD19-CAR (without) | 95% | 77% | (53) |
| CD19-CAR (CD8TM/TNFR2) | 80% | 54% | (51) |
| CD19-CAR (Fusion 1 + TNFR2) | 77% | 45% | (34) |
| CD19-CAR (Fusion 2 + TNFR2) | 75% | 19% | (6) |
| CD19-CAR (Fusion 3 + TNFR2) | 72% | 16% | (5) |

These results demonstrate that the TNFR2 transmembrane and the TNFR2 costimulatory intracellular signaling domain are both involved in reduced CAR expression at the cell surface of Jurkat-Tcells.

### TNFR-c-Terminal Deletion Constructs Exhibit Different Surface Expression Pattern and are Functional in CD3z Signaling in Jurkat-NFAT Cells

To test the integrity of the created CD19-CAR constructs, surface expression levels and NFAT signaling were assayed in a Jurkat NFAT-Lucia T cell line.

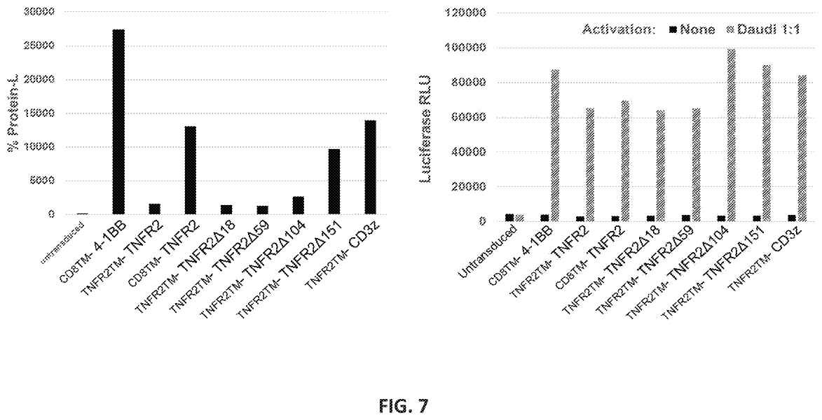

A strong reduction of cell surface expression was observed with the full TNFR2 CAR in comparison to a CD8TM-41BB CAR (FIG. 7). This reduction in expression levels was partially revoked when the TNFR2-TM domain was replaced with the CD8 TM domain. Further, the deletion of TNFR2 domains V, IV and III did not change the cell surface expression detectably from the full TNFR2 domain. However, after deletion of domains II and I, the expression levels increased to half of the levels observed with the 4-1BB construct. At the same time, all constructs continued to exhibit target dependent NFAT signaling. These results together indicate that TNFR2 domains I and II contribute to the reduction of cell surface expression in TNFR2-derived CARs, and that the diminished effect of the deletion mutant is not due to a non-folded protein as the CAR can still exhibit CD3z dependent NFAT-signaling.

### CD19-CAR-Specific Activation in Jurkat-T Cells

CD19-CAR specific activation was also analyzed in Jurkat-Lucia-NFAT cells by measuring NFAT activation after CAR-ligand engagement following incubation with CD19⁺ Daudi cells.

As shown in Table 10, despite a low level of expression, all the TNFR2-derived CARs are efficiently activated with CD19⁺ Daudi cells.

TABLE 10
CD19-CARs activation Jurkat-Lucia-NFAT cells

| | NFAT activation post |
|---|---|
| | CAR-ligand binding |
| CAR constructs | (normalized light units) |
| CD19-CAR (CD8TM/4-1BB) | 100 |
| CD19-CAR (TNFR2) | 73 |
| CD19-CAR (TNFR2 Δ19) | 72 |
| CD19-CAR (TNFR2 Δ59) | 73 |
| CD19-CAR (TNFR2 Δ104) | 114 |
| CD19-CAR (TNFR2 Δ151) | 103 |
| CD19-CAR (without) | 96 |
| CD19-CAR (CD8TM/TNFR2) | 101 |
| CD19-CAR (Fusion 1 + TNFR2) | 74 |
| CD19-CAR (Fusion 2 + TNFR2) | 51 |
| CD19-CAR (Fusion 3 + TNFR2) | 64 |

### Example 3: Evaluation of Anti-CD20 CARs Harboring Different Transmembrane and Intracellular Signaling Domains Derived from Diverse TNFR Receptors (4-1BB, TNFR1 and TNFR2)

### Materials and Methods

The Materials and Methods used were the same as those described in Example 1.

### Results

### Level of Expression of Anti-CD20 CARs Harboring Different TNFR Intracellular Domains

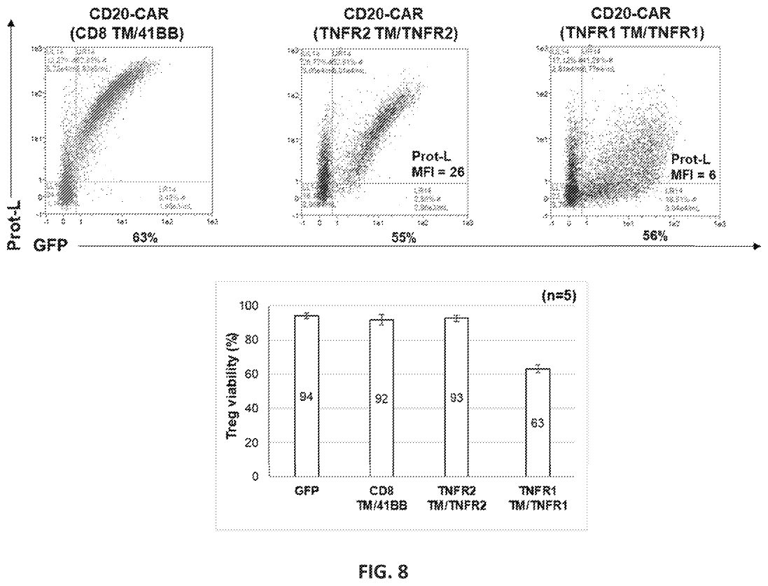

Previously, and in the present study, a decrease in anti-CD20 CAR expression was observed for the construct harboring a TNFR2 transmembrane and intracellular domain, compared to the construct harboring a 4-1 BB intracellular domain (FIG. 8). In this study, new CARs harboring TNFR derived signaling domains were designed using the transmembrane and intracellular domain of human TNFR1 (the nucleotide and amino acid sequences of the CD20-CAR (TNFR1TM-TNFR1-CD3z)-P2A-GFP are SEQ ID NOs: 108 and 109, respectively). As shown in FIG. 8, these new CARs are also expressed at a low level on the cell surface compared to the 4-1BB-derived CAR. However, the CAR comprising the TNFR1-derived sequence is toxic and strongly impacts the survival of FoxP3 Tregs (63% survival). Furthermore, the living/apoptotic cells express the TNFR1-derived CAR with a diffuse pattern (FIG. 8, lower left panel).

### Ligand-Independent Tonic Signaling and Activation Capacity of Anti-CD20 CARs Harboring Different TNFR Intracellular Domains

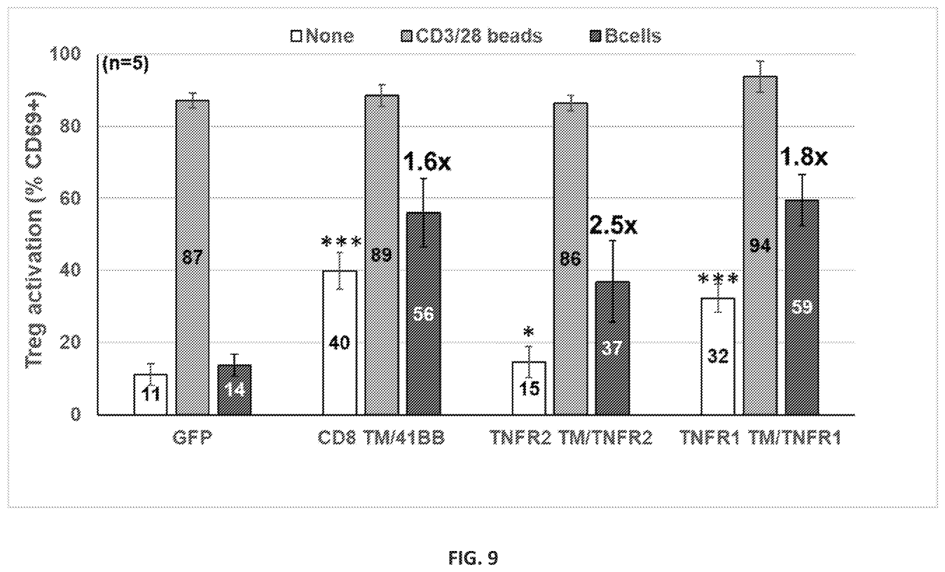

By monitoring CD69 marker expression on unactivated CAR-Treg cells, strong ligand-independent tonic signaling was observed previously with the anti-CD20 CAR construct harboring a 4-1BB intracellular domain. By contrast, this tonic signaling was strongly reduced for the anti-CD20 CAR harboring a TNFR2 transmembrane and intracellular domain. The present study shows that among all of the TNFR-derived CARs (FIG. 9), only the TNFR2-derived CAR was capable of strongly reducing tonic signaling (15% versus the 11% of control), while constructs harboring 4-1BB and TNFR1 sequences harbor tonic signaling of around 40% and 32%, respectively. This result highlights the capacity of TNFR2-derived CARs to specifically decrease ligand independent tonic signaling.

To assess CAR activation, CAR-Treg cells were incubated for 24 hours with autologous B cells expressing the CD20 CAR ligand. As shown in FIG. 9, the most potent CAR activation is observed for the TNFR2-derived CARs (2.5 fold) while all of the other CAR-Tregs are poorly activated (below 1.8 fold).

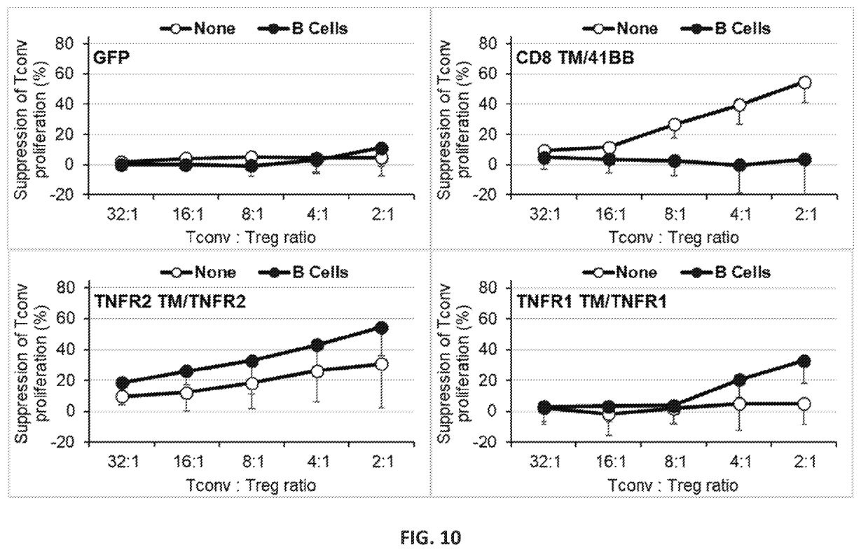

The suppressive activity of CAR-Treg cells was evaluated by monitoring the proliferation of Tconv cells co-cultured with CAR-Tregs in the absence or presence of B cells (B cells) (FIG. 10). Again, the spontaneous suppressive activity of the classical anti-CD20 CAR construct (CD8 TM/4-1BB) was too strong to highlight a CAR-mediated suppressive activity. By contrast, the anti-CD20 CAR derived from TNFR2 allowed the observation of a CAR-mediated suppressive activity. The anti-CD20 CAR derived from TNFR1 exhibited very weak suppressive activity.

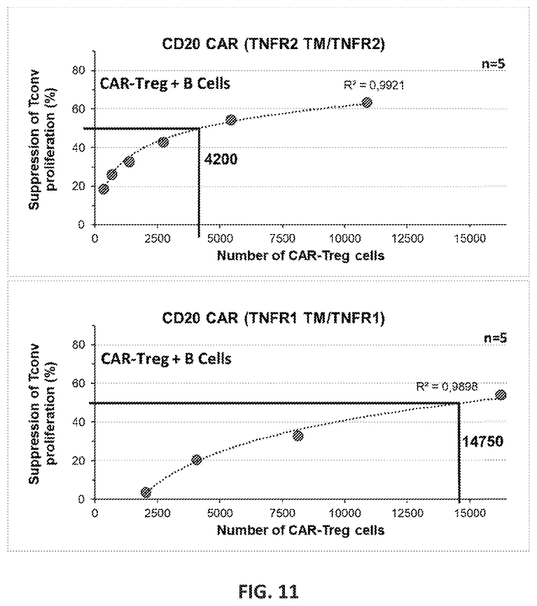

To better define and compare the CAR potency of the various TNFR-derived CARs, the capacity to suppress Tconv proliferation was represented as a function of the absolute number of CAR-Treg cells present in the coculture. As shown in FIG. 11, the TNFR1-derived CAR was highly inefficient (14750 CAR-Tregs to trigger 50% of suppression).

These results demonstrate that among the different human TNFR candidates, the use of TNFR2 transmembrane and TNFR2 costimulatory intracellular signaling domains in CARs is the optimal combination for the design of CAR-Tregs with very low ligand-independent tonic signaling and optimal CAR-dependent Treg activation and suppressive activity.

### Example 4: Treg Markers/Phenotype

### Materials and Methods

The Materials and Methods used were the same as those described in Example 1.

### Results

### Anti-IL-23R CAR-Treg Phenotype in Culture

Results for the IL-23R CAR-Treg phenotype at day 9 are presented in the following table (Table 11). Results are expressed as percentage of cells positive for each marker.

TABLE 11
IL-23R CAR-Treg phenotype at Day 9

| Tregs | transduction | CD4 | CD25 | FoxP3 | Helios | CD62L | CD127 |
|---|---|---|---|---|---|---|---|
| 1132 | GFP | 85.8 | 96.2 | 93.9 | 72.2 | 89.5 | 3.7 |
| | CAR (4- | 47.62 | 95.5 | 81.9 | 18.2 | 75.9 | 23.9 |
| | 1BB/CD3z) | | | | | | |
| 1133 | GFP | 65.6 | 95.0 | 87.9 | 68.3 | 89.5 | 3.6 |
| | CAR (4- | 57.7 | 97.8 | 89.2 | 59.8 | 71.2 | 15.2 |
| | 1BB/CD3z) | | | | | | |
| 1137 | GFP | 94.48 | 97.8 | 94.8 | 55.1 | 93.9 | 3.1 |
| | CAR | 94.25 | 98.5 | 96.3 | 66.6 | 93.3 | 3.3 |
| | (TNFR2/ | | | | | | |
| | CD3z) | | | | | | |
| 1139 | GFP | 90.53 | 97.6 | 94.9 | 51.4 | 95.4 | 2.1 |
| | CAR | 92.73 | 97.8 | 95.3 | 54.5 | 94.1 | 2.8 |
| | (TNFR2/ | | | | | | |
| | CD3z) | | | | | | |
| 1142 | GFP | 93.9 | 94.8 | 85.8 | 30.6 | 82.6 | 11 |
| | CAR (4- | 88.7 | 96.0 | 64.8 | 34.6 | 76.4 | 22 |
| | 1BB/CD3z) | | | | | | |
| | CAR | 93.7 | 65.6 | 74.9 | 38.0 | 79.5 | 14.5 |
| | (TNFR2/ | | | | | | |
| | CD3z) | | | | | | |
| 1144 | GFP | 93.2 | 96.4 | 78.0 | 31.8 | 96.6 | 10.8 |
| | CAR (4- | 86.7 | 95.9 | 50.8 | 23.7 | 70.7 | 25.9 |
| | 1BB/CD3z) | | | | | | |
| | CAR | 92.7 | 96.0 | 62.5 | 31.1 | 89.0 | 10.7 |
| | (TNFR2/ | | | | | | |
| | CD3z) | | | | | | |
| 1148 | GFP | 89.4 | 95 | 95.3 | 63.4 | ND | 3.7 |
| | CAR (4- | 81.1 | 94.1 | 95.1 | 60.4 | | 5.1 |
| | 1BB/CD3z) | | | | | | |
| | CAR | 90 | 94.7 | 95.0 | 64.6 | | 4.2 |
| | (TNFR2/ | | | | | | |
| | CD3z) | | | | | | |
| 1149 | GFP | 89.5 | 95.2 | 95.1 | 66.3 | | 3.4 |
| | CAR (4- | 73.3 | 93.3 | 92.8 | 66.0 | | 9.8 |
| | 1BB/CD3z) | | | | | | |
| | CAR | 91.2 | 95.5 | 92.5 | 61.2 | | 5.7 |
| | (TNFR2/ | | | | | | |
| | CD3z) | | | | | | |

Interestingly, we observed that Tregs transduced with IL-23R-CAR (4-1BB/CD3z), which is highly expressed at the cell surface, demonstrated a loss of stability highlighted by decreases in FoxP3, Helios and CD62L (bold) and an increase in CD127 (bold). However, Tregs transduced with IL-23R-CAR (TNFR2/CD3z), which shows a 9-fold decrease in cell surface expression (191.1±53.2 for the 4-1BB-derived CAR versus 20.5±3.4 for the TNFR2-derived CAR; see Table 3), highlight a robust Treg phenotype after 9 days in culture.

### Anti-CD20 CAR-Treg Phenotype in Culture

Results for the anti-CD20 CAR-Treg phenotype at day 15 are presented in the following table (Table 12). Results are expressed as percentage or mean fluorescence of positive cells for each marker.

TABLE 12
anti-CD20 CAR-Treg phenotype

| | | CD127% (in | FoxP3 MFI | | % CTLA-4+ |
|---|---|---|---|---|---|
| Day 15 | CD25% (in | CD4+ | (in CD4+ | % Helios+ | (in CD4+ |
| (n = 3) | CD4+ pop) | CD25+ pop) | pop) | (in FoxP3+ pop) | CD25+ pop) |
| UT | 79.5 ± 6.4 | 0.8 ± 0.1 | 11.5 ± 1.5 | 62.1 ± 4.4 | 75.4 ± 7.3 |
| GFP | 80.6 ± 3.8 | 0.9 ± 0.1 | 11.1 ± 1.3 | 61.3 ± 2.7 | 74.7 ± 9.1 |
| 4-IBB | 69.9 ± 4.9 | 0.9 ± 0.1 | 10.9 ± 1.0 | 67.2 ± 1.2 | 60.4 ± 5.3 |
| TNFR2 | 72.9 ± 2.5 | 0.8 ± 0.0 | 11.3 ± 1.3 | 72.9 ± 2.4 | 63.0 ± 5.4 |

Interestingly, the Treg phenotype is not statistically different between the different CAR-Treg constructs and the control transduced with GFP only.

### Example 5: In Vivo Use of CAR-Tregs

### Materials and Methods

### GvHD

One-day prior to human HLA-A2⁺ PBMC injection, a total of 23 NSG mice (8 weeks) were conditioned with IP injection of Busulfan 30 mg/kg to favor human cell engraftment. One day later (at Day 0), the mice were injected IV with HLA-A*02⁺ human PBMCs (5×10⁶ PBMCs per mouse) and human HLA-A2 CAR Tregs were injected in a 1:1 PBMC:CAR-Treg ratio. Three times a week, the mice were weighed (BW) and GvHD score was assessed. Blood samples were taken every week. At the time of sacrifice, spleen and lung were also analyzed. Human Tregs were isolated from HLA-A2 negative healthy volunteers and transduced as previously described.

### Results

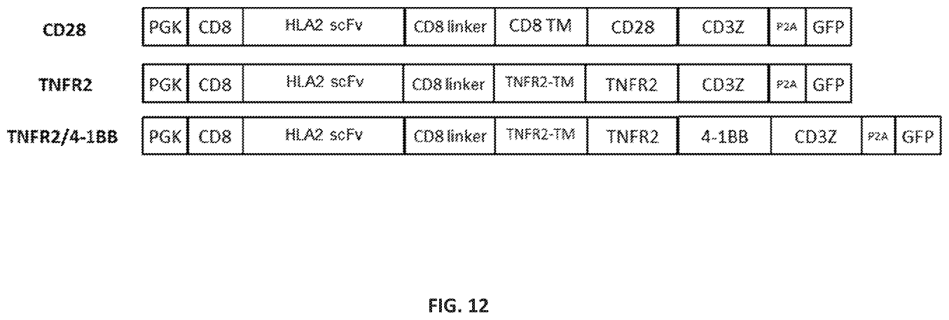

CAR Tregs comprising CD28, TNFR2 or TNFR2+4-1BB cosignaling domains were tested for their activity in vivo in a GvHD mouse model. Three different CAR constructs (FIG. 12) were transduced in CD4⁺CD25⁺CD127^{low}, CD45RA⁺ human Tregs as described previously.

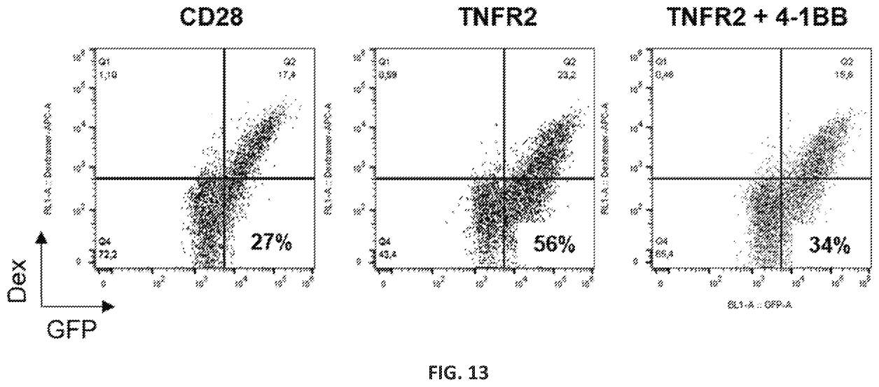

Transduction efficiency was assessed by the percentage of GFP positive cells expression and CAR expression was monitored using Dextramer®. As shown in FIG. 13, Tregs were transduced at 27%, 56% and 34% with CD28 or TNFR2 or TNFR2+4-1BB HLA*A2 CARs, respectively.

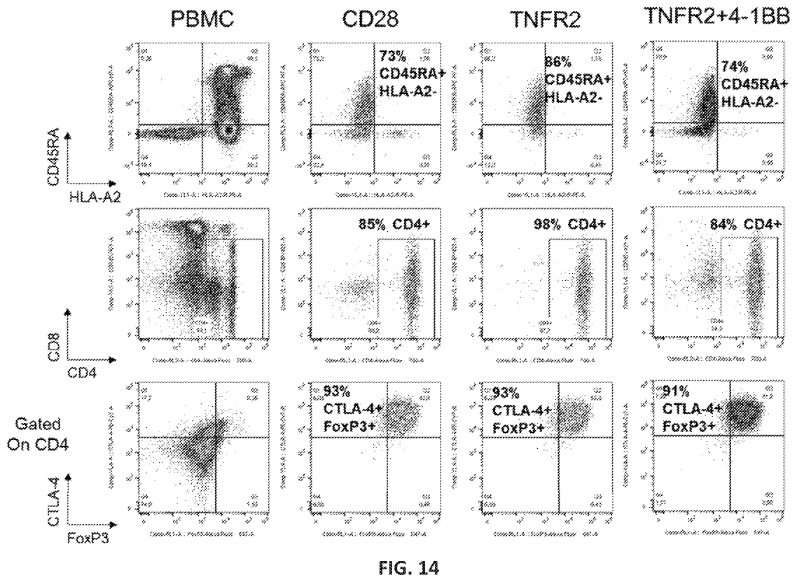

The three different HLA*A2 CAR-Tregs displayed a robust Treg phenotype of CD4⁺ CD45RA⁺ FoxP3⁺ CTLA-4⁺ (FIG. 14).

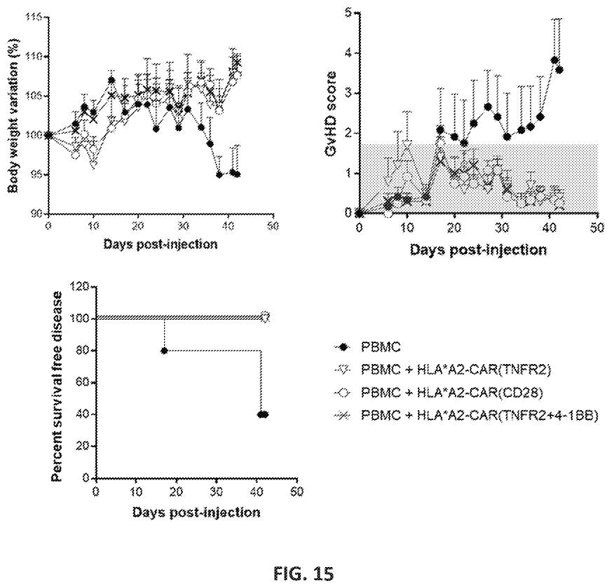

HLA*A2 CAR-Tregs were injected into NSG mice at a 1:1 ratio (PBMC:CAR-Tregs). HLA*A2 CAR-Tregs comprising TNFR2, TNFR2+4-1BB or CD28 cosignaling domains were all able to control GvHD (FIG. 15).

TABLE 13
Sequences

| SID¹ | Type² | Description |
|---|---|---|
| 1-2 | aa | anti-CD19 scFvs |
| 3-6 | aa | anti-CD20 scFvs |
| 7 | aa | anti-desmo 3 (domains 1-4) |
| 8-11 | aa | linkers |
| 12 | nt | linker |
| 13 | aa | KIR₂DS₂ hinge domain |
| 14 | aa | CD8 hinge domain |
| 15 | nt | CD8 hinge domain |
| 16 | aa | IgG4 hinge domain |
| 17 | nt | IgG4 hinge domain |
| 18 | aa | IgD hinge domain |
| 19 | nt | IgD hinge domain |
| 20 | aa | CD28 hinge domain |
| 21 | nt | CD28 hinge domain |
| 22 | aa | TNFR2 transmembrane domain |
| 23 | nt | TNFR2 transmembrane domain |
| 24 | aa | CD8 transmembrane domain |
| 25 | nt | CD8 transmembrane domain |
| 26 | aa | CD28 transmembrane domain |
| 27 | nt | CD28 transmembrane domain |
| 28-31 | aa | CD3 zeta intracellular signaling domains |
| 32-33 | nt | CD3 zeta intracellular signaling domains |
| 34 | aa | TNFR2 intracellular domain |
| 35 | nt | TNFR2 intracellular domain |
| 36 | aa | 4-1BB intracellular domain |
| 37 | nt | 4-1BB intracellular domain |
| 38 | aa | CD27 intracellular domain |
| 39 | nt | CD27 intracellular domain |
| 40 | aa | CD28 intracellular domain |
| 41 | nt | CD28 intracellular domain |
| 42 | aa | CD8 leader sequence |
| 43-44 | aa | TAGs |
| 45 | aa | P2A TAG |
| 46 | aa | GFP TAG |
| 47 | aa | streptavidin tag |
| 48 | aa | CD8 H-TNFR2 TM-TNFR2-3z |
| 49 | aa | CD8 H-TNFR2 TM-TNFR2Δ18-3z |
| 50 | aa | CD8 H-TNFR2 TM-TNFR2Δ59-3z |
| 51 | aa | CD8 H-TNFR2 TM-TNFR2Δ104-3z |
| 52 | aa | CD8 H-TNFR2 TM-3z |
| 53 | aa | CD8 H-3z-TNFR2 TM |
| 54 | aa | CD8 H-CD8 TM- TNFR2-3z |
| 55 | aa | CD8 H-FUSED 1 TM- TNFR2-3z |
| 56 | aa | CD8 H-FUSED 2 TM- TNFR2-3z |
| 57 | aa | CD8 H-FUSED 3 TM- TNFR2-3z |
| 58 | aa | LAGLIDADG motif |
| 59 | aa | Fusion 1 - CD8 SEQ |
| 60 | aa | Fusion 2 - CD8 SEQ |
| 61 | aa | Fusion 3 - CD8 SEQ |
| 62 | aa | Fusion 1 - TNFR2 SEQ |
| 63 | aa | Fusion 2 - TNFR2 SEQ |
| 64 | aa | Fusion 3 - TNFR2 SEQ |
| 65-67 | aa | anti-IL-23R scFvs |
| 68-107 | aa | anti-HLA-A2 scFvs |
| 108 | nt | CD20-CAR (TNFR1TM-TNFR1-CD3z)-P2A-GFP |
| 109 | aa | CD20-CAR (TNFR1TM-TNFR1-CD3z)-P2A-GFP |
| 110 | aa | CD8 H-TNFR2 TM-TNFR2Δ151-3z |
| 111-117 | aa | linkers |

¹SID: SEQ ID NO:
²aa: amino acid, nt: nucleotide

1. A chimeric antigen receptor (CAR) comprising an extracellular binding domain, a transmembrane domain, and an intracellular domain, wherein
the transmembrane domain comprises a human tumor necrosis factor receptor 2 (TNFR2) transmembrane domain or a fragment or variant thereof, or
(ii) the intracellular domain comprises a human TNFR2 costimulatory intracellular signaling domain or a fragment or variant thereof, or
(iii) both (i) and (ii).

2. The CAR according to claim 1, further comprising an extracellular hinge domain.

3. The CAR according to claim 2, wherein the hinge domain comprises a hinge region of human CD8 or CD28.

4. The CAR according to claim 3, wherein the hinge domain comprises the sequence of SEQ ID NO: 14 or a sequence having at least about 70% identity with SEQ ID NO: 14.

5. The CAR according to any one of the preceding claims, wherein the intracellular domain comprises an immune cell primary intracellular signaling domain.

6. The CAR according to claim 5, wherein the intracellular domain comprises a T cell primary intracellular signaling domain of human CD3.

7. The CAR according to any one of the preceding claims, wherein the intracellular domain comprises a primary intracellular signaling domain of human CD3 zeta, optionally comprising the sequence of SEQ ID NO: 28, 29, 30 or 31 or a sequence having at least about 70% identity with SEQ ID NO: 28, 29, 30 or 31.

8. The CAR according to any one of the preceding claims, wherein the CAR comprises:
an extracellular binding domain,
an extracellular hinge domain comprising a hinge region of human CD8 or CD28,
a transmembrane domain comprising a human TNFR2 transmembrane domain or a fragment or variant thereof, and
an intracellular domain comprising a primary intracellular signaling domain of human CD3 zeta.

9. The CAR according to any one of the preceding claims, wherein the CAR comprises:
an extracellular binding domain,
an extracellular hinge domain comprising a hinge region of human CD8 or CD28,
a transmembrane domain, and
an intracellular domain comprising a human TNFR2 costimulatory intracellular signaling domain or a fragment or variant thereof, and a primary intracellular signaling domain of human CD3 zeta.

10. The CAR according to any one of the preceding claims, wherein the CAR comprises:
an extracellular binding domain,
an extracellular hinge domain comprising a hinge region of human CD8 or CD28,
a transmembrane domain comprising a human TNFR2 transmembrane domain or a fragment or variant thereof, and
an intracellular domain comprising a human TNFR2 costimulatory intracellular signaling domain or a fragment or variant thereof, and a primary intracellular signaling domain of human CD3 zeta.

11. The CAR according to any one of the preceding claims, wherein the transmembrane domain comprises at least eight contiguous amino acids of SEQ ID NO: 22 or of a sequence having at least about 70% identity with SEQ ID NO: 22.

12. The CAR according to claim 11, wherein the transmembrane domain comprises at least eight contiguous amino acid residues of SEQ ID NO: 22 in combination with amino acid residues from a transmembrane domain of a protein that is not TNFR2.

13. The CAR according to any one of the preceding claims, wherein the transmembrane domain comprises the amino acid sequence of VNCVIMTQV (SEQ ID NO: 63).

14. The CAR according to any one of the preceding claims, wherein the intracellular domain comprises at least 30 contiguous amino acid residues of SEQ ID NO: 34 or of a sequence having at least about 70% identity with SEQ ID NO: 34.

15. The CAR according to claim 14, wherein said intracellular domain comprises at least 30 contiguous amino acid residues of SEQ ID NO: 34 in combination with amino acid residues from a costimulatory intracellular signaling domain of a protein that is not TNFR2.

16. The CAR according to any one of the preceding claims, wherein said intracellular signaling domain comprises residues 1-70, 1-115, or 1-156 of SEQ ID NO: 34.

17. The CAR according to any one of the preceding claims, comprising:
an extracellular binding domain,
an extracellular hinge domain comprising a CD8 hinge region of SEQ ID NO: 14,
a transmembrane domain comprising a TNFR2 transmembrane domain of SEQ ID NO: 22, and
an intracellular domain comprising:
a) a primary human CD3 zeta intracellular signaling domain of SEQ ID NO: 28, 29, 30, or 31, and
b) a TNFR2 costimulatory intracellular signaling domain of SEQ ID NO: 34.

18. The CAR according to any one of the preceding claims, wherein the extracellular binding domain is an antibody or an antigen-binding fragment thereof.

19. The CAR according to claim 18, wherein the extracellular binding domain is a single chain variable fragment (scFv).

20. The CAR according to any one of the preceding claims, wherein the extracellular binding domain specifically binds
(a) an autoantigen, wherein the autoantigen is optionally IL-23 receptor (IL-23R);
(b) a B cell antigen, optionally selected from CD19 and CD20; or
(c) an allogeneic HLA class I or class II molecule, wherein the class I molecule is optionally HLA-A2.

21. A nucleic acid sequence encoding the CAR according to any one of claims 1-20.

22. A vector comprising the nucleic acid sequence according to claim 21.

23. A host cell comprising the nucleic acid sequence of claim 21 or the vector of claim 22.

24. A population of immune cells expressing the CAR according to any one of claims 1-20.

25. The immune cell population according to claim 24, wherein the immune cells are selected from the group consisting of T cells, natural killer (NK) cells, αβ T cells, γδ T cells, double negative (DN) cells, regulatory immune cells, regulatory T (Treg) cells, effector immune cells, effector T cells, B cells, and myeloid-derived cells, and any combination thereof, wherein the immune cells are optionally human cells.

26. The immune cell population according to claim 24, wherein the population comprises Treg cells, wherein the Treg cells are optionally human cells.

27. The immune cell population according to claim 26, wherein the population comprises human Treg cells expressing a CAR comprising:
an extracellular binding domain,
a hinge domain comprising a hinge region of human CD8,
a human TNFR2 transmembrane domain, and
an intracellular domain comprising a human TNFR2 costimulatory intracellular signaling domain and a primary intracellular signaling domain of human CD3 zeta.

28. A pharmaceutical composition comprising an immune cell expressing the CAR according to any one of claims 1-20, or a host cell according to claim 23, or an immune cell population according to any one of claims 24-27, and a pharmaceutically acceptable excipient.

29. A method for treating a disease or disorder in a human subject in need thereof, comprising administering to the subject the pharmaceutical composition according to claim 28.

30. A chimeric antigen receptor of any one of claims 1-20, or an immune cell population according to any one of claims 24-27, for use in the treatment of a disease or disorder in a human subject in need thereof.

31. Use of a chimeric antigen receptor according to any one of claims 1-20, or an immune cell population according to any one of claims 24-27, for the manufacture of a medicament for the treatment of a disease or disorder in a human subject in need thereof.

32. The method according to claim 29, the CAR or immune cell population for use according to claim 30, or the use according to claim 31, wherein the disease or disorder is selected from the group consisting of an inflammatory disease, an autoimmune disease, an allergic disease, an organ transplantation condition, a cancer, and an infectious disease.

33. The method according to claim 29, the CAR or immune cell population for use according to claim 30, or the use according to claim 31, wherein the human subject is in need of immunosuppression and the CAR is expressed in Treg cells in the human subject.

34. The method according to claim 33, the CAR or immune cell population for use according to claim 33, or the use according to claim 33, wherein the disease or disorder is an inflammatory disease, an autoimmune disease, an allergic disease, or an organ transplantation condition.

35. The method according to claim 34, the CAR or immune cell population for use according to claim 34, or the use according to claim 34, wherein the organ transplantation condition is graft rejection or graft-versus-host disease.

36. A chimeric antigen receptor of any one of claims 1-20, or an immune cell population according to any one of claims 24-27, for use as a medicament.
